(19) United States
(12) Patent Application Publication
(10) Pub. No.: US 2024/0208909 A1
(43) Pub. Date: Jun. 27, 2024

(54) SUBSTITUTED QUINOLINE DERIVATIVES HAVING SOS1 INHIBITION ACTIVITIES AND USES THEREOF

(71) Applicant: Accutar Biotechnology Inc., Cranbury, NJ (US)

(72) Inventors: Robert Zhiyong Luo, New City, NY (US); Jie Su, New York, NY (US); Pin Huang, Shanghai (CN); Ke LIU, Shanghai (CN); Wei HE, Zionsville, IN (US); Jie FAN, New York, NY (US); Yimin Qian, Plainsboro, NJ (US)

(73) Assignee: Accutar Biotechnology Inc., Cranbury, NJ (US)

(21) Appl. No.: 18/526,770

(22) Filed: Dec. 1, 2023

Related U.S. Application Data
(60) Provisional application No. 63/385,836, filed on Dec. 2, 2022.

(51) Int. Cl. C07D 215/44; A61P 35/00; C07D 215/54; C07D 401/04; C07D 405/12; C07D 417/12; C07D 471/10; C07D 487/04; C07D 487/10; C07D 491/08

(52) U.S. Cl. CPC C07D 215/44; A61P 35/00; C07D 215/54; C07D 401/04; C07D 405/12; C07D 417/12; C07D 471/10; C07D 487/04; C07D 487/10; C07D 491/08

(57) ABSTRACT

The present disclosure relates to novel SOS1 inhibitors, pharmaceutical compositions containing such compounds, and their use in prevention and treatment of cancer and related diseases and conditions.

This application claims the benefit of priority to U.S. Provisional Patent Application No. 63/385,836, filed Dec. 2, 2022, which is hereby incorporated by reference in its entirety.

### FIELD OF THE DISCLOSURE

The present disclosure relates to SOS1 inhibitors. In particular, the present disclosure relates to SOS1 inhibitors/compounds that block RAS activation by disrupting the RAS-SOS1 interaction, pharmaceutical compositions comprising the compounds and methods of use therefor.

### BACKGROUND OF THE DISCLOSURE

The KRAS, NRAS and HRAS genes encode a set of closely related small GTPase proteins KRas, NRas and HRas, collectively referred to herein as the Ras proteins or Ras, that share 82-90% overall sequence identity. The Ras proteins are critical components of signaling pathways transmitting signals from cell-surface receptors to regulate cellular proliferation, survival and differentiation. Ras functions as a molecular switch cycling between an inactive GDP-bound state and an active GTP-bound state. The GDP/GTP cycle of Ras is tightly regulated in cells by guanine nucleotide exchange factors (GEFs), such as Son of Sevenless (SOS). SOS has two human isoforms, SOS1 and SOS2. SOS1 features two distinct RAS binding sites, namely a catalytic site and an additional allosteric RAS binding site (see SM Margarit, et al., Structural evidence for feedback activation by Ras. GTP of the Ras-specific nucleotide exchange factor SOS. Cell 112, 685-695 (2003)).

Ras mutations are frequently found in cancer and approximately 30% of all human cancers have a mutation in KRAS, NRAS or HRAS genes. Oncogenic Ras is typically, but not exclusively, associated with mutations at glycine 12, glycine 13 or glutamine 61 of Ras. These residues are located at the active site of Ras and mutations impair intrinsic and/or GAP-catalyzed GTPase activity favoring the formation of GTP bound Ras and aberrant activation of down-stream effector pathways. SOS1 is significantly involved in activating mutant Ras and decreasing the amount of SOS1 binding to Ras reduces the chances of survival for tumor cells containing a Ras mutation. Moreover, SOS1 mutations result in Ras over-expression and are further implicated in cancer.

There are several tumor types that exhibit a high frequency of activating mutations in KRAS including pancreatic (˜90% prevalence), colorectal (˜40% prevalence) and non-small cell lung cancer (˜30% prevalence). KRAS mutations are also found in other cancer types including multiple myeloma, uterine cancer, bile duct cancer, stomach cancer, bladder cancer, diffuse large B cell lymphoma, rhabdomyosarcoma, cutaneous squamous cell carcinoma, cervical cancer, testicular germ cell cancer and others.

There remains a need for new drugs that efficiently inhibit the activation of KRAS by SOS1. These types of drugs could provide new medical treatments for lung cancer, pancreatic cancer, or colorectal cancer, especially those who have been diagnosed to have such cancers characterized by a KRAS or SOS1 mutation and including those having cancer that progressed after chemotherapy.

Thus, disclosed herein are novel substituted quinoline-based compounds that exhibit SOS1 inhibition activity. The disclosed compounds are useful for the treatment of cancers, such as breast cancer, lung cancer, pancreatic cancer, small bowel cancer, colorectal cancer, gall bladder cancer, thyroid cancer, liver cancer, bile duct cancer, ovarian cancer, endometrial cancer, cervical cancer, bladder cancer, prostate cancer, esophageal cancer, and blood cancer.

### SUMMARY OF THE DISCLOSURE

The disclosure provides compounds, compositions, and methods for modulating the activity of SOS1.

In one aspect, the disclosure provides compounds which act as SOS1 inhibitors.

Disclosed are compounds of Formula (1) or a tautomer, stereoisomer or a mixture of stereoisomers, or a pharmaceutically acceptable salt, or hydrate, or deuterated derivative thereof:

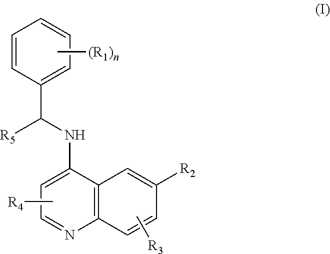

wherein:
R₁ is independently chosen from C₁-C₅ alkyl, halogen, C₁-C₅ alkoxy, C₁-C₄ haloalkyl, and cyano, and n is 0, 1, or 2;
R₂ is chosen from a 4- to 10-membered heteromonocyclic group, fused heterocyclic group, and bridged or spiro heterocyclic group, wherein each of the heteromonocyclic group, fused heterocyclic group, and bridged or spiro heterocyclic group is independently substituted with 0, 1, 2, or 3 R^{a};
R₃ is absent or each R₃ is alkoxyl, C₁-C₅ alkyl, or halo group;
R₄ is absent or each R₄ is cyano or alkyl;
R₅ is a C₁-C₄ alkyl;
R^{a} is absent or each R^{a} is independently chosen from a hydrogen, alkoxyl group, deuterated alkoxyl group, deuterated alkyl group, hydroxyl, halo, haloalkyl, alkyl, and 4- to 5-member heterocycle which is independently substituted with 0 or 1 R₆, C(O)R₇, C(O)NR₇, N(R₇)₂, NR₇C(O)R₇, NR₇C(O)OR₇, or C(O)OR₇ group;
R₆ is absent or alkyl; and
each R₇ is independently chosen from hydrogen, alkoxyl, N(CH₃)₂, NHCH₃, a 3- to 6-member cycloalkyl, a 3- to 6-member heterocycle, C₁-C₅ alkyl, CH₂—CN, and haloalkyl.

In some embodiments, R₂ is chosen from,

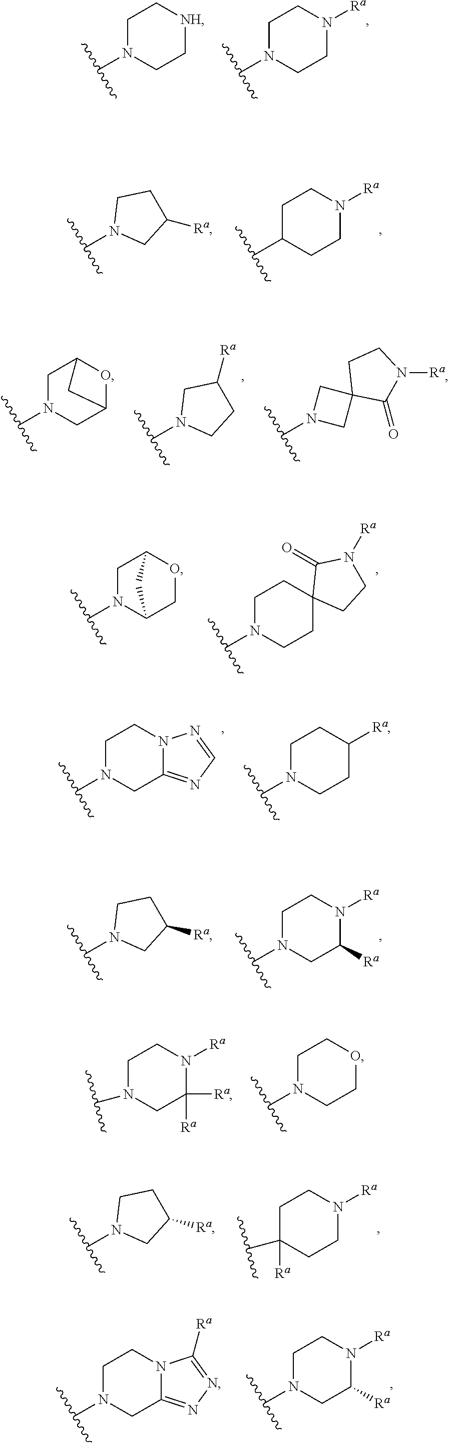

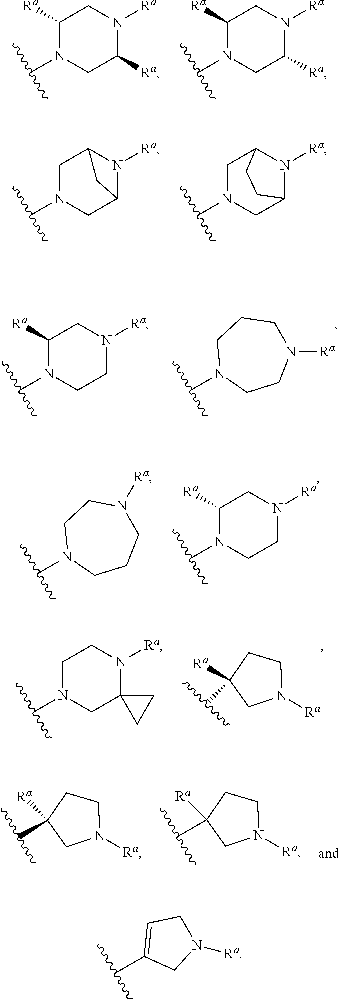

In some embodiments, R^{a} is chosen from

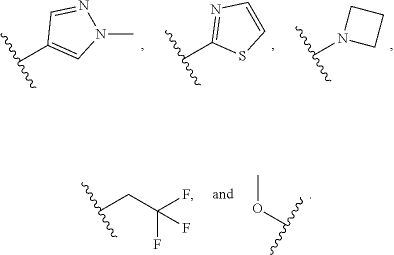

In some embodiments, R₇ is chosen from

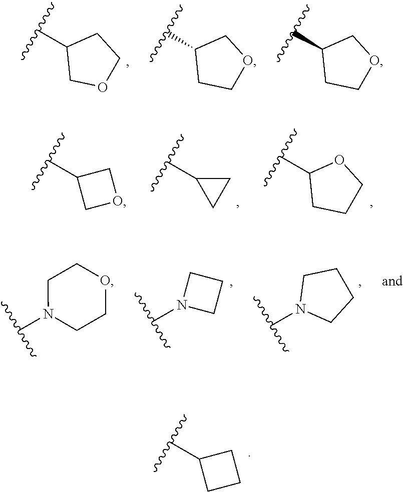

In some embodiments, the compound of Formula (1) may encompass one or more stereoisomers and/or a mixture of stereoisomers. In some embodiments, the compound of Formula (1) may encompass one or more racemic isomers and/or enantiomeric isomers.

Also disclosed herein is a method of treating cancer in a subject in need thereof, comprising administering to said subject a compound of Formula (1) or pharmaceutically acceptable salt thereof, or a pharmaceutical composition comprising a compound of Formula (1) or a pharmaceutically acceptable salt thereof. In at least one embodiment, the pharmaceutical composition of the present disclosure may be for use in (or in the manufacture of medicaments for) the treatment of cancer in the subject in need thereof. In some embodiments, the cancer is chosen from breast cancer, lung cancer, pancreatic cancer, small bowel cancer, colorectal cancer, gall bladder cancer, thyroid cancer, liver cancer, bile duct cancer, ovarian cancer, endometrial cancer, cervical cancer, bladder cancer, prostate cancer, esophageal cancer, and blood cancer.

In some embodiments, a therapeutically effective amount of a pharmaceutical composition of the present disclosure may be administered to a subject diagnosed with cancer. In some embodiments, the amount of a compound of Formula I is in a range of from 1 mg to 1000 mg.

### BRIEF DESCRIPTION OF FIGURES

The accompanying drawings, which are incorporated in and constitute a part of this specification, illustrate the disclosed embodiments and, together with the description, explain the principles of the disclosed embodiments.

FIG. 1 illustrates tumor volume trace after administering Compound 76 and vehicle to tumor-bearing female NCG mice.

FIG. 2 illustrates the overall structure of the complex of SOS1-Compound 76.

FIG. 3 illustrates the absolute stereochemistry of Compound 76 determined by X-ray density.

FIG. 4 illustrates the interactions between Compound 76 and SOS1.

### DETAILED DESCRIPTION OF THE DISCLOSURE

### Definitions

A dash (“-”) that is not between two letters or symbols is used to indicate a point of attachment for a substituent. For example, —CN is attached through the carbon atom.

When a range of values is listed, it is intended to encompass each value and all sub-ranges within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “acyl” as used herein refers to R—C(O)— groups such as, but not limited to, (alkyl)-C(O)—, (alkenyl)-C(O)—, (alkynyl)-C(O)—, (aryl)-C(O)—, (cycloalkyl)-C(O)—, (heteroaryl)-C(O)—, and (heterocyclyl)-C(O)—, wherein the group is attached to the parent molecular structure through the carbonyl functionality. In some embodiments, it is a C₁₋₁₀ acyl radical which refers to the total number of chain or ring atoms of the, for example, alkyl, alkenyl, alkynyl, aryl, cycloalkyl, or heteroaryl, portion plus the carbonyl carbon of acyl. For example, a C₄-acyl has three other ring or chain atoms plus carbonyl.

The term “alkenyl” as used herein refers to an unsaturated straight or branched hydrocarbon having at least one carbon-carbon double bond, such as a straight or branched group of 2-8 carbon atoms, referred to herein as (C₂-C₈)alkenyl. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, and 4-(2-methyl-3-butene)-pentenyl.

The term “alkyl” as used herein refers to a saturated straight or branched hydrocarbon, such as a straight or branched group of 1-8 carbon atoms, referred to herein as C₁₋₈ alkyl. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl. In some embodiments, “alkyl” is a straight-chain hydrocarbon. In some embodiments, “alkyl” is a branched hydrocarbon.

The term “alkoxy” means a straight or branched chain saturated hydrocarbon containing 1-12 carbon atoms containing a terminal “O” in the chain, e.g., —O(alkyl). Examples of alkoxy groups include, without limitation, methoxy, ethoxy, propoxy, butoxy, t-butoxy, or pentoxy groups.

The term “alkylene” as used herein refers to a divalent alkyl radical. Representative examples of C₁₋₁₀ alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene and n-decylene.

The term “alkynyl” as used herein refers to an unsaturated straight or branched hydrocarbon having at least one carbon-carbon triple bond, such as a straight or branched group of 2-8 carbon atoms, referred to herein as (C₂-C₈)alkynyl. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl.

The term “aryl” as used herein refers to a mono-, bi-, or other multi-carbocyclic, aromatic ring system with 5 to 14 ring atoms. The aryl group can optionally be fused to one or more rings chosen from aryls, cycloalkyls, heteroaryls, and heterocyclyls. The aryl groups of this present disclosure can be substituted with groups chosen from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include but are not limited to a monocyclic aromatic ring system, wherein the ring comprises 6 carbon atoms.

The term “cyano” as used herein refers to —CN.

The term “cycloalkyl” as used herein refers to a saturated or unsaturated cyclic, bicyclic, or bridged bicyclic hydrocarbon group of 3-16 carbons, or 3-8 carbons, referred to herein as “(C₃-C₈)cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl (saturated or partially unsaturated), aryl, or heterocyclyl groups, to form a bicycle, tetracycle, etc. The term “cycloalkyl” also includes bridged and spiro-fused cyclic structures which may or may not contain heteroatoms.

The terms “halo” or “halogen” as used herein refer to —F, —C₁, —Br, and/or —I.

“Haloalkyl” means an alkyl group substituted with one or more halogens. Examples of haloalkyl groups include, but are not limited to, trifluoromethyl, difluoromethyl, pentafluoroethyl, trichloromethyl, etc.

The term “heteroaryl” as used herein refers to a mono-, bi-, or multi-cyclic, aromatic ring system containing one or more heteroatoms, for example 1-3 heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Exemplary heteroaryl groups include, but are not limited to, a monocyclic aromatic ring, wherein the ring comprises 2-5 carbon atoms and 1-3 heteroatoms, referred to herein as “(C₂-C₅)heteroaryl.” Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups also include, but are not limited to, a bicyclic aromatic ring, wherein the ring comprises 5-14 carbon atoms and 1-3 heteroatoms, referred to herein as “(C₅-C₁₄)heteroaryl.” Representative examples of heteroaryl include, but are not limited to, indazolyl, indolyl, azaindolyl, indolinyl, benzotriazolyl, benzoxadiazolyl, imidazolyl, cinnolinyl, imidazopyridyl, pyrazolopyridyl, pyrrolopyridyl, quinolinyl, isoquinolinyl, quinazolinyl, quinazolinonyl, indolinonyl, isoindolinonyl, tetrahydronaphthyl, tetrahydroquinolinyl, tetrahydroisoquinolinyl, and the like.

The terms “heterocycle,” “heterocyclyl,” or “heterocyclic” as used herein each refer to a saturated or unsaturated 3- to 18-membered ring containing one, two, three, or four heteroatoms independently chosen from nitrogen, oxygen, phosphorus, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heterocycles also include bicyclic, tricyclic, and tetracyclic groups in which any of the above heterocyclic rings is fused to one or two rings independently chosen from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

The terms “hydroxy” and “hydroxyl” as used herein refer to —OH.

“Spirocycloalkyl” or “spirocyclyl” means carbogenic bicyclic ring systems with both rings connected through a single atom. The rings can be different in size and nature, or identical in size and nature. Examples include spiropentane, spriohexane, spiroheptane, spirooctane, spirononane, or spirodecane. One or both of the rings in a spirocycle can be fused to another carbocyclic ring, heterocyclic ring, aromatic ring, or heteroaromatic ring. A (C₃₋₁₂)spirocycloalkyl is a spirocycle containing between 3 and 12 carbon atoms.

“Spiroheterocycloalkyl” or “spiroheterocyclyl” means a spirocycle wherein at least one of the rings is a heterocycle one or more of the carbon atoms can be substituted with a heteroatom (e.g., one or more of the carbon atoms can be substituted with a heteroatom in at least one of the rings). One or both of the rings in a spiroheterocycle can be fused to another ring carbocyclic, heterocyclic, aromatic, or heteroaromatic ring.

“Isomers” means compounds having the same number and kind of atoms, and hence the same molecular weight, but differing with respect to the arrangement or configuration of the atoms in space.

“Stereoisomer” or “optical isomer” mean a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds of the disclosure which may give rise to stereoisomerism, the disclosure contemplates stereoisomers and mixtures thereof. The compounds of the disclosure and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

It is well-known in the art that the biological and pharmacological activity of a compound is sensitive to the stereochemistry of the compound. For example, enantiomers often exhibit strikingly different biological activity including differences in pharmacokinetic properties, including metabolism, protein binding, and the like, and pharmacological properties, including the type of activity displayed, the degree of activity, toxicity, and the like. Thus, one skilled in the art will appreciate that one enantiomer may be more active or may exhibit beneficial effects when enriched relative to the other enantiomer or when separated from the other enantiomer. Additionally, one skilled in the art would know how to separate, enrich, or selectively prepare the enantiomers of the compounds of the disclosure from this disclosure and the knowledge of the prior art.

Thus, although the racemic form of drug may be used, it is often less effective than administering an equal amount of enantiomerically pure drug; indeed, in some cases, one enantiomer may be pharmacologically inactive and would merely serve as a simple diluent. For example, although ibuprofen had been previously administered as a racemate, it has been shown that only the S-isomer of ibuprofen is effective as an anti-inflammatory agent (in the case of ibuprofen, however, although the R-isomer is inactive, it is converted in vivo to the S-isomer, thus, the rapidity of action of the racemic form of the drug is less than that of the pure S-isomer). Furthermore, the pharmacological activities of enantiomers may have distinct biological activity. For example, S-penicillamine is a therapeutic agent for chronic arthritis, while R-penicillamine is toxic. Indeed, some purified enantiomers have advantages over the racemates, as it has been reported that purified individual isomers have faster transdermal penetration rates compared to the racemic mixture. See U.S. Pat. Nos. 5,114,946 and 4,818,541.

The compounds of the disclosure may contain one or more chiral centers and/or double bonds and, therefore, exist as stereoisomers, such as geometric isomers, enantiomers, or diastereomers. The term “stereoisomers” when used herein consists of all geometric isomers, enantiomers, or diastereomers. These compounds may be designated by the symbols “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. In some embodiments, an enantiomer or stereoisomer may be provided substantially free of the corresponding enantiomer.

In some embodiments, the compound is a racemic mixture of (S)- and (R)-isomers. In other embodiments, provided herein is a mixture of compounds wherein individual compounds of the mixture exist predominately in an (S)- or (R)-isomeric configuration. For example, the compound mixture has an (S)-enantiomeric excess of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more. In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5%, or more. In some other embodiments, the compound mixture has an (R)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

Individual stereoisomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by: (1) attachment of a mixture of enantiomers to a chiral auxiliary, separation of the resulting mixture of diastereomers by recrystallization or chromatography and liberation of the optically pure product from the auxiliary; (2) salt formation employing an optically active resolving agent; or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Thus, if one enantiomer is pharmacologically more active, less toxic, or has a preferred disposition in the body than the other enantiomer, it would be therapeutically more beneficial to administer that enantiomer preferentially. In this way, the patient undergoing treatment would be exposed to a lower total dose of the drug and to a lower dose of an enantiomer that is possibly toxic or an inhibitor of the other enantiomer.

The compounds disclosed herein may exist as tautomers and both tautomeric forms are intended to be encompassed by the scope of the present disclosure, even if only one tautomeric structure is depicted.

Additionally, unless otherwise stated, structures described herein are also meant to include compounds that differ only in the presence of one or more isotopically enriched atoms. For example, compounds having the present structures except for the replacement of hydrogen by deuterium (²H) or tritium (³H), or the replacement of a carbon by a ¹³C- or ¹⁴C-carbon atom are within the scope of this disclosure. Such compounds may be useful as, for example, analytical tools, probes in biological assays, or therapeutic agents.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutically acceptable composition” as used herein refers to a composition comprising at least one compound as disclosed herein formulated together with one or more pharmaceutically acceptable carriers.

The term “pharmaceutically acceptable prodrugs” as used herein represents those prodrugs of the compounds of the present disclosure that are, within the scope of sound medical judgment, suitable for use in contact with the tissues of humans and lower animals without undue toxicity, irritation, allergic response, commensurate with a reasonable benefit/risk ratio, and effective for their intended use, as well as the zwitterionic forms, where possible, of the compounds of the present disclosure. A discussion is provided in Higuchi et al., “Prodrugs as Novel Delivery Systems,” ACS Symposium Series, Vol. 14, and in Roche, E. B., ed. Bioreversible Carriers in Drug Design, American Pharmaceutical Association and Pergamon Press, 1987, both of which are incorporated herein by reference.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, malate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

As used herein, “a pharmaceutically acceptable salt” and/or “deuterated derivative thereof” is intended to encompass pharmaceutically acceptable salts of any one of the referenced compounds, deuterated derivatives of any one of the referenced compounds, and/or pharmaceutically acceptable salts of those deuterated derivatives.

As used herein, nomenclature for compounds including organic compounds, can be given using common names, IUPAC, IUBMB, or CAS recommendations for nomenclature. One of skill in the art can readily ascertain the structure of a compound if given a name, either by systemic reduction of compound structure using naming conventions, or by commercially available software, such as CHEMDRAW™ (Cambridgesoft Corporation, U.S.A.). Chemical names were generated using PerkinElmer ChemDraw® Professional, version 17.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers, or diastereomers. These compounds may be designated by the symbols “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. In some embodiments, an enantiomer or stereoisomer may be provided substantially free of the corresponding enantiomer.

As used herein, “cancer” refers to diseases, disorders, and/or conditions that involve abnormal cell growth with the potential to invade or spread to other parts of the body. Exemplary cancers include, but are not limited to, breast cancer, lung cancer, ovarian cancer, endometrial cancer, prostate cancer, and esophageal cancer.

“Subject” refers to an animal, such as a mammal, that has been or will be the object of treatment, observation, or experiment. The methods described herein may be useful for both human therapy and veterinary applications. In one embodiment, the subject refers to, for example, primates (e.g., humans, male or female), cows, sheep, goats, horses, dogs, cats, rabbits, rats, mice, fish, birds, and the like. In some embodiments, the subject is a primate. In some embodiments, the subject is a human.

As used herein, the term “inhibit,” “inhibition,” or “inhibiting” refers to the reduction or suppression of a given condition, symptom, disorder, disease, or a significant decrease in the baseline activity of a biological activity or process.

As used herein, the term “treat,” “treating,” or “treatment” of any disease or disorder refers in one embodiment to ameliorating the disease or disorder (i.e., slowing or arresting or reducing the development of the disease or at least one of the clinical symptoms thereof). In another embodiment “treat,” “treating,” or “treatment” refers to alleviating or ameliorating at least one physical parameter including those which may not be discernible by the patient. In yet another embodiment, “treat,” “treating,” or “treatment” refers to modulating the disease or disorder, either physically (e.g., through stabilization of a discernible symptom), physiologically, (e.g., through stabilization of a physical parameter), or both. In yet another embodiment, “treat,” “treating,” or “treatment” refers to preventing or delaying the onset or development or progression of the disease or disorder.

As used herein, a subject is “in need of” a treatment if such subject would benefit biologically, medically or in quality of life from such treatment.

### Compounds

In some embodiments, provided herein are compounds of Formula (I), or a tautomer, stereoisomer or a mixture of stereoisomers, a pharmaceutically acceptable salt, hydrate, or deuterated derivative thereof:

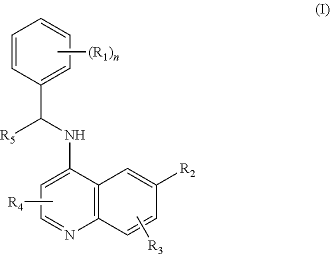

wherein:
R₁ is independently chosen from C₁-C₅ alkyl, halogen, C₁-C₅ alkoxy, C₁-C₄ haloalkyl, and cyano, and n is 0, 1, or 2;
R₂ is chosen from a 4- to 10-membered heteromonocyclic group, fused heterocyclic group, and bridged or spiro heterocyclic group, wherein each of the heteromonocyclic group, fused heterocyclic group, and bridged or spiro heterocyclic group is independently substituted with 0, 1, 2, or 3 R^{a};
R₃ is absent or each R₃ is alkoxyl, C₁-C₅ alkyl, or halo group;
R₄ is absent or each R₄ is cyano or alkyl;
R₅ is a C₁-C₄ alkyl;
R^{a} is absent or each R^{a} is independently chosen from a hydrogen, alkoxyl group, deuterated alkoxyl group, deuterated alkyl group, hydroxyl, halo, haloalkyl, alkyl, and 4- to 5-member heterocycle which is independently substituted with 0 or 1 R₆, C(O)R₇, C(O)NR₇, N(R₇)₂, NR₇C(O)R₇, NR₇C(O)OR₇, or C(O)OR₇ group;
R₆ is absent or alkyl; and
each R₇ is independently chosen from hydrogen, alkoxyl, N(CH₃)₂, NHCH₃, a 3- to 6-member cycloalkyl, a 3- to 6-member heterocycle, C₁-C₅ alkyl, CH₂—CN, and haloalkyl.

In some embodiments, R₂ is chosen from

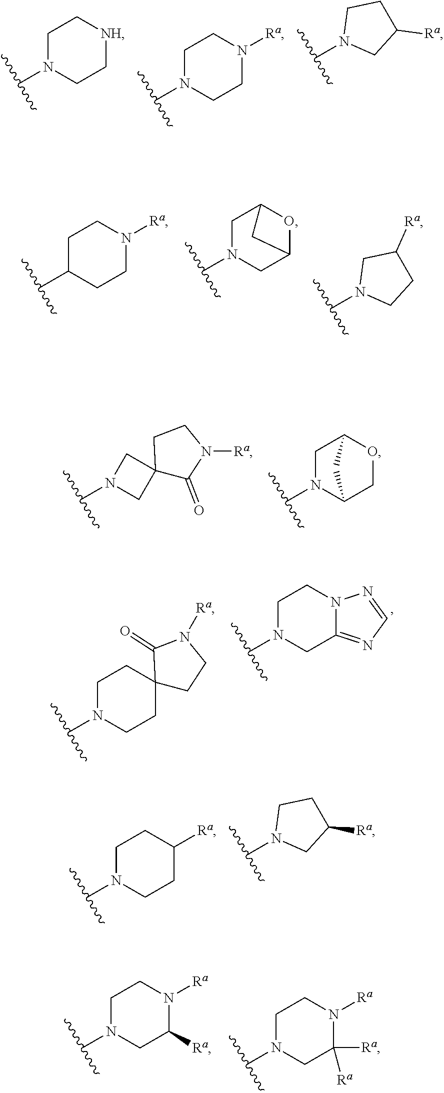

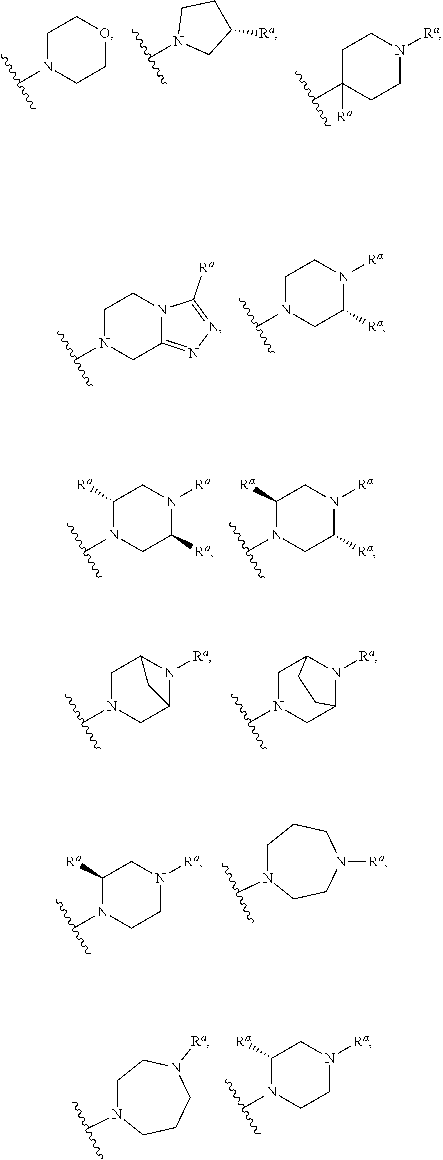

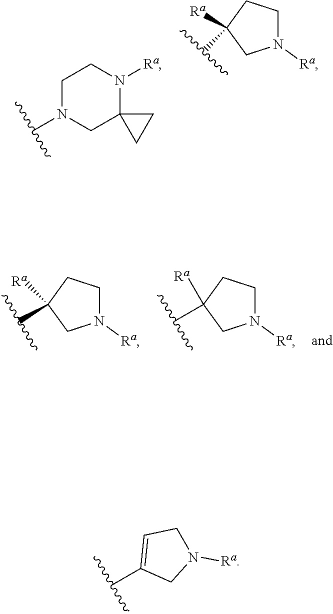

In some embodiments, R^{a} is a 4- to 5-member heterocycle which is independently substituted with 0 or 1 R₆. In some embodiments, R^{a} is chosen from

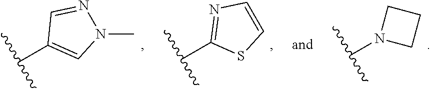

In some embodiments, R^{a} is haloalkyl. In some embodiments, R^{a} is.

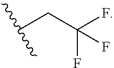 [SMILES: *CC(F)(F)F]

In some embodiments, R^{a} is alkyl. In some embodiments, R^{a} is methyl. In some embodiments, R^{a} is alkoxyl. In some embodiments, R^{a} is

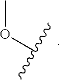 [SMILES: *OC]

In some embodiments, R^{a} is halo. In some embodiments, R^{a} is chosen from C₁, Br, and F. In some embodiments, R^{a} is chosen from C(O)R₇, C(O)NR₇, N(R₇)₂, NR₇C(O)R₇, NR₇C(O)OR₇, and C(O)OR₇. In some embodiments, R₆ is C₁-C₃ alkyl. In some embodiments, R₆ is chosen from methyl, ethyl, propyl, and isopropyl. In some embodiments, R₇ is a 3- to 5-member cycloalkyl or a 3- to 6-member heterocycle. In some embodiments, R₇ is chosen from

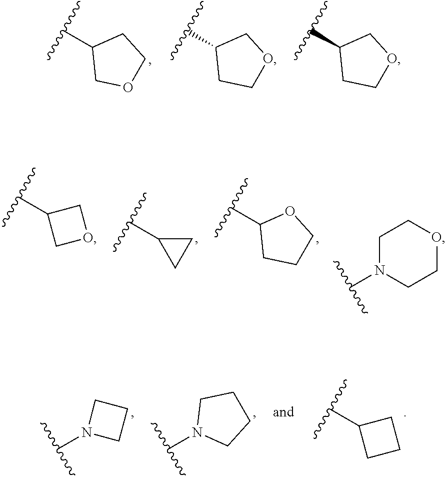

In some embodiments, R₇ is hydrogen. In some embodiments, R₇ is hydrogen or alkyl. In some embodiments, R₇ is hydrogen or methyl. In some embodiments, R₇ is hydrogen or

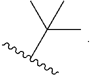 [SMILES: *C(C)(C)C]

In some embodiments, R₇ is hydrogen or

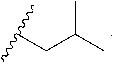 [SMILES: *CC(C)C]

In some embodiments, R₇ is methyl. In some embodiments, R₇ is

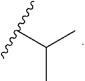 [SMILES: *C(C)C]

In some embodiments, R₇ is C₁-C₃ alkyl or

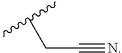 [SMILES: *CC#N]

In some embodiments, R₇ is

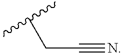 [SMILES: *CC#N]

In some embodiments, R₇ is haloalkyl. In some embodiments, R₇ is

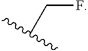 [SMILES: *CF]

In some embodiments, R₃ is absent. In some embodiments, R₃ is alkoxyl. In some embodiments, R₃ is

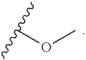 [SMILES: *OC]

In some embodiments, R₃ is halo. In some embodiments, R₃ is F.

In some embodiments, R₁ is haloalkyl or alkyl. In some embodiments, R₅ is a C₁-C₃ alkyl. In some embodiments, R₅ is —CH₃. In some embodiments, R₁ is —CF₃ or methyl. In some embodiments, R₁ is haloalkyl or halogen. In some embodiments, R₁ is —CHF₂ or F. In some embodiments, R₁ is cyano or alkyl. In some embodiments, R₁ is cyano or methyl.

In some embodiments, R₄ is absent. In some embodiments, R₄ is cyano. In some embodiments, R₄ is methyl.

In some embodiments, R₅ is a C₁-C₃ alkyl. In some embodiments, R₅ is —CH₃ and a carbon in Formula (I) to which R₅ is directly attached has an (R)- or (S)-configuration, as shown below in Formula IA:

### R or S Configuration

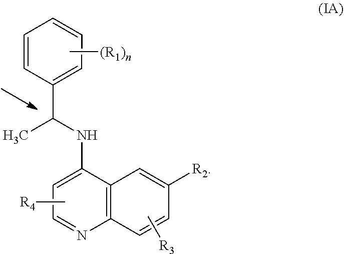

In some embodiments, the carbon in Formula (I) to which R₅ is directly attached has the (R)-configuration.

In some embodiments, provided herein is a pharmaceutically acceptable salt of a compound of Formula (I). In some embodiments, provided herein is a deuterated derivative of a pharmaceutically acceptable salt of a compound of Formula (I). In some embodiments, provided herein is a compound of Formula (I).

In some embodiments, provided herein is a compound chosen from the compounds listed in Table 1 or a tautomer, stereoisomer or a mixture of stereoisomers, a pharmaceutically acceptable salt, a hydrate, or deuterated derivative of any of the foregoing.

TABLE 1
Exemplary Compounds of the Present Disclosure

| Cpd # | Structure | Name |
|---|---|---|
| 1 | 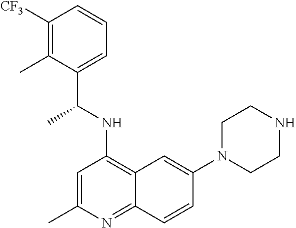 [SMILES: Cc1cc(N[C@H](C)c2cccc(C(F)(F)F)c2C)c2cc(N3CCNCC3)ccc2n1] | (R)-2-methyl-N-(1-(2-methyl-3- (trifluoromethyl)phenyl)ethyl)-6-(piperazin-1- yl)quinolin-4-amine |
| 2 | 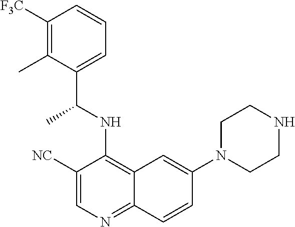 [SMILES: Cc1c([C@@H](C)Nc2c(C#N)cnc3ccc(N4CCNCC4)cc23)cccc1C(F)(F)F] | (R)-4-((1-(2-methyl-3- (trifluoromethyl)phenyl)ethyl)amino)-6- (piperazin-1-yl)quinoline-3-carbonitrile |
| 3 | 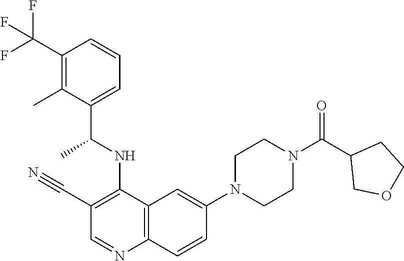 [SMILES: Cc1c([C@@H](C)Nc2c(C#N)cnc3ccc(N4CCN(C(=O)C5CCOC5)CC4)cc23)cccc1C(F)(F)F] | 4-(((R)-1-(2-methyl-3- (trifluoromethyl)phenyl)ethyl)amino)-6-(4- (tetrahydrofuran-3-carbonyl)piperazin-1- yl)quinoline-3-carbonitrile |
| 4 | 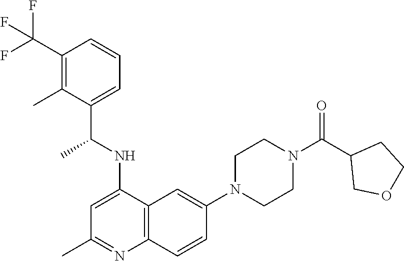 [SMILES: Cc1cc(N[C@H](C)c2cccc(C(F)(F)F)c2C)c2cc(N3CCN(C(=O)C4CCOC4)CC3)ccc2n1] | (4-(2-methyl-4-(((R)-1-(2-methyl-3- (trifluoromethyl)phenyl)ethyl)amino)quinolin-6- yl)piperazin-1-yl)(tetrahydrofuran-3- yl)methanone |
| 5 | 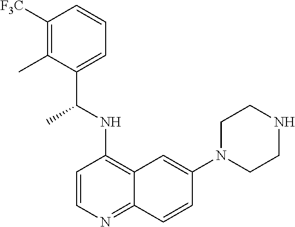 [SMILES: Cc1c([C@@H](C)Nc2ccnc3ccc(N4CCNCC4)cc23)cccc1C(F)(F)F] | (R)-N-(1-(2-methyl-3- (trifluoromethyl)phenyl)ethyl)-6-(piperazin-1- yl)quinolin-4-amine |
| 6 | 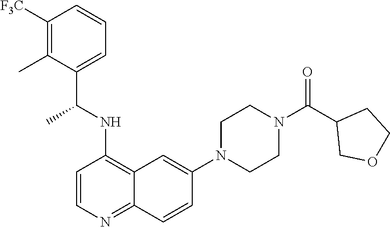 [SMILES: Cc1c([C@@H](C)Nc2ccnc3ccc(N4CCN(C(=O)C5CCOC5)CC4)cc23)cccc1C(F)(F)F] | (4-(4-(((R)-1-(2-methyl-3- (trifluoromethyl)phenyl)ethyl)amino)quinolin-6- yl)piperazin-1-yl)(tetrahydrofuran-3- yl)methanone |
| 7 | 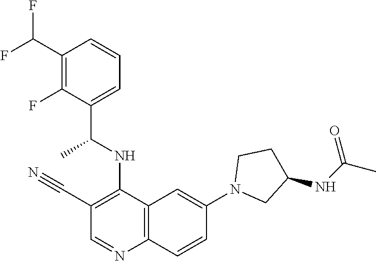 [SMILES: CC(=O)N[C@@H]1CCN(c2ccc3ncc(C#N)c(N[C@H](C)c4cccc(C(F)F)c4F)c3c2)C1] | N-((R)-1-(3-cyano-4-(((R)-1-(3-(difluoromethyl)- 2-fluorophenyl)ethyl)amino)quinolin-6- yl)pyrrolidin-3-yl)acetamide |
| 8 | 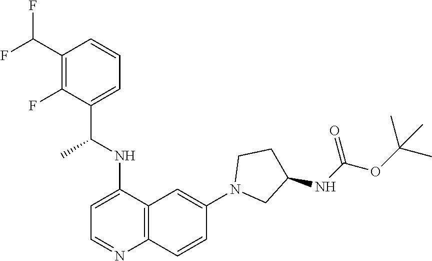 [SMILES: C[C@@H](Nc1ccnc2ccc(N3CC[C@@H](NC(=O)OC(C)(C)C)C3)cc12)c1cccc(C(F)F)c1F] | tert-butyl ((R)-1-(4-(((R)-1-(3-(difluoromethyl)- 2-fluorophenyl)ethyl)amino)quinolin-6- yl)pyrrolidin-3-yl)carbamate |
| 9 | 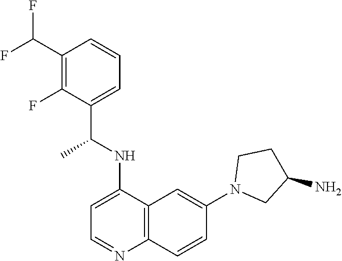 [SMILES: C[C@@H](Nc1ccnc2ccc(N3CC[C@@H](N)C3)cc12)c1cccc(C(F)F)c1F] | 6-((R)-3-aminopyrrolidin-1-yl)-N-((R)-1-(3- (difluoromethyl)-2-fluorophenyl)ethyl)quinolin- 4-amine |
| 10 | 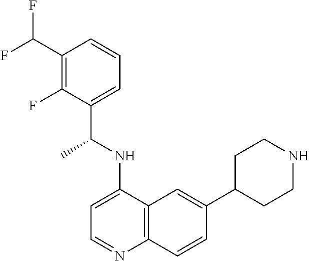 [SMILES: C[C@@H](Nc1ccnc2ccc(C3CCNCC3)cc12)c1cccc(C(F)F)c1F] | (R)-N-(1-(3-(difluoromethyl)-2- fluorophenyl)ethyl)-6-(piperidin-4-yl)quinolin-4- amine |
| 11 | 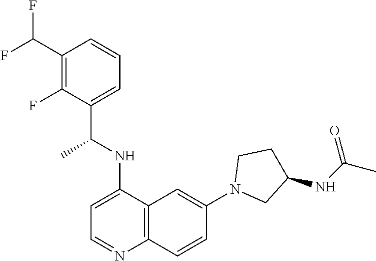 [SMILES: CC(=O)N[C@@H]1CCN(c2ccc3nccc(N[C@H](C)c4cccc(C(F)F)c4F)c3c2)C1] | N-((R)-1-(4-(((R)-1-(3-(difluoromethyl)-2- fluorophenyl)ethyl)amino)quinolin-6- yl)pyrrolidin-3-yl)acetamide |
| 12 | 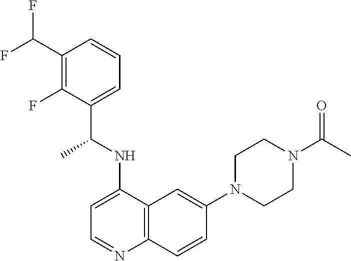 [SMILES: CC(=O)N1CCN(c2ccc3nccc(N[C@H](C)c4cccc(C(F)F)c4F)c3c2)CC1] | (R)-1-(4-(4-((1-(3-(difluoromethyl)-2- fluorophenyl)ethyl)amino)quinolin-6- yl)piperazin-1-yl)ethan-1-one |
| 13 | 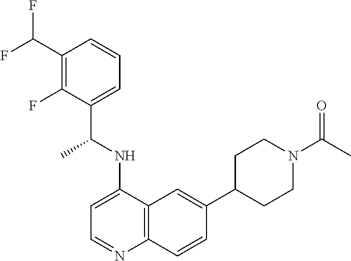 [SMILES: CC(=O)N1CCC(c2ccc3nccc(N[C@H](C)c4cccc(C(F)F)c4F)c3c2)CC1] | (R)-1-(4-(4-((1-(3-(difluoromethyl)-2- fluorophenyl)ethyl)amino)quinolin-6- yl)piperidin-1-yl)ethan-1-one |
| 14 | 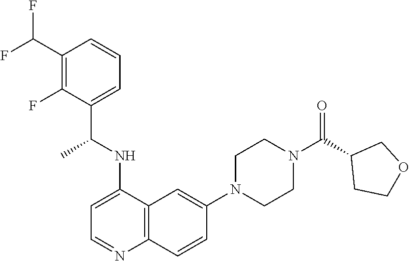 [SMILES: C[C@@H](Nc1ccnc2ccc(N3CCN(C(=O)[C@H]4CCOC4)CC3)cc12)c1cccc(C(F)F)c1F] | (4-(4-(((R)-1-(3-(difluoromethyl)-2- fluorophenyl)ethyl)amino)quinolin-6- yl)piperazin-1-yl)((S)-tetrahydrofuran-3- yl)methanone |
| 15 | 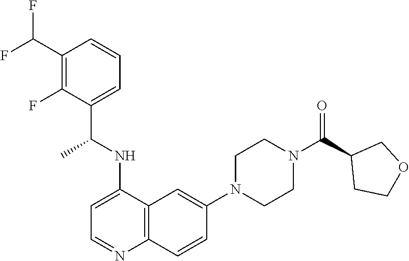 [SMILES: C[C@@H](Nc1ccnc2ccc(N3CCN(C(=O)[C@@H]4CCOC4)CC3)cc12)c1cccc(C(F)F)c1F] | (4-(4-(((R)-1-(3-(difluoromethyl)-2- fluorophenyl)ethyl)amino)quinolin-6- yl)piperazin-1-yl)((R)-tetrahydrofuran-3- yl)methanone |
| 16 | 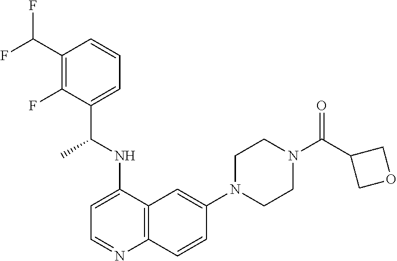 [SMILES: C[C@@H](Nc1ccnc2ccc(N3CCN(C(=O)C4COC4)CC3)cc12)c1cccc(C(F)F)c1F] | (R)-(4-(4-((1-(3-(difluoromethyl)-2- fluorophenyl)ethyl)amino)quinolin-6- yl)piperazin-1-yl)(oxetan-3-yl)methanone |
| 17 | 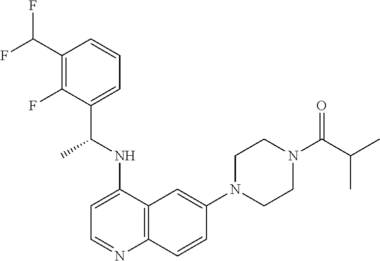 [SMILES: CC(C)C(=O)N1CCN(c2ccc3nccc(N[C@H](C)c4cccc(C(F)F)c4F)c3c2)CC1] | (R)-1-(4-(4-((1-(3-(difluoromethyl)-2- fluorophenyl)ethyl)amino)quinolin-6- yl)piperazin-1-yl)-2-methylpropan-1-one |
| 18 | 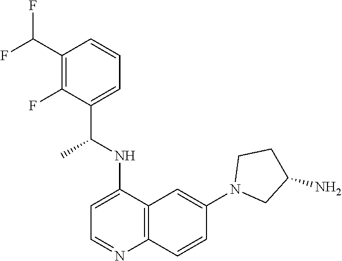 [SMILES: C[C@@H](Nc1ccnc2ccc(N3CC[C@H](N)C3)cc12)c1cccc(C(F)F)c1F] | 6-((S)-3-aminopyrrolidin-1-yl)-N-((R)-1-(3- (difluoromethyl)-2-fluorophenyl)ethyl)quinolin- 4-amine |
| 19 | 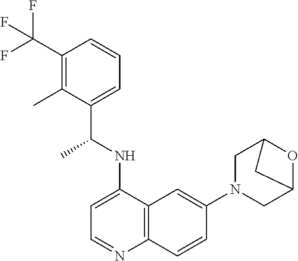 [SMILES: Cc1c([C@@H](C)Nc2ccnc3ccc(N4CC5CC(C4)O5)cc23)cccc1C(F)(F)F] | 6-(6-oxa-3-azabicyclo[3.1.1]heptan-3-yl)-N- ((R)-1-(2-methyl-3- (trifluoromethyl)phenyl)ethyl)quinolin-4-amine |
| 20 | 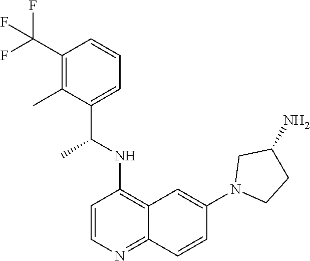 [SMILES: Cc1c([C@@H](C)Nc2ccnc3ccc(N4CC[C@@H](N)C4)cc23)cccc1C(F)(F)F] | 6-((R)-3-aminopyrrolidin-1-yl)-N-((R)-1-(2- methyl-3-(trifluoromethyl)phenyl)ethyl)quinolin- 4-amine |
| 21 | 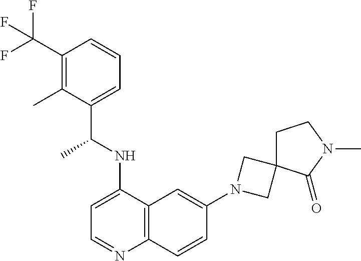 [SMILES: Cc1c([C@@H](C)Nc2ccnc3ccc(N4CC5(CCN(C)C5=O)C4)cc23)cccc1C(F)(F)F] | (R)-6-methyl-2-(4-((1-(2-methyl-3- (trifluoromethyl)phenyl)ethyl)amino)quinolin-6- yl)-2,6-diazaspiro[3.4]octan-5-one |
| 22 | 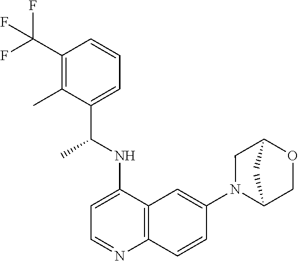 [SMILES: Cc1c([C@@H](C)Nc2ccnc3ccc(N4C[C@@H]5C[C@H]4CO5)cc23)cccc1C(F)(F)F] | 6-((1S,4S)-2-oxa-5-azabicyclo[2.2.1]heptan-5- yl)-N-((R)-1-(2-methyl-3- (trifluoromethyl)phenyl)ethyl)quinolin-4-amine |
| 23 | 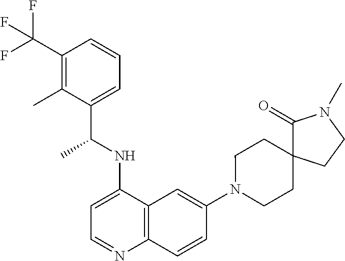 [SMILES: Cc1c([C@@H](C)Nc2ccnc3ccc(N4CCC5(CCN(C)C5=O)CC4)cc23)cccc1C(F)(F)F] | (R)-2-methyl-8-(4-((1-(2-methyl-3- (trifluoromethyl)phenyl)ethyl)amino)quinolin-6- yl)-2,8-diazaspiro[4.5]decan-1-one |
| 24 | 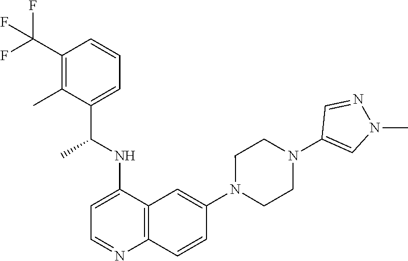 [SMILES: Cc1c([C@@H](C)Nc2ccnc3ccc(N4CCN(c5cnn(C)c5)CC4)cc23)cccc1C(F)(F)F] | (R)-6-(4-(1-methyl-1H-pyrazol-4-yl)piperazin-1- yl)-N-(1-(2-methyl-3- (trifluoromethyl)phenyl)ethyl)quinolin-4-amine |
| 25 | 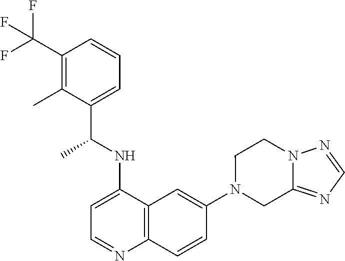 [SMILES: Cc1c([C@@H](C)Nc2ccnc3ccc(N4CCn5ncnc5C4)cc23)cccc1C(F)(F)F] | (R)-6-(5,6-dihydro-[1,2,4]triazolo[1,5-a]pyrazin- 7(8H)-yl)-N-(1-(2-methyl-3- (trifluoromethyl)phenyl)ethyl)quinolin-4-amine |
| 26 | 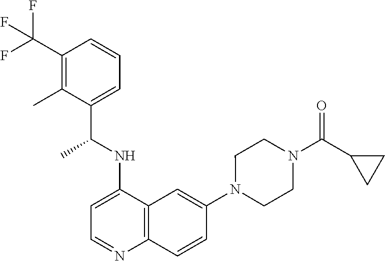 [SMILES: Cc1c([C@@H](C)Nc2ccnc3ccc(N4CCN(C(=O)C5CC5)CC4)cc23)cccc1C(F)(F)F] | (R)-cyclopropyl(4-(4-((1-(2-methyl-3- (trifluoromethyl)phenyl)ethyl)amino)quinolin-6- yl)piperazin-1-yl)methanone |
| 27 | 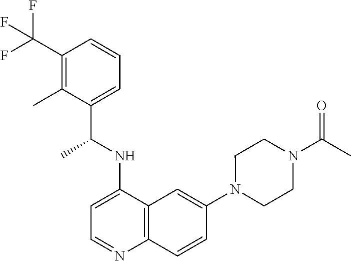 [SMILES: CC(=O)N1CCN(c2ccc3nccc(N[C@H](C)c4cccc(C(F)(F)F)c4C)c3c2)CC1] | (R)-1-(4-(4-((1-(2-methyl-3- (trifluoromethyl)phenyl)ethyl)amino)quinolin-6- yl)piperazin-1-yl)ethan-1-one |
| 28 | 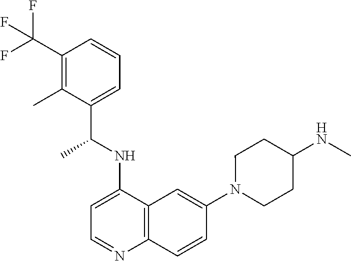 [SMILES: CNC1CCN(c2ccc3nccc(N[C@H](C)c4cccc(C(F)(F)F)c4C)c3c2)CC1] | (R)-N-(1-(2-methyl-3- (trifluoromethyl)phenyl)ethyl)-6-(4- (methylamino)piperidin-1-yl)quinolin-4-amine |
| 29 | 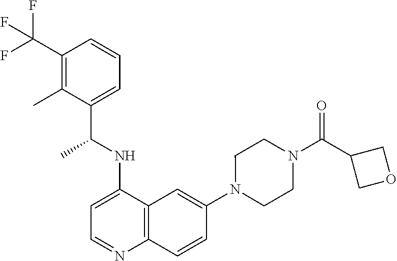 [SMILES: Cc1c([C@@H](C)Nc2ccnc3ccc(N4CCN(C(=O)C5COC5)CC4)cc23)cccc1C(F)(F)F] | (R)-(4-(4-((1-(2-methyl-3- (trifluoromethyl)phenyl)ethyl)amino)quinolin-6- yl)piperazin-1-yl)(oxetan-3-yl)methanone |
| 30 | 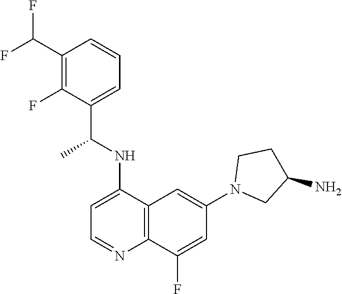 [SMILES: C[C@@H](Nc1ccnc2c(F)cc(N3CC[C@@H](N)C3)cc12)c1cccc(C(F)F)c1F] | 6-((R)-3-aminopyrrolidin-1-yl)-N-((R)-1-(3- (difluoromethyl)-2-fluorophenyl)ethyl)-8- fluoroquinolin-4-amine |
| 31 | 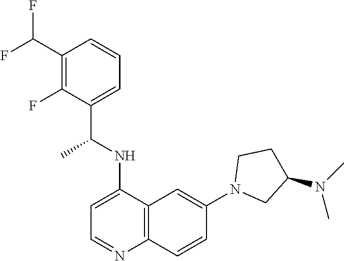 [SMILES: C[C@@H](Nc1ccnc2ccc(N3CC[C@@H](N(C)C)C3)cc12)c1cccc(C(F)F)c1F] | N-((R)-1-(3-(difluoromethyl)-2- fluorophenyl)ethyl)-6-((R)-3- (dimethylamino)pyrrolidin-1-yl)quinolin-4-amine |
| 32 | 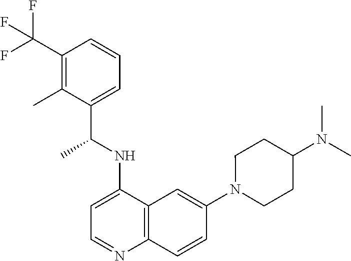 [SMILES: Cc1c([C@@H](C)Nc2ccnc3ccc(N4CCC(N(C)C)CC4)cc23)cccc1C(F)(F)F] | (R)-6-(4-(dimethylamino)piperidin-1-yl)-N-(1-(2- methyl-3-(trifluoromethyl)phenyl)ethyl)quinolin- 4-amine |
| 33 | 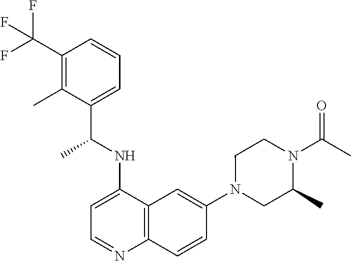 [SMILES: CC(=O)N1CCN(c2ccc3nccc(N[C@H](C)c4cccc(C(F)(F)F)c4C)c3c2)C[C@@H]1C] | 1-((S)-2-methyl-4-(4-(((R)-1-(2-methyl-3- (trifluoromethyl)phenyl)ethyl)amino)quinolin-6- yl)piperazin-1-yl)ethan-1-one |
| 34 | 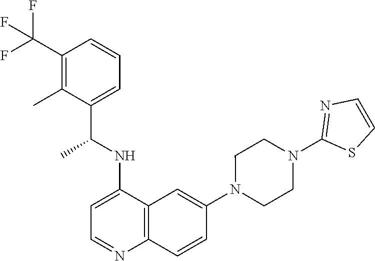 [SMILES: Cc1c([C@@H](C)Nc2ccnc3ccc(N4CCN(c5nccs5)CC4)cc23)cccc1C(F)(F)F] | (R)-N-(1-(2-methyl-3- (trifluoromethyl)phenyl)ethyl)-6-(4-(thiazol-2- yl)piperazin-1-yl)quinolin-4-amine |
| 35 | 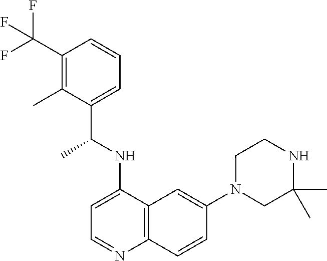 [SMILES: Cc1c([C@@H](C)Nc2ccnc3ccc(N4CCNC(C)(C)C4)cc23)cccc1C(F)(F)F] | (R)-6-(3,3-dimethylpiperazin-1-yl)-N-(1-(2- methyl-2-(trifluoromethyl)phenyl)ethyl)quinolin- 4-amine |
| 36 | 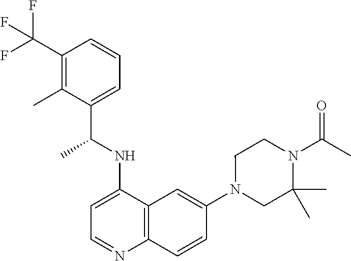 [SMILES: CC(=O)N1CCN(c2ccc3nccc(N[C@H](C)c4cccc(C(F)(F)F)c4C)c3c2)CC1(C)C] | (R)-1-(2,2-dimethyl-4-(4-((1-(2-methyl-3- (trifluoromethyl)phenyl)ethyl)amino)quinolin-6- yl)piperazin-1-yl)ethan-1-one |
| 37 | 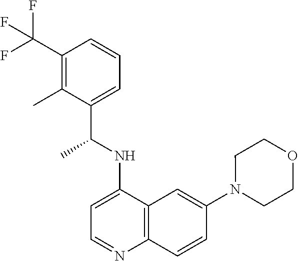 [SMILES: Cc1c([C@@H](C)Nc2ccnc3ccc(N4CCOCC4)cc23)cccc1C(F)(F)F] | (R)-N-(1-(2-methyl-3- (trifluoromethyl)phenyl)ethyl)-6- morpholinoquinolin-4-amine |
| 38 | 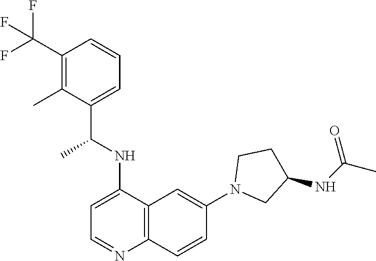 [SMILES: CC(=O)N[C@@H]1CCN(c2ccc3nccc(N[C@H](C)c4cccc(C(F)(F)F)c4C)c3c2)C1] | N-((R)-1-(4-(((R)-1-(2-methyl-3- (trifluoromethyl)phenyl)ethyl)amino)quinolin-6- yl)pyrrolidin-3-yl)acetamide |
| 39 | 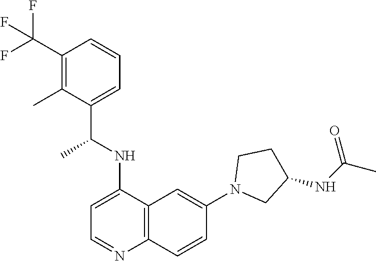 [SMILES: CC(=O)N[C@H]1CCN(c2ccc3nccc(N[C@H](C)c4cccc(C(F)(F)F)c4C)c3c2)C1] | N-((S)-1-(4-(((R)-1-(2-methyl-3- (trifluoromethyl)phenyl)ethyl)amino)quinolin-6- yl)pyrrolidin-3-yl)acetamide |
| 40 | 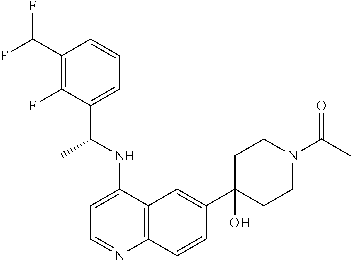 [SMILES: CC(=O)N1CCC(O)(c2ccc3nccc(N[C@H](C)c4cccc(C(F)F)c4F)c3c2)CC1] | (R)-1-(4-(4-((1-(3-(difluoromethyl)-2- fluorophenyl)ethyl)amino)quinolin-6-yl)-4- hydroxypiperidin-1-yl)ethan-1-one |
| 41 | 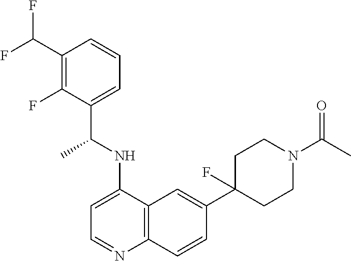 [SMILES: CC(=O)N1CCC(F)(c2ccc3nccc(N[C@H](C)c4cccc(C(F)F)c4F)c3c2)CC1] | (R)-1-(4-(4-((1-(3-(difluoromethyl)-2- fluorophenyl)ethyl)amino)quinolin-6-yl)-4- fluoropiperidin-1-yl)ethan-1-one |
| 42 | 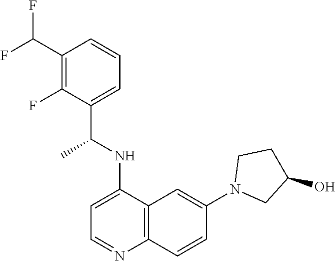 [SMILES: C[C@@H](Nc1ccnc2ccc(N3CC[C@@H](O)C3)cc12)c1cccc(C(F)F)c1F] | (R)-1-(4-(((R)-1-(3-(difluoromethyl)-2- fluorophenyl)ethyl)amino)quinolin-6- yl)pyrrolidin-3-ol |
| 43 | 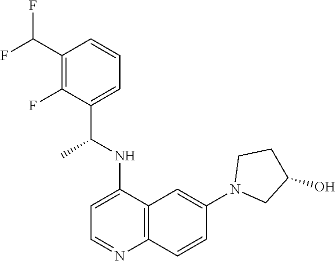 [SMILES: C[C@@H](Nc1ccnc2ccc(N3CC[C@H](O)C3)cc12)c1cccc(C(F)F)c1F] | (S)-1-(4-(((R)-1-(3-(difluoromethyl)-2- fluorophenyl)ethyl)amino)quinolin-6- yl)pyrrolidin-3-ol |
| 44 | 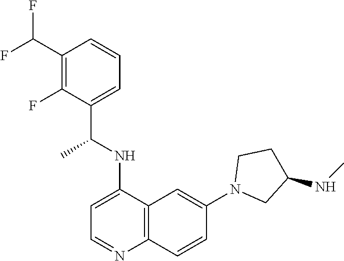 [SMILES: CN[C@@H]1CCN(c2ccc3nccc(N[C@H](C)c4cccc(C(F)F)c4F)c3c2)C1] | N-((R)-1-(3-(difluoromethyl)-2- fluorophenyl)ethyl)-6-((R)-3- (methylamino)pyrrolidin-1-yl)quinolin-4-amine |
| 45 | 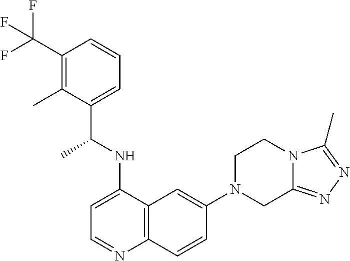 [SMILES: Cc1c([C@@H](C)Nc2ccnc3ccc(N4CCn5c(C)nnc5C4)cc23)cccc1C(F)(F)F] | (R)-N-(1-(2-methyl-3- (trifluoromethyl)phenyl)ethyl)-6-(3-methyl-5,6- dihydro-[1,2,4]triazolo[4,3-a]pyrazin-7(8H)- yl)quinolin-4-amine |
| 46 | 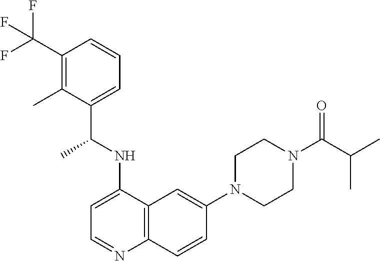 [SMILES: Cc1c([C@@H](C)Nc2ccnc3ccc(N4CCN(C(=O)C(C)C)CC4)cc23)cccc1C(F)(F)F] | (R)-2-methyl-1-(4-(4-((1-(2-methyl-3- (trifluoromethyl)phenyl)ethyl)amino)quinolin-6- yl)piperazin-1-yl)propan-1-one |
| 47 | 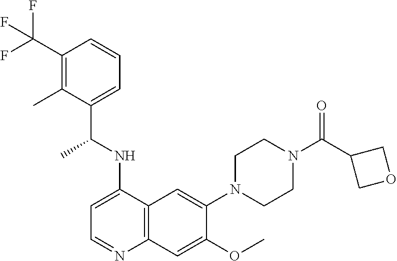 [SMILES: COc1cc2nccc(N[C@H](C)c3cccc(C(F)(F)F)c3C)c2cc1N1CCN(C(=O)C2COC2)CC1] | (R)-4-(7-methoxy-4-((1-(2-methyl-3- (trifluoromethyl)phenyl)ethyl)amino)quinolin-6- yl)piperazin-1-yl)(oxetan-3-yl)methanone |
| 48 | 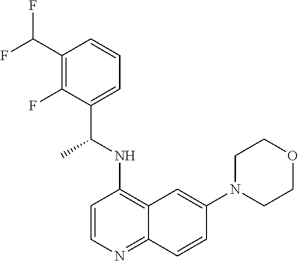 [SMILES: C[C@@H](Nc1ccnc2ccc(N3CCOCC3)cc12)c1cccc(C(F)F)c1F] | (R)-N-(1-(3-(difluoromethyl)-2- fluorophenyl)ethyl)-6-morpholinoquinolin-4- amine |
| 49 | 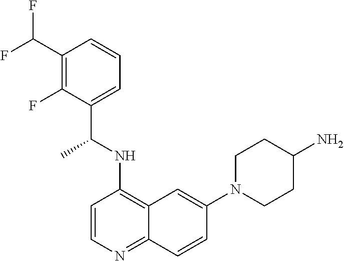 [SMILES: C[C@@H](Nc1ccnc2ccc(N3CCC(N)CC3)cc12)c1cccc(C(F)F)c1F] | (R)-6-(4-aminopiperidin-1-yl)-N-(1-(3- (difluoromethyl)-2-fluorophenyl)ethyl)quinolin- 4-amine |
| 50 | 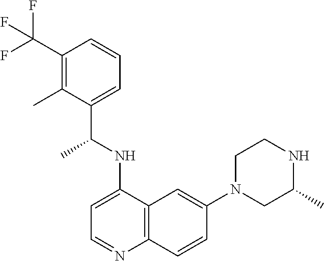 [SMILES: Cc1c([C@@H](C)Nc2ccnc3ccc(N4CCN[C@H](C)C4)cc23)cccc1C(F)(F)F] | N-((R)-1-(2-methyl-3- (trifluoromethyl)phenyl)ethyl)-6-((R)-3- methylpiperazin-1-yl)quinolin-4-amine |
| 51 | 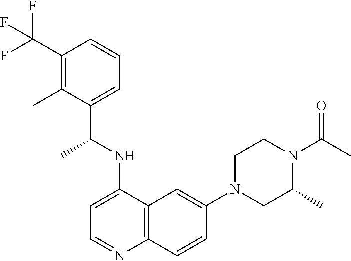 [SMILES: CC(=O)N1CCN(c2ccc3nccc(N[C@H](C)c4cccc(C(F)(F)F)c4C)c3c2)C[C@H]1C] | 1-((R)-2-methyl-4-(4-(((R)-1-(2-methyl-3- (trifluoromethyl)phenyl)ethyl)amino)quinolin-6- yl)piperazin-1-yl)ethan-1-one |
| 52 | 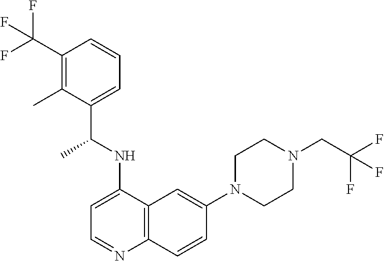 [SMILES: Cc1c([C@@H](C)Nc2ccnc3ccc(N4CCN(CC(F)(F)F)CC4)cc23)cccc1C(F)(F)F] | (R)-N-(1-(2-methyl-3- (trifluoromethyl)phenyl)ethyl)-6-(4-(2,2,2- trifluoroethyl)piperazin-1-yl)quinolin-4-amine |
| 53 | 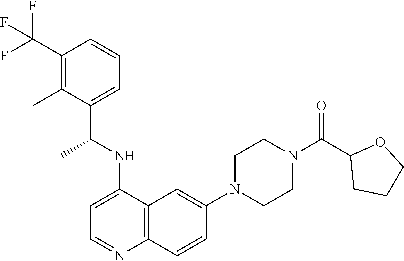 [SMILES: Cc1c([C@@H](C)Nc2ccnc3ccc(N4CCN(C(=O)C5CCCO5)CC4)cc23)cccc1C(F)(F)F] | (4-(4-(((R)-1-(2-methyl-3- (trifluoromethyl)phenyl)ethyl)amino)quinolin-6- yl)piperazin-1-yl)(tetrahydrofuran-2- yl)methanone |
| 54 | 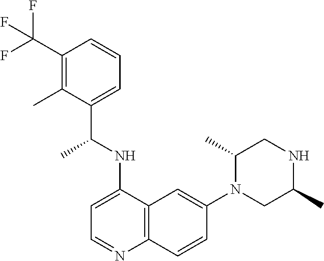 [SMILES: Cc1c([C@@H](C)Nc2ccnc3ccc(N4C[C@H](C)NC[C@H]4C)cc23)cccc1C(F)(F)F] | 6-((2R,5S)-2,5-dimethylpiperazin-1-yl)-N-((R)- 1-(2-methyl-3- (trifluoromethyl)phenyl)ethyl)quinolin-4-amine |
| 55 | 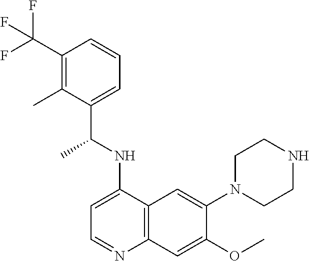 [SMILES: COc1cc2nccc(N[C@H](C)c3cccc(C(F)(F)F)c3C)c2cc1N1CCNCC1] | (R)-7-methoxy-N-(1-(2-methyl-3- (trifluoromethyl)phenyl)ethyl)-6-(piperazin-1- yl)quinolin-4-amine |
| 56 | 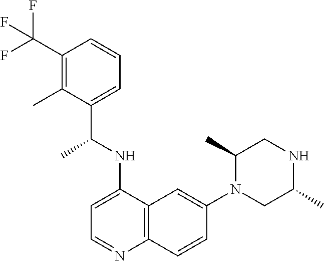 [SMILES: Cc1c([C@@H](C)Nc2ccnc3ccc(N4C[C@@H](C)NC[C@@H]4C)cc23)cccc1C(F)(F)F] | 6-((2S,5R)-2,5-dimethylpiperazin-1-yl)-N-((R)- 1-(2-methyl-3- (trifluoromethyl)phenyl)ethyl)quinolin-4-amine |
| 57 | 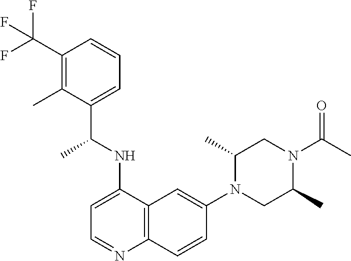 [SMILES: CC(=O)N1C[C@@H](C)N(c2ccc3nccc(N[C@H](C)c4cccc(C(F)(F)F)c4C)c3c2)C[C@@H]1C] | 1-((2S,5R)-2,5-dimethyl-4-(4-(((R)-1-(2-methyl- 3-(trifluoromethyl)phenyl)ethyl)amino)quinolin- 6-yl)piperazin-1-yl)ethan-1-one |
| 58 | 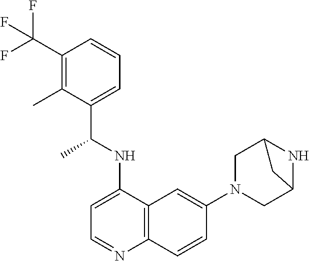 [SMILES: Cc1c([C@@H](C)Nc2ccnc3ccc(N4CC5CC(C4)N5)cc23)cccc1C(F)(F)F] | 6-(3,6-diazabicyclo[3.1.1]heptan-3-yl)-N-((R)-1- (2-methyl-3- (trifluoromethyl)phenyl)ethyl)quinolin-4-amine |
| 59 | 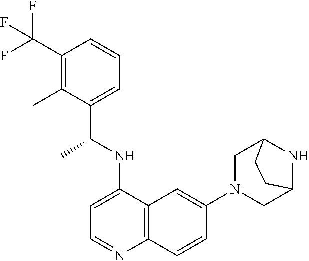 [SMILES: Cc1c([C@@H](C)Nc2ccnc3ccc(N4CC5CCC(C4)N5)cc23)cccc1C(F)(F)F] | 6-(3,8-diazabicyclo[3.2.1]octan-3-yl)-N-((R)-1- (2-methyl-3- (trifluoromethyl)phenyl)ethyl)quinolin-4-amine |
| 60 | 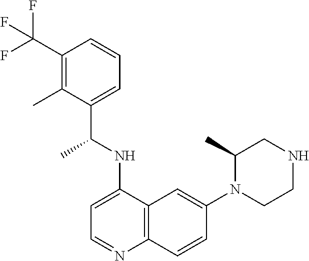 [SMILES: Cc1c([C@@H](C)Nc2ccnc3ccc(N4CCNC[C@@H]4C)cc23)cccc1C(F)(F)F] | N-((R)-1-(2-methyl-3- (trifluoromethyl)phenyl)ethyl)-6-((S)-2- methylpiperazin-1-yl)quinolin-4-amine |
| 61 | 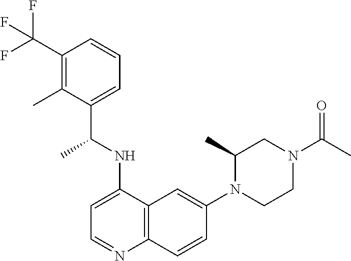 [SMILES: CC(=O)N1CCN(c2ccc3nccc(N[C@H](C)c4cccc(C(F)(F)F)c4C)c3c2)[C@@H](C)C1] | 1-((S)-3-methyl-4-(4-(((R)-1-(2-methyl-3- (trifluoromethyl)phenyl)ethyl)amino)quinolin-6- yl)piperazin-1-yl)ethan-1-one |
| 62 | 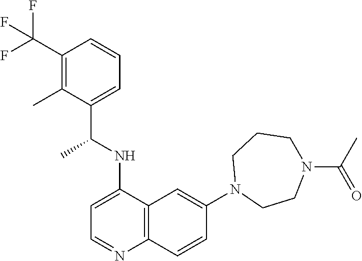 [SMILES: CC(=O)N1CCCN(c2ccc3nccc(N[C@H](C)c4cccc(C(F)(F)F)c4C)c3c2)CC1] | (R)-1-(4-(4-((1-(2-methyl-3- (trifluoromethyl)phenyl)ethyl)amino)quinolin-6- yl)-1,4-diazepan-1-yl)ethan-1-one |
| 63 | 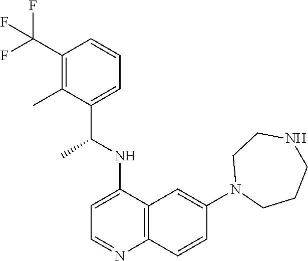 [SMILES: Cc1c([C@@H](C)Nc2ccnc3ccc(N4CCCNCC4)cc23)cccc1C(F)(F)F] | (R)-6-(1,4-diazepan-1-yl)-N-(1-(2-methyl-3- (trifluoromethyl)phenyl)ethyl)quinolin-4-amine |
| 64 | 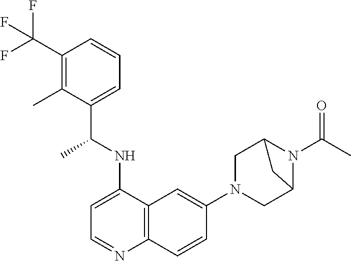 [SMILES: CC(=O)N1C2CC1CN(c1ccc3nccc(N[C@H](C)c4cccc(C(F)(F)F)c4C)c3c1)C2] | 1-(3-(4-(((R)-1-(2-methyl-3- (trifluoromethyl)phenyl)ethyl)amino)quinolin-6- yl)-3,6-diazabicyclo[3.1.1]heptan-6-yl)ethan-1- one |
| 65 | 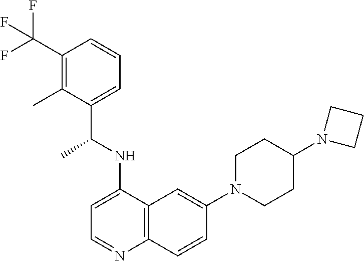 [SMILES: Cc1c([C@@H](C)Nc2ccnc3ccc(N4CCC(N5CCC5)CC4)cc23)cccc1C(F)(F)F] | (R)-6-(4-(azetidin-1-yl)piperidin-1-yl)-N-(1-(2- methyl-3-(trifluoromethyl)phenyl)ethyl)quinolin- 4-amine |
| 66 | 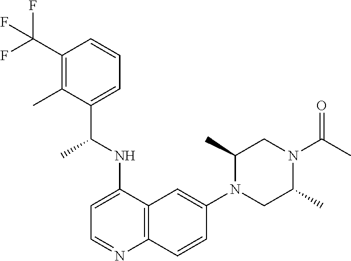 [SMILES: CC(=O)N1C[C@H](C)N(c2ccc3nccc(N[C@H](C)c4cccc(C(F)(F)F)c4C)c3c2)C[C@H]1C] | 1-((2R,5S)-2,5-dimethyl-4-(4-(((R)-1-(2-methyl- 3-(trifluoromethyl)phenyl)ethyl)amino)quinolin- 6-yl)piperazin-1-yl)ethan-1-one |
| 67 | 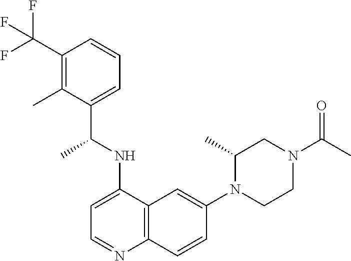 [SMILES: CC(=O)N1CCN(c2ccc3nccc(N[C@H](C)c4cccc(C(F)(F)F)c4C)c3c2)[C@H](C)C1] | 1-((R)-3-methyl-4-(4-(((R)-1-(2-methyl-3- (trifluoromethyl)phenyl)ethyl)amino)quinolin-6- yl)piperazin-1-yl)ethan-1-one |
| 68 | 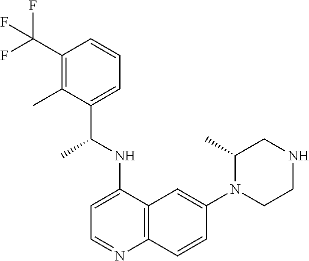 [SMILES: Cc1c([C@@H](C)Nc2ccnc3ccc(N4CCNC[C@H]4C)cc23)cccc1C(F)(F)F] | N-((R)-1-(2-methyl-3- (trifluoromethyl)phenyl)ethyl)-6-((R)-2- methylpiperazin-1-yl)quinolin-4-amine |
| 69 | 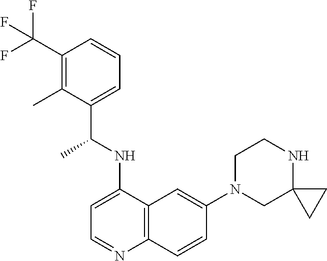 [SMILES: Cc1c([C@@H](C)Nc2ccnc3ccc(N4CCNC5(CC5)C4)cc23)cccc1C(F)(F)F] | (R)-N-(1-(2-methyl-3- (trifluoromethyl)phenyl)ethyl)-6-(4,7- diazaspiro[2.5]octan-7-yl)quinolin-4-amine |
| 70 | 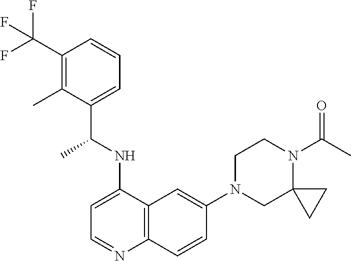 [SMILES: CC(=O)N1CCN(c2ccc3nccc(N[C@H](C)c4cccc(C(F)(F)F)c4C)c3c2)CC12CC2] | (R)-1-(7-(4-((1-(2-methyl-3- (trifluoromethyl)phenyl)ethyl)amino)quinolin-6- yl)-4,7-diazaspiro[2.5]octan-4-yl)ethan-1-one |
| 71 | 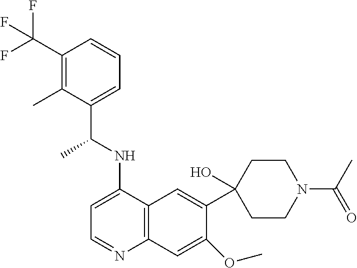 [SMILES: COc1cc2nccc(N[C@H](C)c3cccc(C(F)(F)F)c3C)c2cc1C1(O)CCN(C(C)=O)CC1] | (R)-1-(4-hydroxy-4-(7-methoxy-4-((1-(2-methyl- 3-(trifluoromethyl)phenyl)ethyl)amino)quinolin- 6-yl)piperidin-1-yl)ethan-1-one |
| 72 | 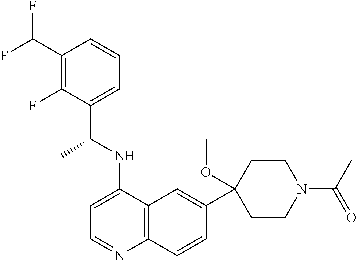 [SMILES: COC1(c2ccc3nccc(N[C@H](C)c4cccc(C(F)F)c4F)c3c2)CCN(C(C)=O)CC1] | (R)-1-(4-(4-((1-(3-(difluoromethyl)-2- fluorophenyl)ethyl)amino)quinolin-6-yl)-4- methoxypiperidin-1-yl)ethan-1-one |
| 73 | 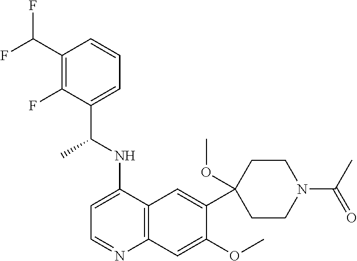 [SMILES: COc1cc2nccc(N[C@H](C)c3cccc(C(F)F)c3F)c2cc1C1(OC)CCN(C(C)=O)CC1] | (R)-1-(4-(4-((1-(3-(difluoromethyl)-2- fluorophenyl)ethyl)amino)-7-methoxyquinolin- 6-yl)-4-methoxypiperidin-1-yl)ethan-1-one |
| 74 | 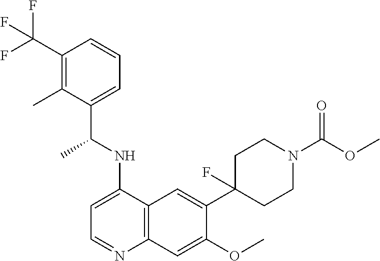 [SMILES: COC(=O)N1CCC(F)(c2cc3c(N[C@H](C)c4cccc(C(F)(F)F)c4C)ccnc3cc2OC)CC1] | Methyl (R)-4-fluoro-4-(7-methoxy-4-((1-(2- methyl-3- (trifluoromethyl)phenyl)ethyl)amino)quinolin-6- yl)piperidine-1-carboxylate |
| 75 | 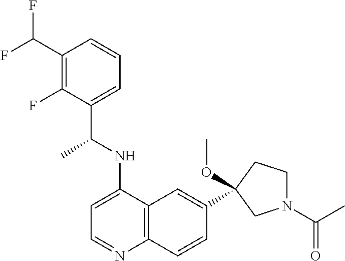 [SMILES: CO[C@@]1(c2ccc3nccc(N[C@H](C)c4cccc(C(F)F)c4F)c3c2)CCN(C(C)=O)C1] | 1-((R)-3-(4-(((R)-1-(3-(difluoromethyl)-2- fluorophenyl)ethyl)amino)quinolin-6-yl)-3- methoxypyrrolidin-1-yl)ethan-1-one |
| 76 | 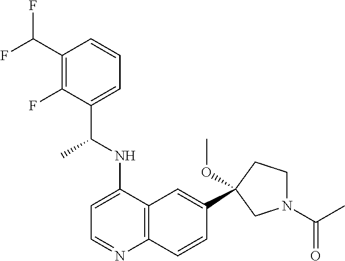 [SMILES: CO[C@]1(c2ccc3nccc(N[C@H](C)c4cccc(C(F)F)c4F)c3c2)CCN(C(C)=O)C1] | 1-((S)-3-(4-(((R)-1-(3-(difluoromethyl)-2- fluorophenyl)ethyl)amino)quinolin-6-yl)-3- methoxypyrrolidin-1-yl)ethan-1-one |
| 77 | 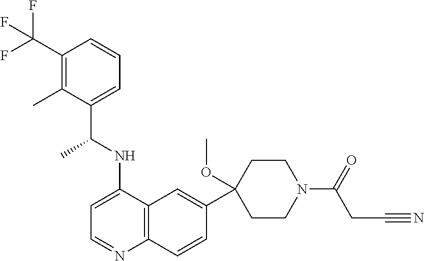 [SMILES: COC1(c2ccc3nccc(N[C@H](C)c4cccc(C(F)(F)F)c4C)c3c2)CCN(C(=O)CC#N)CC1] | (R)-3-(4-methoxy-4-(4-((1-(2-methyl-3- (trifluoromethyl)phenyl)ethyl)amino)quinolin-6- yl)piperidin-1-yl)-3-oxopropanenitrile |
| 78 | 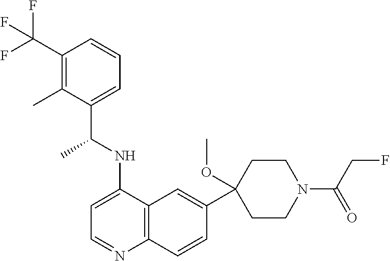 [SMILES: COC1(c2ccc3nccc(N[C@H](C)c4cccc(C(F)(F)F)c4C)c3c2)CCN(C(=O)CF)CC1] | (R)-2-fluoro-1-(4-methoxy-4-(4-((1-(2-methyl-3- (trifluoromethyl)phenyl)ethyl)amino)quinolin-6- yl)piperidin-1-yl)ethan-1-one |
| 79 | 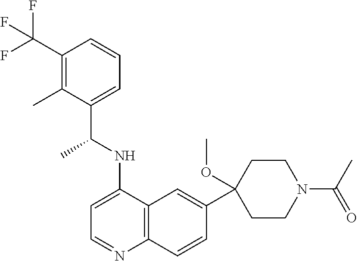 [SMILES: COC1(c2ccc3nccc(N[C@H](C)c4cccc(C(F)(F)F)c4C)c3c2)CCN(C(C)=O)CC1] | (R)-1-(4-methoxy-4-(4-((1-(2-methyl-3- (trifluoromethyl)phenyl)ethyl)amino)quinolin-6- yl)piperidin-1-yl)ethan-1-one |
| 80 | 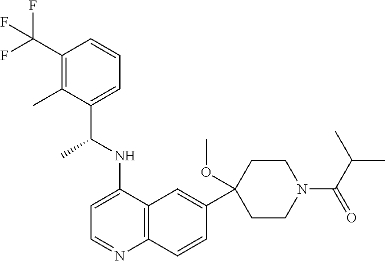 [SMILES: COC1(c2ccc3nccc(N[C@H](C)c4cccc(C(F)(F)F)c4C)c3c2)CCN(C(=O)C(C)C)CC1] | (R)-1-(4-methoxy-4-(4-((1-(2-methyl-3- (trifluoromethyl)phenyl)ethyl)amino)quinolin-6- yl)piperidin-1-yl)-2-methylpropan-1-one |
| 81 | 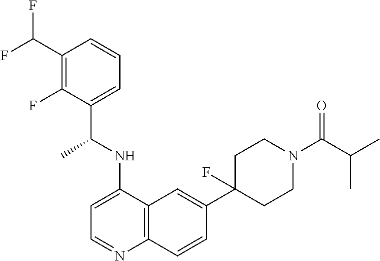 [SMILES: CC(C)C(=O)N1CCC(F)(c2ccc3nccc(N[C@H](C)c4cccc(C(F)F)c4F)c3c2)CC1] | (R)-1-(4-(4-((1-(3-(difluoromethyl)-2- fluorophenyl)ethyl)amino)quinolin-6-yl)-4- fluoropiperidin-1-yl)-2-methylpropan-1-one |
| 82 | 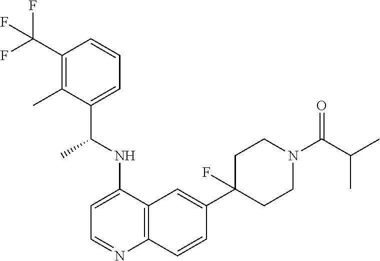 [SMILES: Cc1c([C@@H](C)Nc2ccnc3ccc(C4(F)CCN(C(=O)C(C)C)CC4)cc23)cccc1C(F)(F)F] | (R)-1-(4-fluoro-4-(4-((1-(2-methyl-3- (trifluoromethyl)phenyl)ethyl)amino)quinolin-6- yl)piperidin-1-yl)-2-methylpropan-1-one |
| 83 | 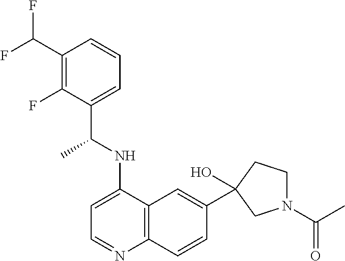 [SMILES: CC(=O)N1CCC(O)(c2ccc3nccc(N[C@H](C)c4cccc(C(F)F)c4F)c3c2)C1] | 1-(3-(4-(((R)-1-(3-(difluoromethyl)-2- fluorophenyl)ethyl)amino)quinolin-6-yl)-3- hydroxypyrrolidin-1-yl)ethan-1-one |
| 84 | 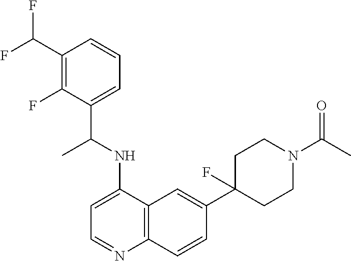 [SMILES: CC(=O)N1CCC(F)(c2ccc3nccc(NC(C)c4cccc(C(F)F)c4F)c3c2)CC1] | 1-(4-(4-((1-(3-(difluoromethyl)-2- fluorophenyl)ethyl)amino)quinolin-6-yl)-4- fluoropiperidin-1-yl)ethan-1-one |
| 85 | 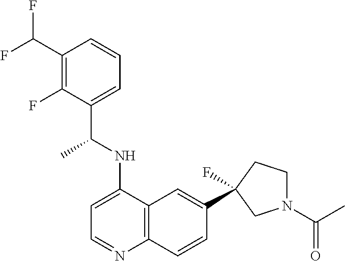 [SMILES: CC(=O)N1CC[C@](F)(c2ccc3nccc(N[C@H](C)c4cccc(C(F)F)c4F)c3c2)C1] | 1-((S)-3-(4-(((R)-1-(3-(difluoromethyl)-2- fluorophenyl)ethyl)amino)quinolin-6-yl)-3- fluoropyrrolidin-1-yl)ethan-1-one |
| 86 | 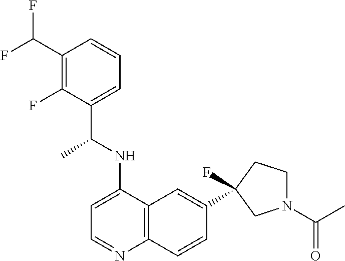 [SMILES: CC(=O)N1CC[C@@](F)(c2ccc3nccc(N[C@H](C)c4cccc(C(F)F)c4F)c3c2)C1] | 1-((R)-3-(4-(((R)-1-(3-(difluoromethyl)-2- fluorophenyl)ethyl)amino)quinolin-6-yl)-3- fluoropyrrolidin-1-yl)ethan-1-one |
| 87 | 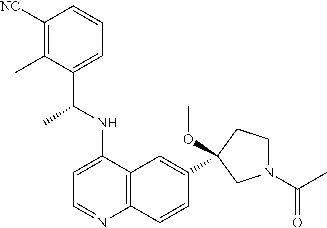 [SMILES: CO[C@@]1(c2ccc3nccc(N[C@H](C)c4cccc(C#N)c4C)c3c2)CCN(C(C)=O)C1] | 3-((R)-1-((6-((R)-1-acetyl-3-methoxypyrrolidin- 3-yl)quinolin-4-yl)amino)ethyl)-2- methylbenzonitrile |
| 88 | 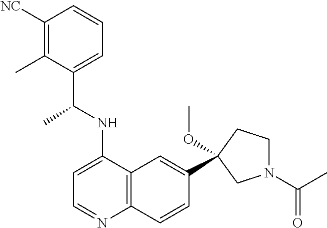 [SMILES: CO[C@]1(c2ccc3nccc(N[C@H](C)c4cccc(C#N)c4C)c3c2)CCN(C(C)=O)C1] | 3-((R)-1-((6-((S)-1-acetyl-3-methoxypyrrolidin- 3-yl)quinolin-4-yl)amino)ethyl)-2- methylbenzonitrile |
| 89 | 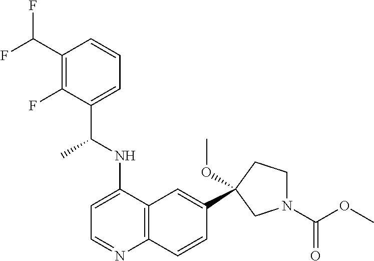 [SMILES: COC(=O)N1CC[C@](OC)(c2ccc3nccc(N[C@H](C)c4cccc(C(F)F)c4F)c3c2)C1] | methyl (S)-3-(4-(((R)-1-(3-(difluoromethyl)-2- fluorophenyl)ethyl)amino)quinolin-6-yl)-3- methoxypyrrolidine-1-carboxylate |
| 90 | 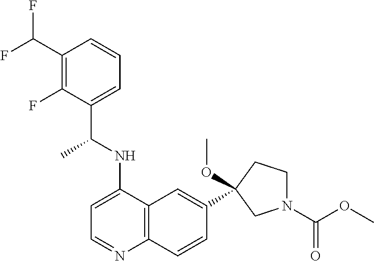 [SMILES: COC(=O)N1CC[C@@](OC)(c2ccc3nccc(N[C@H](C)c4cccc(C(F)F)c4F)c3c2)C1] | methyl (R)-3-(4-(((R)-1-(3-(difluoromethyl)-2- fluorophenyl)ethyl)amino)quinolin-6-yl)-3- methoxypyrrolidine-1-carboxylate |
| 91 | 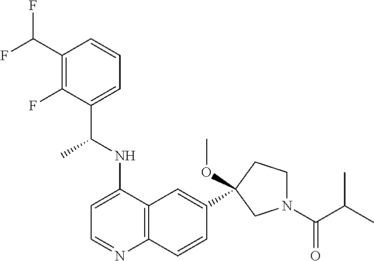 [SMILES: CO[C@@]1(c2ccc3nccc(N[C@H](C)c4cccc(C(F)F)c4F)c3c2)CCN(C(=O)C(C)C)C1] | 1-((R)-3-(4-(((R)-1-(3-(difluoromethyl)-2- fluorophenyl)ethyl)amino)quinolin-6-yl)-3- methoxypyrrolidin-1-yl)-2-methylpropan-1-one |
| 92 | 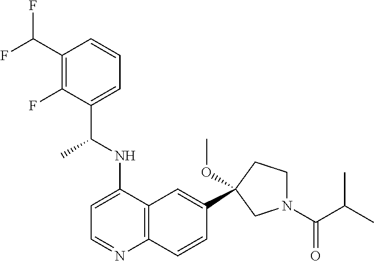 [SMILES: CO[C@]1(c2ccc3nccc(N[C@H](C)c4cccc(C(F)F)c4F)c3c2)CCN(C(=O)C(C)C)C1] | 1-((S)-3-(4-(((R)-1-(3-(difluoromethyl)-2- fluorophenyl)ethyl)amino)quinolin-6-yl)-3- methoxypyrrolidin-1-yl)-2-methylpropan-1-one |
| 93 | 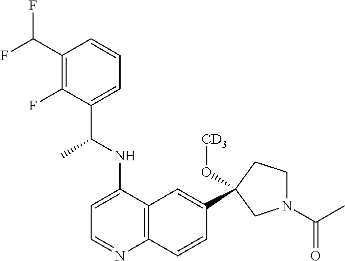 [SMILES: [2H]C([2H])([2H])O[C@]1(c2ccc3nccc(N[C@H](C)c4cccc(C(F)F)c4F)c3c2)CCN(C(C)=O)C1] | 1-((S)-3-(4-(((R)-1-(3-(difluoromethyl)-2- fluorophenyl)ethyl)amino)quinolin-6-yl)-3- (methoxy-d3)pyrrolidin-1-yl)ethan-1-one |
| 94 | 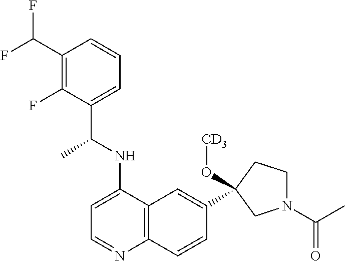 [SMILES: [2H]C([2H])([2H])O[C@@]1(c2ccc3nccc(N[C@H](C)c4cccc(C(F)F)c4F)c3c2)CCN(C(C)=O)C1] | 1-((R)-3-(4-(((R)-1-(3-(difluoromethyl)-2- fluorophenyl)ethyl)amino)quinolin-6-yl)-3- (methoxy-d3)pyrrolidin-1-yl)ethan-1-one |
| 95 | 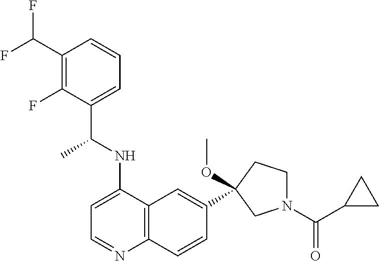 [SMILES: CO[C@@]1(c2ccc3nccc(N[C@H](C)c4cccc(C(F)F)c4F)c3c2)CCN(C(=O)C2CC2)C1] | cyclopropyl((R)-3-(4-(((R)-1-(3- (difluoromethyl)-2- fluorophenyl)ethyl)amino)quinolin-6-yl)-3- methoxypyrrolidin-1-yl)methanone |
| 96 | 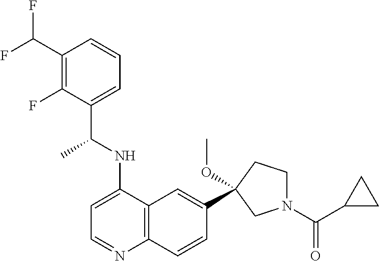 [SMILES: CO[C@]1(c2ccc3nccc(N[C@H](C)c4cccc(C(F)F)c4F)c3c2)CCN(C(=O)C2CC2)C1] | cyclopropyl((S)-3-(4-(((R)-1-(3-(difluoromethyl)- 2-fluorophenyl)ethyl)amino)quinolin-6-yl)-3- methoxypyrrolidin-1-yl)methanone |
| 97 | 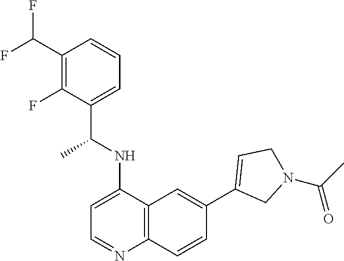 [SMILES: CC(=O)N1CC=C(c2ccc3nccc(N[C@H](C)c4cccc(C(F)F)c4F)c3c2)C1] | (R)-1-(3-(4-((1-(3-(difluoromethyl)-2- fluorophenyl)ethyl)amino)quinolin-6-yl)-2,5- dihydro-1H-pyrrol-1-yl)ethan-1-one |
| 98 | 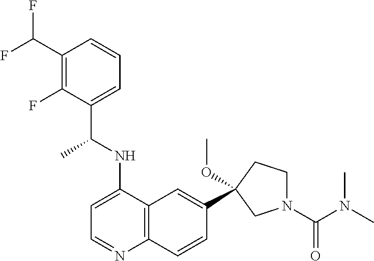 [SMILES: CO[C@]1(c2ccc3nccc(N[C@H](C)c4cccc(C(F)F)c4F)c3c2)CCN(C(=O)N(C)C)C1] | (S)-3-(4-(((R)-1-(3-(difluoromethyl)-2- fluorophenyl)ethyl)amino)quinolin-6-yl)-3- methoxy-N,N-dimethylpyrrolidine-1- carboxamide |
| 99 | 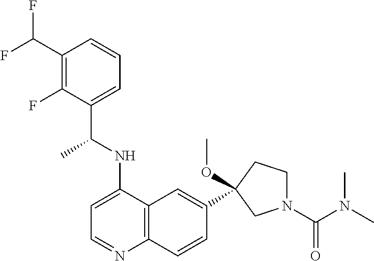 [SMILES: CO[C@@]1(c2ccc3nccc(N[C@H](C)c4cccc(C(F)F)c4F)c3c2)CCN(C(=O)N(C)C)C1] | (R)-3-(4-(((R)-1-(3-(difluoromethyl)-2- fluorophenyl)ethyl)amino)quinolin-6-yl)-3- methoxy-N,N-dimethylpyrrolidine-1- carboxamide |
| 100 | 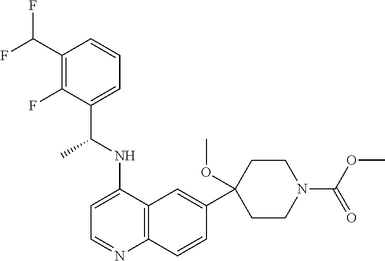 [SMILES: COC(=O)N1CCC(OC)(c2ccc3nccc(N[C@H](C)c4cccc(C(F)F)c4F)c3c2)CC1] | methyl (R)-4-(4-((1-(3-(difluoromethyl)-2- fluorophenyl)ethyl)amino)quinolin-6-yl)-4- methoxypiperidine-1-carboxylate |
| 101 | 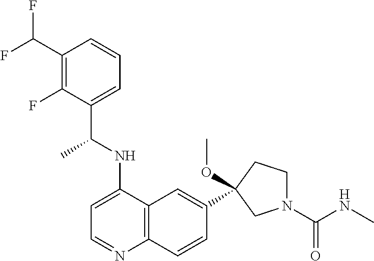 [SMILES: CNC(=O)N1CC[C@@](OC)(c2ccc3nccc(N[C@H](C)c4cccc(C(F)F)c4F)c3c2)C1] | (R)-3-(4-(((R)-1-(3-(difluoromethyl)-2- fluorophenyl)ethyl)amino)quinolin-6-yl)-3- methoxy-N-methylpyrrolidine-1-carboxamide |
| 102 | 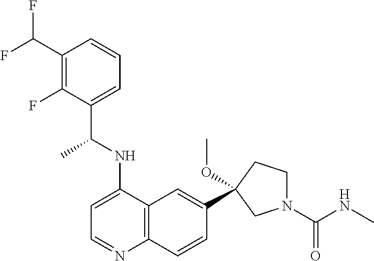 [SMILES: CNC(=O)N1CC[C@](OC)(c2ccc3nccc(N[C@H](C)c4cccc(C(F)F)c4F)c3c2)C1] | (S)-3-(4-(((R)-1-(3-(difluoromethyl)-2- fluorophenyl)ethyl)amino)quinolin-6-yl)-3- methoxy-N-methylpyrrolidine-1-carboxamide |
| 103 | 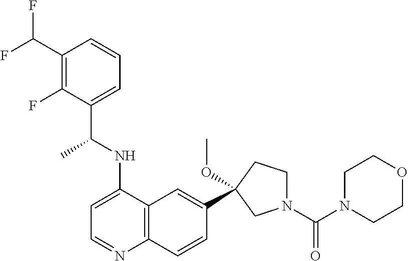 [SMILES: CO[C@]1(c2ccc3nccc(N[C@H](C)c4cccc(C(F)F)c4F)c3c2)CCN(C(=O)N2CCOCC2)C1] | ((S)-3-(4-(((R)-1-(3-(difluoromethyl)-2- fluorophenyl)ethyl)amino)quinolin-6-yl)-3- methoxypyrrolidin-1-yl)(morpholino)methanone |
| 104 | 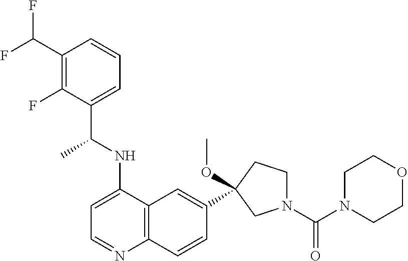 [SMILES: CO[C@@]1(c2ccc3nccc(N[C@H](C)c4cccc(C(F)F)c4F)c3c2)CCN(C(=O)N2CCOCC2)C1] | ((R)-3-(4-(((R)-1-(3-(difluoromethyl)-2- fluorophenyl)ethyl)amino)quinolin-6-yl)-3- methoxypyrrolidin-1-yl)(morpholino)methanone |
| 105 | 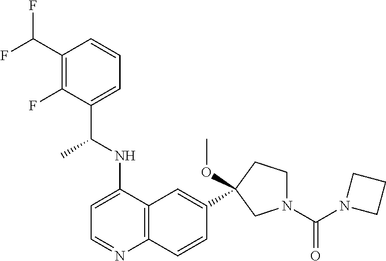 [SMILES: CO[C@@]1(c2ccc3nccc(N[C@H](C)c4cccc(C(F)F)c4F)c3c2)CCN(C(=O)N2CCC2)C1] | azetidin-1-yl((R)-3-(4-(((R)-1-(3- (difluoromethyl)-2- fluorophenyl)ethyl)amino)quinolin-6-yl)-3- methoxypyrrolidin-1-yl)methanone |
| 106 | 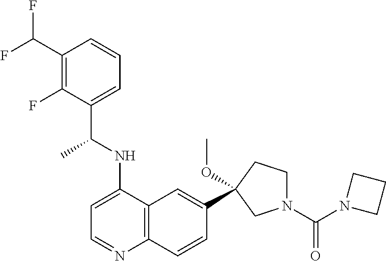 [SMILES: CO[C@]1(c2ccc3nccc(N[C@H](C)c4cccc(C(F)F)c4F)c3c2)CCN(C(=O)N2CCC2)C1] | azetidin-1-yl((S)-3-(4-(((R)-1-(3- (difluoromethyl)-2- fluorophenyl)ethyl)amino)quinolin-6-yl)-3- methoxypyrrolidin-1-yl)methanone |
| 107 | 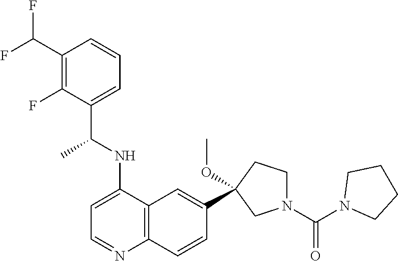 [SMILES: CO[C@]1(c2ccc3nccc(N[C@H](C)c4cccc(C(F)F)c4F)c3c2)CCN(C(=O)N2CCCC2)C1] | ((S)-3-(4-(((R)-1-(3-(difluoromethyl)-2- fluorophenyl)ethyl)amino)quinolin-6-yl)-3- methoxypyrrolidin-1-yl)(pyrrolidin-1- yl)methanone |
| 108 | 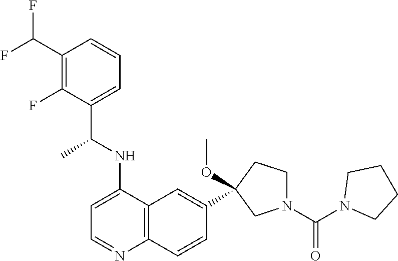 [SMILES: CO[C@@]1(c2ccc3nccc(N[C@H](C)c4cccc(C(F)F)c4F)c3c2)CCN(C(=O)N2CCCC2)C1] | ((R)-3-(4-(((R)-1-(3-(difluoromethyl)-2- fluorophenyl)ethyl)amino)quinolin-6-yl)-3- methoxypyrrolidin-1-yl)(pyrrolidin-1- yl)methanone |
| 109 | 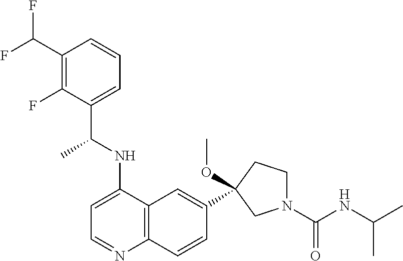 [SMILES: CO[C@@]1(c2ccc3nccc(N[C@H](C)c4cccc(C(F)F)c4F)c3c2)CCN(C(=O)NC(C)C)C1] | (R)-3-(4-(((R)-1-(3-(difluoromethyl)-2- fluorophenyl)ethyl)amino)quinolin-6-yl)-N- isopropyl-3-methoxypyrrolidine-1-carboxamide |
| 110 | 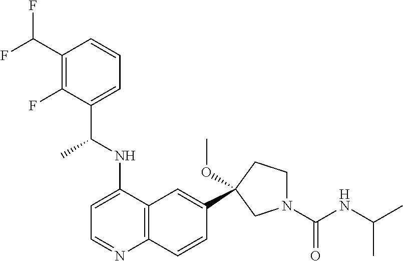 [SMILES: CO[C@]1(c2ccc3nccc(N[C@H](C)c4cccc(C(F)F)c4F)c3c2)CCN(C(=O)NC(C)C)C1] | (S)-3-(4-(((R)-1-(3-(difluoromethyl)-2- fluorophenyl)ethyl)amino)quinolin-6-yl)-N- isopropyl-3-methoxypyrrolidine-1-carboxamide |
| 111 | 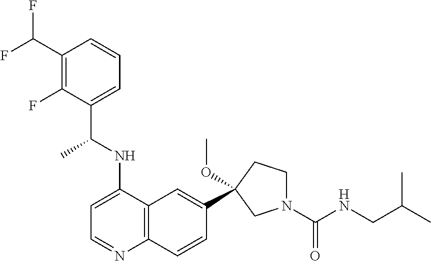 [SMILES: CO[C@]1(c2ccc3nccc(N[C@H](C)c4cccc(C(F)F)c4F)c3c2)CCN(C(=O)NCC(C)C)C1] | (S)-3-(4-(((R)-1-(3-(difluoromethyl)-2- fluorophenyl)ethyl)amino)quinolin-6-yl)-N- isobutyl-3-methoxypyrrolidine-1-carboxamide |
| 112 | 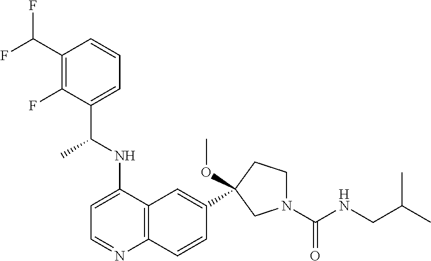 [SMILES: CO[C@@]1(c2ccc3nccc(N[C@H](C)c4cccc(C(F)F)c4F)c3c2)CCN(C(=O)NCC(C)C)C1] | (R)-3-(4-(((R)-1-(3-(difluoromethyl)-2- fluorophenyl)ethyl)amino)quinolin-6-yl)-N- isobutyl-3-methoxypyrrolidine-1-carboxamide |
| 113 | 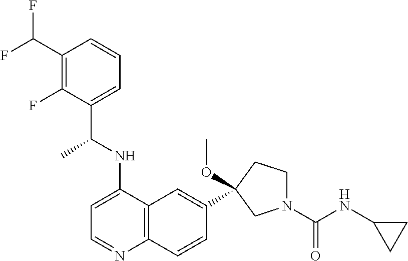 [SMILES: CO[C@@]1(c2ccc3nccc(N[C@H](C)c4cccc(C(F)F)c4F)c3c2)CCN(C(=O)NC2CC2)C1] | (R)-N-cyclopropyl-3-(4-(((R)-1-(3- (difluoromethyl)-2- fluorophenyl)ethyl)amino)quinolin-6-yl)-3- methoxypyrrolidine-1-carboxamide |
| 114 | 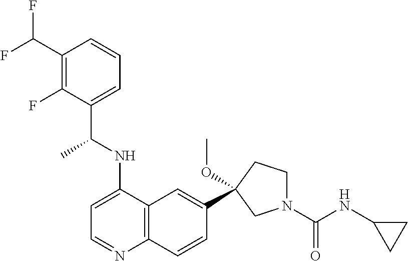 [SMILES: CO[C@]1(c2ccc3nccc(N[C@H](C)c4cccc(C(F)F)c4F)c3c2)CCN(C(=O)NC2CC2)C1] | (S)-N-cyclopropyl-3-(4-(((R)-1-(3- (difluoromethyl)-2- fluorophenyl)ethyl)amino)quinolin-6-yl)-3- methoxypyrrolidine-1-carboxamide |
| 115 | 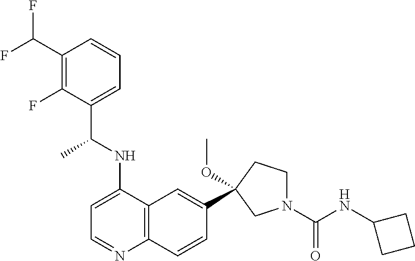 [SMILES: CO[C@]1(c2ccc3nccc(N[C@H](C)c4cccc(C(F)F)c4F)c3c2)CCN(C(=O)NC2CCC2)C1] | (S)-N-cyclobutyl-3-(4-(((R)-1-(3- (difluoromethyl)-2- fluorophenyl)ethyl)amino)quinolin-6-yl)-3- methoxypyrrolidine-1-carboxamide |
| 116 | 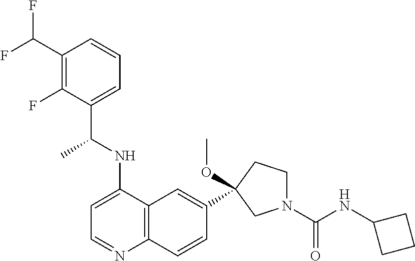 [SMILES: CO[C@@]1(c2ccc3nccc(N[C@H](C)c4cccc(C(F)F)c4F)c3c2)CCN(C(=O)NC2CCC2)C1] | (R)-N-cyclobutyl-3-(4-(((R)-1-(3- (difluoromethyl)-2- fluorophenyl)ethyl)amino)quinolin-6-yl)-3- methoxypyrrolidine-1-carboxamide |
| 117 | 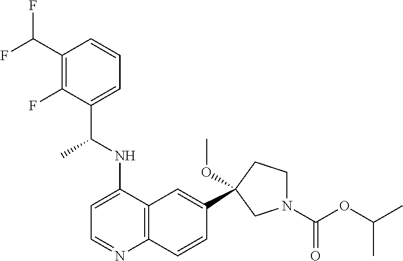 [SMILES: CO[C@]1(c2ccc3nccc(N[C@H](C)c4cccc(C(F)F)c4F)c3c2)CCN(C(=O)OC(C)C)C1] | isopropyl (S)-3-(4-(((R)-1-(3-(difluoromethyl)-2- fluorophenyl)ethyl)amino)quinolin-6-yl)-3- methoxypyrrolidine-1-carboxylate |
| 118 | 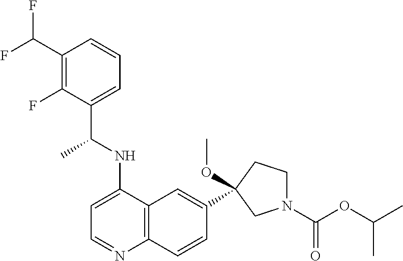 [SMILES: CO[C@@]1(c2ccc3nccc(N[C@H](C)c4cccc(C(F)F)c4F)c3c2)CCN(C(=O)OC(C)C)C1] | isopropyl (R)-3-(4-(((R)-1-(3-(difluoromethyl)-2- fluorophenyl)ethyl)amino)quinolin-6-yl)-3- methoxypyrrolidine-1-carboxylate |
| 119 | 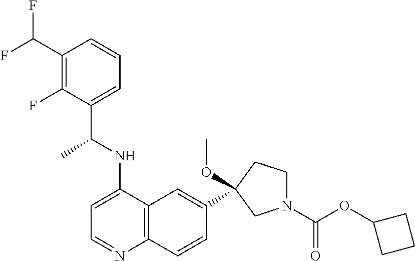 [SMILES: CO[C@@]1(c2ccc3nccc(N[C@H](C)c4cccc(C(F)F)c4F)c3c2)CCN(C(=O)OC2CCC2)C1] | cyclobutyl (R)-3-(4-(((R)-1-(3-(difluoromethyl)- 2-fluorophenyl)ethyl)amino)quinolin-6-yl)-3- methoxypyrrolidine-1-carboxylate |
| 120 | 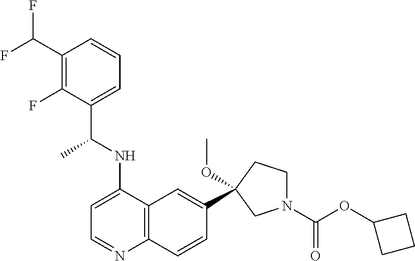 [SMILES: CO[C@]1(c2ccc3nccc(N[C@H](C)c4cccc(C(F)F)c4F)c3c2)CCN(C(=O)OC2CCC2)C1] | cyclobutyl (S)-3-(4-(((R)-1-(3-(difluoromethyl)- 2-fluorophenyl)ethyl)amino)quinolin-6-yl)-3- methoxypyrrolidine-1-carboxylate |
| 121 | 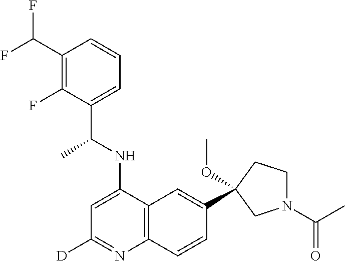 [SMILES: [2H]c1cc(N[C@H](C)c2cccc(C(F)F)c2F)c2cc([C@@]3(OC)CCN(C(C)=O)C3)ccc2n1] | 1-((S)-3-(4-(((R)-1-(3-(difluoromethyl)-2- fluorophenyl)ethyl)amino)quinolin-6-yl-2-d)-3- methoxypyrrolidin-1-yl)ethan-1-one |
| 122 | 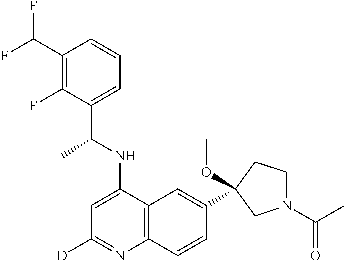 [SMILES: [2H]c1cc(N[C@H](C)c2cccc(C(F)F)c2F)c2cc([C@]3(OC)CCN(C(C)=O)C3)ccc2n1] | 1-((R)-3-(4-(((R)-1-(3-(difluoromethyl)-2- fluorophenyl)ethyl)amino)quinolin-6-yl-2-d)-3- methoxypyrrolidin-1-yl)ethan-1-one |
| 123 | 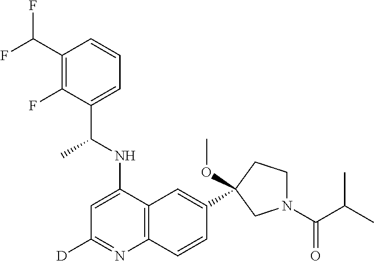 [SMILES: [2H]c1cc(N[C@H](C)c2cccc(C(F)F)c2F)c2cc([C@]3(OC)CCN(C(=O)C(C)C)C3)ccc2n1] | 1-((R)-3-(4-(((R)-1-(3-(difluoromethyl)-2- fluorophenyl)ethyl)amino)quinolin-6-yl-2-d)-3- methoxypyrrolidin-1-yl)-2-methylpropan-1-one |
| 124 | 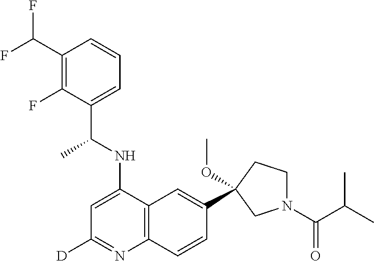 [SMILES: [2H]c1cc(N[C@H](C)c2cccc(C(F)F)c2F)c2cc([C@@]3(OC)CCN(C(=O)C(C)C)C3)ccc2n1] | 1-((S)-3-(4-(((R)-1-(3-(difluoromethyl)-2- fluorophenyl)ethyl)amino)quinolin-6-yl-2-d)-3- methoxypyrrolidin-1-yl)-2-methylpropan-1-one |
| 125 | 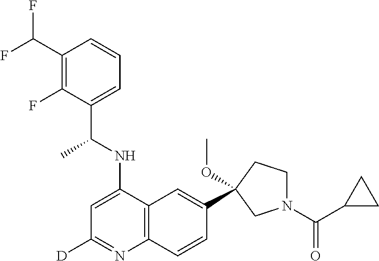 [SMILES: [2H]c1cc(N[C@H](C)c2cccc(C(F)F)c2F)c2cc([C@@]3(OC)CCN(C(=O)C4CC4)C3)ccc2n1] | cyclopropyl((S)-3-(4-(((R)-1-(3-(difluoromethyl)- 2-fluorophenyl)ethyl)amino)quinolin-6-yl-2-d)- 3-methoxypyrrolidin-1-yl)methanone |
| 126 | 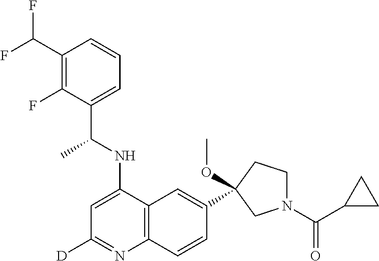 [SMILES: [2H]c1cc(N[C@H](C)c2cccc(C(F)F)c2F)c2cc([C@]3(OC)CCN(C(=O)C4CC4)C3)ccc2n1] | cyclopropyl((R)-3-(4-(((R)-1-(3- (difluoromethyl)-2- fluorophenyl)ethyl)amino)quinolin-6-yl-2-d)-3- methoxypyrrolidin-1-yl)methanone |
| 127 | 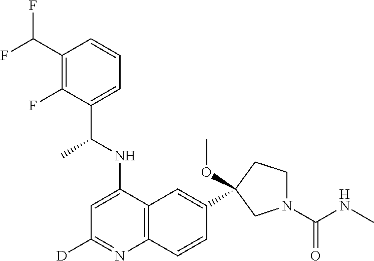 [SMILES: [2H]c1cc(N[C@H](C)c2cccc(C(F)F)c2F)c2cc([C@]3(OC)CCN(C(=O)NC)C3)ccc2n1] | (R)-3-(4-(((R)-1-(3-(difluoromethyl)-2- fluorophenyl)ethyl)amino)quinolin-6-yl-2-d)-3- methoxy-N-methylpyrrolidine-1-carboxamide |
| 128 | 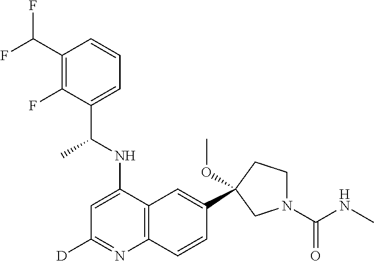 [SMILES: [2H]c1cc(N[C@H](C)c2cccc(C(F)F)c2F)c2cc([C@@]3(OC)CCN(C(=O)NC)C3)ccc2n1] | (S)-3-(4-(((R)-1-(3-(difluoromethyl)-2- fluorophenyl)ethyl)amino)quinolin-6-yl-2-d)-3- methoxy-N-methylpyrrolidine-1-carboxamide |
| 129 | 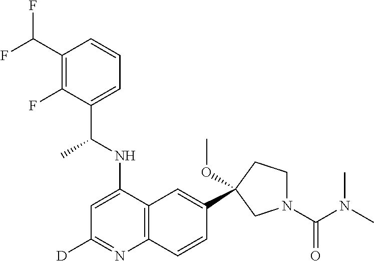 [SMILES: [2H]c1cc(N[C@H](C)c2cccc(C(F)F)c2F)c2cc([C@@]3(OC)CCN(C(=O)N(C)C)C3)ccc2n1] | (S)-3-(4-(((R)-1-(3-(difluoromethyl)-2- fluorophenyl)ethyl)amino)quinolin-6-yl-2-d)-3- methoxy-N,N-dimethylpyrrolidine-1- carboxamide |
| 130 | 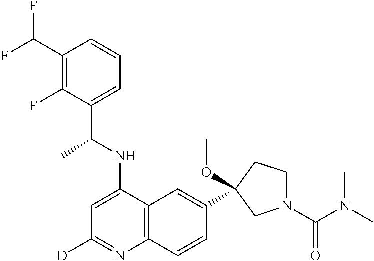 [SMILES: [2H]c1cc(N[C@H](C)c2cccc(C(F)F)c2F)c2cc([C@]3(OC)CCN(C(=O)N(C)C)C3)ccc2n1] | (R)-3-(4-(((R)-1-(3-(difluoromethyl)-2- fluorophenyl)ethyl)amino)quinolin-6-yl-2-d)-3- methoxy-N,N-dimethylpyrrolidine-1- carboxamide |

### Pharmaceutical Compositions

Pharmaceutical compositions of the present disclosure comprise at least one compound of Formula (1), or a tautomer, stereoisomer or a mixture of stereoisomers, a pharmaceutically acceptable salt, hydrate, or deuterated derivative thereof formulated together with one or more pharmaceutically acceptable carriers. These formulations include those suitable for oral, rectal, topical, buccal, and parenteral (e.g., subcutaneous, intramuscular, intradermal, or intravenous) administration. The most suitable form of administration in any given case will depend on the degree and severity of the condition being treated and on the nature of the particular compound being used.

Formulations suitable for oral administration may be presented in discrete units, such as capsules, cachets, lozenges, or tablets, each containing a predetermined amount of a compound of the present disclosure as powder or granules; as a solution or a suspension in an aqueous or non-aqueous liquid; or as an oil-in-water or water-in-oil emulsion. As indicated, such formulations may be prepared by any suitable method of pharmacy which includes the step of bringing into association at least one compound of the present disclosure as the active compound and a carrier or excipient (which may constitute one or more accessory ingredients). The carrier must be acceptable in the sense of being compatible with the other ingredients of the formulation and must not be deleterious to the recipient. The carrier may be a solid or a liquid, or both, and may be formulated with at least one compound described herein as the active compound in a unit-dose formulation, for example, a tablet, which may contain from about 0.05% to about 95% by weight of the at least one active compound. Other pharmacologically active substances may also be present including other compounds. The formulations of the present disclosure may be prepared by any of the well-known techniques of pharmacy consisting essentially of admixing the components.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by, for example, dissolving or dispersing, at least one active compound of the present disclosure as described herein and optional pharmaceutical adjuvants in an excipient, such as, for example, water, saline, aqueous dextrose, glycerol, ethanol, and the like, to thereby form a solution or suspension. In general, suitable formulations may be prepared by uniformly and intimately admixing the at least one active compound of the present disclosure with a liquid or finely divided solid carrier, or both, and then, if necessary, shaping the product. For example, a tablet may be prepared by compressing or molding a powder or granules of at least one compound of the present disclosure, which may be optionally combined with one or more accessory ingredients. Compressed tablets may be prepared by compressing, in a suitable machine, at least one compound of the present disclosure in a free-flowing form, such as a powder or granules, which may be optionally mixed with a binder, lubricant, inert diluent and/or surface active/dispersing agent(s). Molded tablets may be made by molding, in a suitable machine, where the powdered form of at least one compound of the present disclosure is moistened with an inert liquid diluent.

Formulations suitable for buccal (sub-lingual) administration include lozenges comprising at least one compound of the present disclosure in a flavored base, usually sucrose and acacia or tragacanth, and pastilles comprising the at least one compound in an inert base such as gelatin and glycerin or sucrose and acacia.

Formulations of the present disclosure suitable for parenteral administration comprise sterile aqueous preparations of at least one compound of Formula (1), or a tautomer, stereoisomer or a mixture of stereoisomers, a pharmaceutically acceptable salt, hydrate, or deuterated derivative thereof, which are approximately isotonic with the blood of the intended recipient. These preparations are administered intravenously, although administration may also be affected by means of subcutaneous, intramuscular, or intradermal injection. Such preparations may conveniently be prepared by admixing at least one compound described herein with water and rendering the resulting solution sterile and isotonic with the blood. Injectable compositions according to the present disclosure may contain from about 0.1 to about 5% w/w of the active compound.

Formulations suitable for rectal administration are presented as unit-dose suppositories. These may be prepared by admixing at least one compound as described herein with one or more conventional solid carriers, for example, cocoa butter, and then shaping the resulting mixture.

Formulations suitable for topical application to the skin may take the form of an ointment, cream, lotion, paste, gel, spray, aerosol, or oil. Carriers and excipients which may be used include Vaseline, lanoline, polyethylene glycols, alcohols, and combinations of two or more thereof. The active compound (i.e., at least one compound of Formula (1), or a tautomer, stereoisomer or a mixture of stereoisomers, or a pharmaceutically acceptable salt, or hydrate, or deuterated derivative thereof) is generally present at a concentration of from about 0.1% to about 15% w/w of the composition, for example, from about 0.5 to about 2%.

The amount of active compound administered may be dependent on the subject being treated, the subject's weight, the manner of administration, and/or the judgment of the prescribing physician. For example, a dosing schedule may involve the daily or semi-daily administration of the encapsulated compound at a perceived dosage of about 1 μg to about 1000 mg. In another embodiment, intermittent administration, such as on a monthly or yearly basis, of a dose of the encapsulated compound may be employed. Encapsulation facilitates access to the site of action and allows the administration of the active ingredients simultaneously, in theory producing a synergistic effect. In accordance with standard dosing regimens, physicians will readily determine optimum dosages and will be able to readily modify administration to achieve such dosages.

A therapeutically effective amount of a compound or composition disclosed herein can be measured by the therapeutic effectiveness of the compound. The dosages, however, may be varied depending upon the requirements of the patient, the severity of the condition being treated, and the compound being used. In one embodiment, the therapeutically effective amount of a disclosed compound is sufficient to establish a maximal plasma concentration. Preliminary doses as, for example, determined according to animal tests, and the scaling of dosages for human administration is performed according to art-accepted practices.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

Data obtained from the cell culture assays or animal studies can be used in formulating a range of dosage for use in humans. Therapeutically effective dosages achieved in one animal model may be converted for use in another animal, including humans, using conversion factors known in the art (see, e.g., Freireich et al., Cancer Chemother. Reports 50(4):219-244 (1966) and the following Table for Equivalent Surface Area Dosage Factors).

TABLE 2
Equivalent Surface Area Dosage Factors.

To:

| | Mouse | Rat | Monkey | Dog | Human |
|---|---|---|---|---|---|
| From: | (20 g) | (150 g) | (3.5 kg) | (8 kg) | (60 kg) |
| Mouse | 1 | 1/2 | 1/4 | 1/6 | 1/12 |
| Rat | 2 | 1 | 1/2 | 1/4 | 1/7 |
| Monkey | 4 | 2 | 1 | 3/5 | 1/3 |
| Dog | 6 | 4 | 3/5 | 1 | 1/2 |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and/or gender, as well as the severity of the medical condition in the subject. The dosage may be determined by a physician and adjusted, as necessary, to suit observed effects of the treatment.

### Methods of Treatment

In some embodiments, a compound of Formula (1), or a tautomer, stereoisomer or a mixture of stereoisomers, a pharmaceutically acceptable salt, hydrate, or deuterated derivative thereof, is administered to treat cancer in a subject in need thereof. In some embodiments, the cancer is chosen from breast cancer, lung cancer, pancreatic cancer, small bowel cancer, colorectal cancer, gall bladder cancer, thyroid cancer, liver cancer, bile duct cancer, ovarian cancer, endometrial cancer, cervical cancer, bladder cancer, prostate cancer, esophageal cancer, and blood cancer. In some embodiments, the cancer is lung cancer. In some embodiments, the cancer is non-small lung cancer. In some embodiments, the cancer is pancreatic cancer. In some embodiments, the cancer is colorectal cancer. In some embodiments, the therapeutic treatment is for the treatment of KRAS G12-associated diseases and conditions.

In some embodiments, a compound of Formula (1), or a tautomer, stereoisomer or a mixture of stereoisomers, a pharmaceutically acceptable salt, hydrate, or deuterated derivative thereof is administered as a pharmaceutical composition.

In some embodiments, the disclosure provides for methods for inhibiting SOS1 activity in a cell, comprising contacting the cell in which inhibition of SOS1 activity is desired with an effective amount of a compound of Formula (1), pharmaceutically acceptable salts thereof or pharmaceutical compositions containing the compound or pharmaceutically acceptable salt thereof. In one embodiment, the contacting is in vitro. In one embodiment, the contacting is in vivo.

As used herein, the term “contacting” refers to the bringing together of indicated moieties in an in vitro system or an in vivo system. For example, “contacting” a SOS1 with a compound provided herein includes the administration of a compound provided herein to an individual or patient, such as a human, having SOS1, as well as, for example, introducing a compound provided herein into a sample containing a cellular or purified preparation containing the SOS1.

In one embodiment, a cell in which inhibition of SOS1 activity is desired is contacted with an effective amount of a compound of Formula (1) to negatively modulate the activity of SOS1. In some embodiments, a therapeutically effective amount of pharmaceutically acceptable salt or pharmaceutical compositions containing the compound of Formula (1) may be used.

By negatively modulating the activity of SOS1, the methods described herein are designed to inhibit undesired cellular proliferation resulting from enhanced SOS1 activity within the cell. The cells may be contacted in a single dose or multiple doses in accordance with a particular treatment regimen to affect the desired negative modulation of SOS1.

The concentration and route of administration to the patient will vary depending on the cancer to be treated.

In one embodiment, a compound of Formula (1), or a tautomer, stereoisomer or a mixture of stereoisomers, a pharmaceutically acceptable salt, hydrate, or deuterated derivative thereof, is administered in combination with another therapeutic agent, e.g., chemotherapy, or used in combination with other treatments, such as radiation or surgical intervention, either as an adjuvant prior to surgery or post-operatively.

In some embodiments, the subject has been previously treated with an anti-cancer agent. In some embodiments, a compound of Formula (1), or a pharmaceutically acceptable salt or solvate thereof, as defined herein, may be administered to a subject in combination with an anti-cancer agent. In some embodiments, the anti-cancer agent is BRAF inhibitor. In some embodiments, the anti-cancer agent is MEK inhibitor. In some embodiments, the anti-cancer agent is ERK inhibitor. In some embodiments, the anti-cancer agent is SHP2 inhibitor. In some embodiments, the anti-cancer agent is SOS1 inhibitor. In some embodiments, the anti-cancer agent is PI3K inhibitor. In some embodiments, the anti-cancer agent is AKT inhibitor. In some embodiments, the anti-cancer agent is PD1/PDL1 inhibitor. In some embodiments, the anti-cancer agent is NRF2 inhibitor. In some embodiments, the anti-cancer agent is AMPK activator. In some embodiments, the anti-cancer agent is WNT inhibitor. In some embodiments, the anti-cancer agent is an mTOR inhibitor. In some embodiments, the anti-cancer agent is an Insulin-like Growth Factor 1 receptor (IGF-1R) inhibitor. In some embodiments, the anti-cancer agent is an epidermal growth factor receptor (EGFR) inhibitor. In some embodiments, the EGFR inhibitor is cetuximab. In some embodiments, the EGFR inhibitor is afatinib.

Also provided herein is a compound of Formula (1), or a pharmaceutically acceptable salt or solvate thereof, or a pharmaceutical composition thereof as defined herein for use in therapy.

Also provided herein is a compound of Formula (1), or a pharmaceutically acceptable salt or solvate thereof, or a pharmaceutical composition thereof as defined herein for use in the treatment of cancer. In some embodiments, the amount of a compound of Formula I in the pharmaceutical composition is in a range of from 1 mg to 1000 mg.

Also provided herein is a compound of Formula (1), or a pharmaceutically acceptable salt or solvate thereof for use in the inhibition of SOS1.

Also provided herein is a compound of Formula (1), or a pharmaceutically acceptable salt or solvate thereof, or a pharmaceutical composition thereof as defined herein, for use in the treatment of a SOS1-associated disease or disorder.

Also provided herein is the use of a compound of Formula (1), or a pharmaceutically acceptable salt or solvate thereof, as defined herein in the manufacture of a medicament for the treatment of cancer.

Also provided herein is a use of a compound of Formula (1), or a pharmaceutically acceptable salt or solvate thereof, as defined herein in the manufacture of a medicament for the inhibition of activity of SOS1.

Also provided herein is the use of a compound of Formula (1), or a pharmaceutically acceptable salt or solvate thereof, as defined herein, in the manufacture of a medicament for the treatment of a SOS1-associated disease or disorder.

### EXAMPLES

The examples and preparations provided below further illustrate and exemplify the compounds as disclosed herein and methods of preparing such compounds. It is to be understood that the scope of the present disclosure is not limited in any way by the scope of the following examples and preparations.

The chemical entities described herein can be synthesized according to one or more illustrative schemes herein and/or techniques well known in the art. Unless specified to the contrary, the reactions described herein take place at atmospheric pressure, generally within a temperature range from about −78° C. to about 200° C. Further, except as otherwise specified, reaction times and conditions are intended to be approximate, e.g., taking place at about atmospheric pressure within a temperature range of about −78° C. to about 200° C. over a period that can be, for example, about 1 to about 24 hours; reactions left to run overnight in some embodiments can average a period of about 16 hours.

Isolation and purification of the chemical entities and intermediates described herein can be affected, if desired, by any suitable separation or purification procedure such as, for example, filtration, extraction, crystallization, column chromatography, thin-layer chromatography or thick-layer chromatography, or a combination of these procedures. See, e.g., Carey et al. Advanced Organic Chemistry, 3^{rd} Ed., 1990 New York: Plenum Press; Mundy et al., Name Reaction and Reagents in Organic Synthesis, 2^{nd} Ed., 2005 Hoboken, NJ: J. Wiley & Sons. Specific illustrations of suitable separation and isolation procedures are given by reference to the examples hereinbelow. However, other equivalent separation or isolation procedures can also be used.

In all of the methods, it is well understood that protecting groups for sensitive or reactive groups may be employed where necessary, in accordance with general principles of chemistry. Protecting groups are manipulated according to standard methods of organic synthesis (T. W. Greene and P. G. M. Wuts (1999) Protective Groups in Organic Synthesis, 3^{rd} Ed., John Wiley & Sons). These groups may be removed at a convenient stage of the compound synthesis using methods that are readily apparent to those skilled in the art.

When desired, the (R)- and (S)-isomers of the nonlimiting exemplary compounds, if present, can be resolved by methods known to those skilled in the art, for example, by formation of diastereoisomeric salts or complexes which can be separated, e.g., by crystallization; via formation of diastereoisomeric derivatives which can be separated, e.g., by crystallization, gas-liquid or liquid chromatography; selective reaction of one enantiomer with an enantiomer-specific reagent, e.g., enzymatic oxidation or reduction, followed by separation of the modified and unmodified enantiomers; or gas-liquid or liquid chromatography in a chiral environment, e.g., on a chiral support, such as silica with a bound chiral ligand or in the presence of a chiral solvent. Alternatively, a specific enantiomer can be synthesized by asymmetric synthesis using optically active reagents, substrates, catalysts or solvents, or by converting one enantiomer to the other by asymmetric transformation.

The compounds described herein can be optionally contacted with a pharmaceutically acceptable acid to form the corresponding acid addition salts. Also, the compounds described herein can be optionally contacted with a pharmaceutically acceptable base to form the corresponding basic addition salts.

In some embodiments, disclosed compounds can generally be synthesized by an appropriate combination of generally well-known synthetic methods. Techniques useful in synthesizing these chemical entities are both readily apparent and accessible to those of skill in the relevant art, based on the instant disclosure. Many of the optionally substituted starting compounds and other reactants are commercially available, e.g., from Millipore Sigma or can be readily prepared by those skilled in the art using commonly employed synthetic methodology.

The discussion below is offered to illustrate certain of the diverse methods available for use in making the disclosed compounds and is not intended to limit the scope of reactions or reaction sequences that can be used in preparing the compounds provided herein. The skilled artisan will understand that standard atom valencies apply to all compounds disclosed herein in genus or named compound for unless otherwise specified.

The following abbreviations have the definitions set forth below:
1. ACN: Acetonitrile
2. AcOH Acetic acid
3. Ac₂O Acetic anhydride
4. BINAP 2,2′-bis(diphenylphosphino)-1,1′-binaphthyl
5. Boc Tert-butyloxycarbonyl protecting group
6. DAST Diethylaminosulfur trifluoride
7. DCE: Dichloroethane
8. DCM: Dichloromethane
9. DIEA: Diisopropylethylamine
10. DMA: N,N-dimethylacetamide
11. DMEDA 1,2-Dimethylethylenediamine
12. DMF Dimethylformamide
13. DMSO Dimethyl sulfoxide
14. EA/EtOAc: Ethyl acetate
15. EDCI 1-Ethyl-3-(3-dimethylaminopropyl)carbodiimide
16. FA Fluoroacetic acid
17. HOBt Hydroxybenzotriazole
18. HPLC: High pressure liquid chromatography
19. iPrMgBr Isopropylmagnesium bromide solution
20. LC/MS: Liquid chromatography/Mass spectroscopy
21. MeOH Methanol
22. n-BuLi n-Butyllithium
23. NMR: Nuclear magnetic resonance
24. PE: Polyethylene
25. Pd₂(dba)₃: Tris(dibenzylideneacetone)dipalladium (0)
26. Pd(PPH3)4 Palladium-tetrakis(triphenylphosphine)
27. RuPhos: 2-Dicyclohexylphosphino-2′,6′-diisopropoxybiphenyl
28. RuPhos-Pd-G3: (2-Dicyclohexylphosphino-2′,6′-diisopropoxy-1,1′-biphenyl)[2-(2′-amino-1,1′-biphenyl)]palladium(II) methanesulfonate
29. SFC Supercritical fluid chromatography
30. TEA: Triethylamine
31. t-BuOH tert-butyl alcohol
32. t-BuONa Sodium tert-butoxide
33. THF Tetrahydrofuan
34. TFA Trifluoroacetic acid
35. TLC: Thin layer chromatography
36. TR-FRET: Time-resolved fluorescence energy transfer
37. XPhos: 2-Dicyclohexylphosphino-2′,4′,6′-triisopropylbiphenyl
38. XPhos-Pd-G3: (2-Dicyclohexylphosphino-2′,4′,6′-triisopropyl-1,1′-biphenyl)[2-(2′-amino-1,1′-biphenyl)]palladium(II) methanesulfonate

### General Synthetic Schemes

HPLC spectra for all compounds were acquired using an Agilent 1200 Series system with DAD detector. Chromatography was performed on a 2.1×150 mm Zorbax 300SB—C18 5 μm column with water containing 0.1% formic acid as solvent A and acetonitrile containing 0.1% formic acid as solvent B at a flow rate of 0.4 mL/min. The gradient program was as follows: 1% B (0-1 min), 1-99% B (1-4 min), and 99% B (4-8 min). High-resolution mass spectra (HRMS) data were acquired in positive ion mode using an Agilent G1969A API-TOF with an electrospray ionization (ESI) source. Nuclear Magnetic Resonance (NMR) spectra were acquired on a Bruker spectrometer with 600 MHz or 400 MHz for proton (¹H NMR) and 150 MHz for carbon (¹³C NMR); chemical shifts are reported in (δ). Preparative HPLC was performed on Agilent Prep 1200 series with UV detector set to 254 nm and 220 nm. Samples were injected onto a Phenomenex Luna 75×30 mm, 5 μm, C₁₈ column at room temperature. The flow rate was 40 mL/min. A linear gradient was used with 10% (or 50%) of MeOH (A) in H₂O (with 0.1% TFA) (B) to 100% of MeOH (A). HPLC was used to establish the purity of target compounds. All final compounds were determined to be >95% purity when analyzed according to the HPLC methods described above.

Compounds of Formula (1) (see compounds in Table 1) can be prepared according to the following schemes. The following schemes represent the general methods used in preparing these compounds. However, the synthesis of these compounds is not limited to these representative methods, as they can also be prepared by various other methods those skilled in the art of synthetic chemistry, for example, in a stepwise or modular fashion.

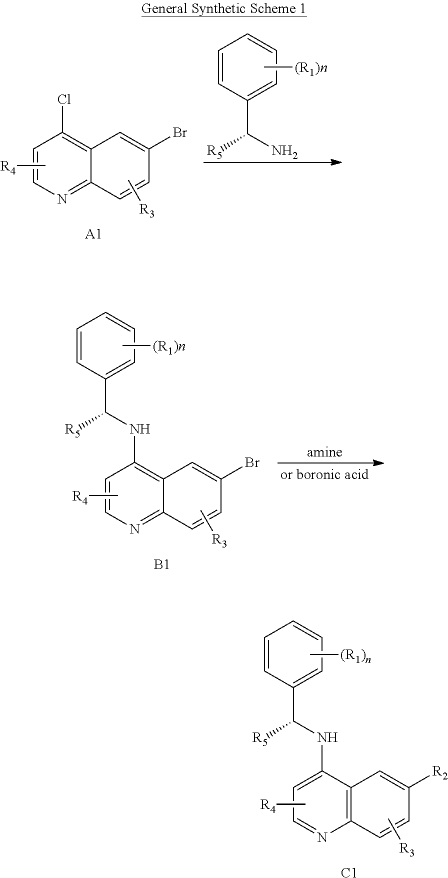

For General Synthetic Scheme 1, Compound C1 is an example of Formula (I). In this General Synthetic Scheme 1, 6-bromo-4-chloroquinoline derivative A1 is reacted with a substituted benzylamine intermediate, this reaction could for example be a nucleophilic substitution or a metal catalyzed reaction, to yield Compound B1. Compound B1 can then undergo a metal catalyzed reaction with a coupling partner, such as an amine or a boronic acid derivative to form title compound C1.

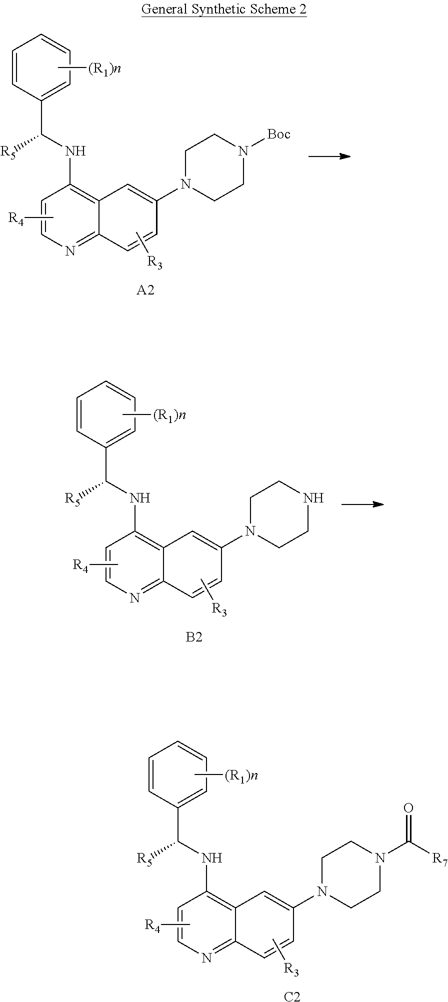

For General synthetic Scheme 2, Compound C2 is an example of Formula (I). In this General Synthetic Scheme 2, intermediate A2 can be prepared according to Scheme 1, the Boc group of A2 can be deprotected under acidic conditions such as TFA or HCl to yield intermediate B2, which can react with acyl chlorides, anhydrides, or carboxylic acids under standard amidation conditions to form compound C2.

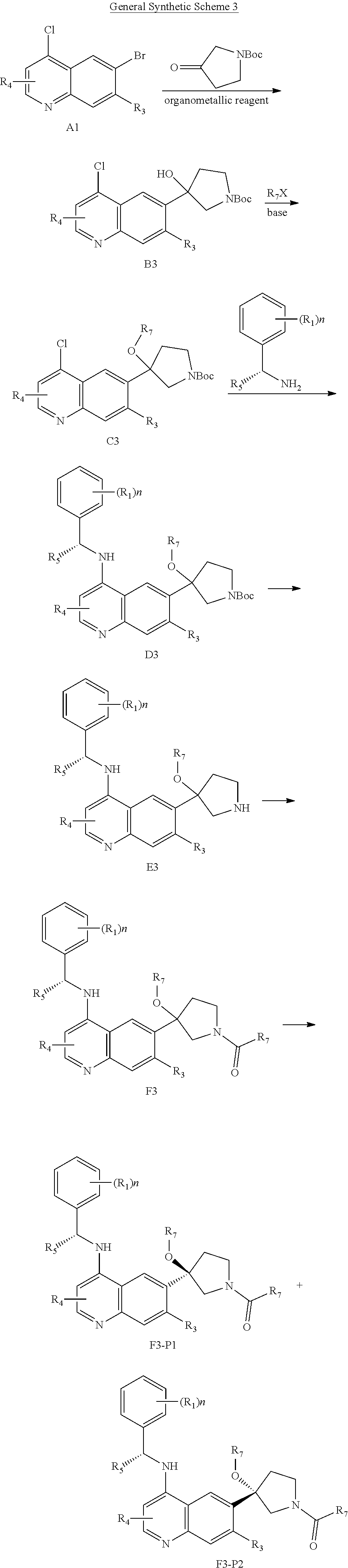

For General Synthetic Scheme 3, compounds F3, F3-P1 and F3-P2 are examples of Formula (I). In this general scheme, 6-bromo-4-chloroquinoline derivative A1 can undergo halogen metal exchange with an organometallic reagent such as a Grignard reagent or alkyl lithium reagent, and then react with Boc protected amino ketones such as pyrrolidinone, piperidinone, or homopiperidinone to form compound B3. The hydroxyl group of B3 can be alkylated with an alkyl halide in the presence of a base such as NaH to provide compound C3. By following the general synthetic schemes 1 and 2, compounds F3, F3-P1 and F3-P2 can be prepared from compound C3 with chiral separation to separate the stereoisomers.

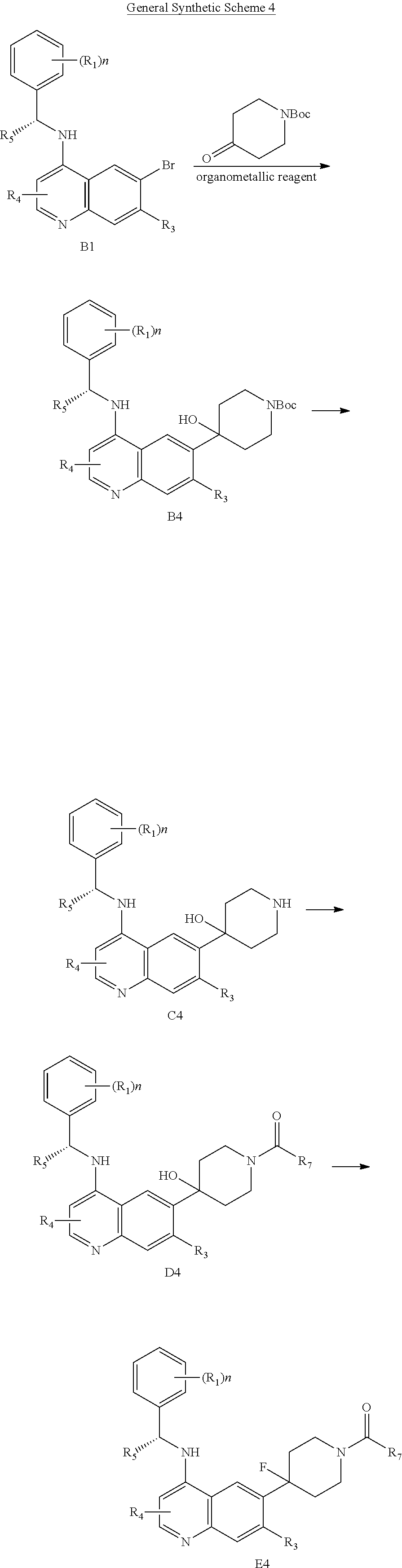

For General Synthetic Scheme 4, compounds E4 are examples of compounds of Formula (I). In this general scheme, intermediate B1 prepared according to general scheme 1 can undergo the same synthetic reactions as described in general scheme 3 to provide compound D4. The hydroxyl group of D4 can react with a fluorination reagent such as DAST to provide the fluoro compound E4. In step 1, Boc 4-piperidinone can be replaced by other Boc protected amino ketones such as but not limited to Boc pyrrolidinone and Boc-3-piperidinone.

### PREPARATION OF EXAMPLE COMPOUNDS

### Example 1: Synthesis of (R)-2-methyl-N-(1-(2-methyl-3-(trifluoromethyl)phenyl)ethyl)-6-(piperazin-1-yl)quinolin-4-amine (Compound 1)

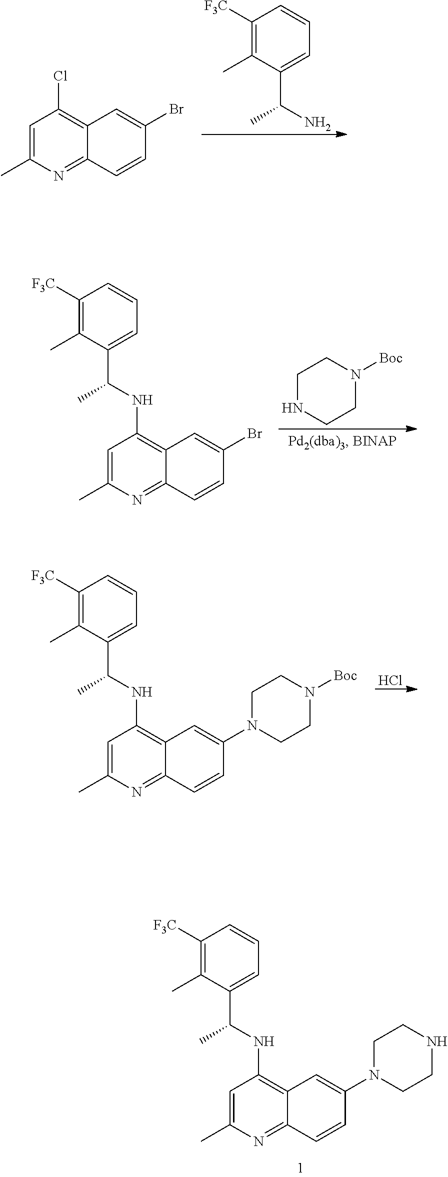

### Step 1: Preparation of (R)-6-bromo-2-methyl-N-(1-(2-methyl-3-(trifluoromethyl)phenyl)ethyl) quinolin-4-amine

To a solution of 6-bromo-4-chloro-2-methylquinoline (250 mg, 0.97 mmol) in DMSO (6 mL) was added (R)-1-(2-methyl-3-(trifluoromethyl)phenyl)ethan-1-amine hydrochloride (352 mg, 1.45 mmol) and K₂CO₃ (405 mg, 2.93 mmol). The reaction was stirred at 140° C. for 5 hours. The mixture was diluted with H₂O (15 mL) and extracted with EtOAc (15 mL×2). The combined organic layers were washed with brine and evaporated in vacuum. The residue was purified by flash chromatography (0˜ 10% MeOH in DCM) to give the product (200 mg, 48.5%) as a yellow solid. LC/MS: 423.0 [M+1]⁺.

### Step 2: Preparation of tert-butyl (R)-4-(2-methyl-4-((1-(2-methyl-3-(trifluoromethyl)phenyl)ethyl) amino)quinolin-6-yl)piperazine-1-carboxylate

To a solution of (R)-6-bromo-2-methyl-N-(1-(2-methyl-3-(trifluoromethyl)phenyl)ethyl)quinolin-4-amine (200 mg, 0.47 mmol) in THF (10 mL) was added tert-butyl piperazine-1-carboxylate (177 mg, 0.94 mmol), t-BuONa (136 mg, 1.41 mmol), BINAP (59 mg, 0.09 mmol), and Pd₂(dba)₃ (86 mg, 0.09 mmol) under argon. The mixture was stirred at 70° C. for 16 hours. The mixture was cooled to room temperature, diluted with H₂O (15 mL) and extracted with DCM (15 mL×2). The organic phase was dried over sodium sulfate and evaporated under vacuum. The residue was purified by flash chromatography (0˜ 7% MeOH in DCM) to give the desired product (130 mg, 49.3%) as a yellow solid. LC/MS: 529.2 [M+H]⁺.

### Step 3: Preparation of (R)-2-methyl-N-(1-(2-methyl-3-(trifluoromethyl)phenyl)ethyl)-6-(piperazin-1-yl)quinolin-4-amine (Compound 1)

To a solution of tert-butyl (R)-4-(2-methyl-4-((1-(2-methyl-3-(trifluoromethyl)phenyl)ethyl) amino)quinolin-6-yl) piperazine-1-carboxylate (100 mg, 0.19 mmol) in DCM (2 mL) was added HCl/dioxane (4 N, 2 mL). The reaction mixture was stirred at room temperature for 1 hour. The reaction mixture was evaporated to give a crude product which was purified by Prep-HPLC (10˜ 40% ACN in H₂O: 0.1% FA) to give the desired product (70 mg, Formic acid salt, 86.3%) as a yellow solid. LC/MS: 429.2 [M+H]⁺. 1 H NMR (400 MHz, DMSO-d6) δ 8.27 (s, 2H), 7.69 (d, J=7.8 Hz, 1H), 7.60 (dd, J=13.5, 8.4 Hz, 3H), 7.50-7.43 (m, 2H), 7.34 (t, J=7.8 Hz, 1H), 5.92 (s, 1H), 5.14-5.05 (m, 1H), 3.42-3.32 (m, 4H), 3.17-3.06 (m, 4H), 2.57 (s, 3H), 2.29 (s, 3H), 1.61 (d, J=6.7 Hz, 3H).

### Example 2: Synthesis of (R)-4-((1-(2-methyl-3-(trifluoromethyl)phenyl)ethyl)amino)-6-(piperazin-1-yl)quinoline-3-carbonitrile (Compound 2)

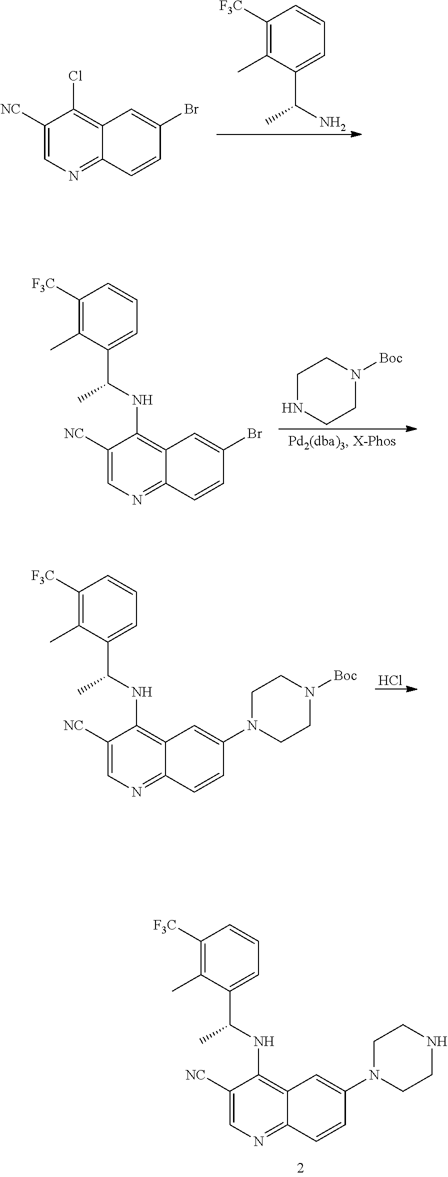

### Step 1: Preparation of (R)-6-bromo-4-((1-(2-methyl-3-(trifluoromethyl)phenyl)ethyl)amino) quinoline-3-carbonitrile

A mixture of 6-bromo-4-chloroquinoline-3-carbonitrile (900.0 mg, 3.36 mmol), (R)-1-(2-methyl-3-(trifluoromethyl)phenyl)ethan-1-amine hydrochloride salt (971.7 mg 4.04 mmol) and K₂CO₃ (1.39 g, 10.10 mmol) in DMSO (5 mL) was stirred under argon at 140° C. overnight. The reaction was quenched by adding brine (20 mL) then extracted with EA (50 mL×3). The organic phase was washed with brine and dried over Na₂SO₄. The solvent was removed in vacuum and the residue was purified by flash with PE:EA=3:1 to afford the desired product (500.0 mg, 27.3% yield) as a yellow solid. LC/MS: 433.9 [M+H]⁺.

### Step 2: Preparation of tert-butyl (R)-4-(3-cyano-4-((1-(2-methyl-3-(trifluoromethyl)phenyl)ethyl) amino)quinolin-6-yl)piperazine-1-carboxylate

To a solution (R)-6-bromo-4-((1-(2-methyl-3-(trifluoromethyl)phenyl)ethyl)amino)quinoline-3-carbonitrile (250.0 mg, 0.46 mmol) and tert-butyl piperazine-1-carboxylate (172.1 mg, 0.92 mmol) in t-BuOH (7 mL) was added X-PHOS (43.9 mg, 0.092 mmol), Pd₂(dba)₃ (84.2 mg, 0.092 mmol), and K₂CO₃ (190.5 mg, 1.38 mmol). The reaction mixture was stirred under argon at 85° C. overnight. The reaction was quenched by adding water (20 mL) then extracted with EA (50 mL×3). The organic phase was washed with brine and dried over Na₂SO₄. The solvent was removed in vacuum and the residue was purified by flash with PE:EA=1:1 to afford the desired product (110.0 mg, 39.8% yield) as a yellow solid. LC/MS: 540.3 [M+H]⁺.

### Step 3: Preparation (R)-4-((1-(2-methyl-3-(trifluoromethyl)phenyl)ethyl)amino)-6-(piperazin-1-yl)quinoline-3-carbonitrile (Compound 2)

To a solution of tert-butyl (R)-4-(3-cyano-4-((1-(2-methyl-3-(trifluoromethyl)phenyl)ethyl)amino) quinolin-6-yl)piperazine-1-carboxylate (12 mg, 0.02 mmol) in DCM (2 mL) stirred at 25° C. was added HCl/dioxane (4 N, 2 mL). The reaction mixture was stirred at room temperature for 1 hour. The solvent was removed in vacuum to afford the desired product (7 mg, HCl salt, 65.3% yield). LC/MS: 440.1 [M+H]⁺. ¹H NMR (400 MHz, DMSO-d6) δ 9.31 (s, 2H), 8.73 (s, 1H), 8.09 (s, 1H), 7.86 (dd, J=22.5, 9.1 Hz, 2H), 7.67 (dd, J=28.6, 8.0 Hz, 2H), 7.39 (t, J=7.7 Hz, 1H), 6.03-5.92 (m, 1H), 3.75-3.65 (m, 6H), 3.35-3.28 (m, 5H), 1.77 (d, J=6.6 Hz, 3H).

### Example 3: Synthesis of 4-(((R)-1-(2-methyl-3-(trifluoromethyl)phenyl)ethyl)amino)-6-(4-(tetrahydrofuran-3-carbonyl)piperazin-1-yl)quinoline-3-carbonitrile (Compound 3)

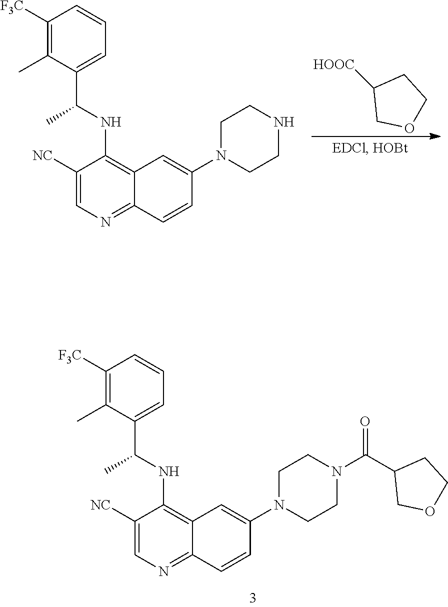 [SMILES: Cc1c([C@@H](C)Nc2c(C#N)cnc3ccc(N4CCNCC4)cc23)cccc1C(F)(F)F ; O=C(O)C1CCOC1 ; Cc1c([C@@H](C)Nc2c(C#N)cnc3ccc(N4CCN(C(=O)C5CCOC5)CC4)cc23)cccc1C(F)(F)F]

### Step 1: Preparation of 4-(((R)-1-(2-methyl-3-(trifluoromethyl)phenyl)ethyl)amino)-6-(4-(tetrahydrofuran-3-carbonyl)piperazin-1-yl)quinoline-3-carbonitrile (Compound 3)

To a solution of (R)-4-(3-cyano-4-((1-(2-methyl-3-(trifluoromethyl)phenyl)ethyl)amino)quinolin-6-yl)piperazine-1-carboxylate hydrochloride salt (100.0 mg, 0.20 mmol) and tetrahydrofuran-3-carboxylic acid (90.6 mg, 0.62 mmol) in DMF (5 mL) stirred under nitrogen at room temperature was added HOBT (42.5 mg, 0.31 mmol), EDCI (60.3 mg, 0.31 mmol), and DIEA (129.2 mg, 1.04 mmol). The reaction mixture was stirred at room temperature for 3 hours. The solvent was removed in vacuum to give a crude product. The crude product was purified by Prep-HPLC to give the title compound (60.0 mg, 52.1%). LC/MS: 538.2 [M+H]⁺. ¹H NMR (400 MHz, DMSO-d6) δ 8.21 (s, 1H), 7.80-7.72 (m, 3H), 7.72-7.55 (m, 3H), 7.35 (t, J=7.9 Hz, 1H), 5.88-5.79 (m, 1H), 3.91 (t, J=8.2 Hz, 1H), 3.80-3.63 (m, 7H), 3.51-3.35 (m, 8H), 2.20-2.05 (m, 2H), 1.68 (d, J=6.6 Hz, 3H).

### Example 4: Synthesis of (4-(2-methyl-4-(((R)-1-(2-methyl-3-(trifluoromethyl)phenyl)ethyl)amino)quinolin-6-yl)piperazin-1-yl)(tetrahydrofuran-3-yl)methanone (Compound 4)

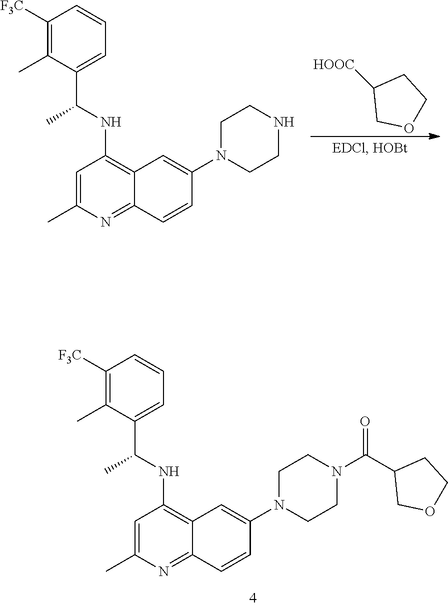 [SMILES: Cc1cc(N[C@H](C)c2cccc(C(F)(F)F)c2C)c2cc(N3CCNCC3)ccc2n1 ; O=C(O)C1CCOC1 ; Cc1cc(N[C@H](C)c2cccc(C(F)(F)F)c2C)c2cc(N3CCN(C(=O)C4CCOC4)CC3)ccc2n1]

### Step 1: Preparation of (4-(2-methyl-4-(((R)-1-(2-methyl-3-(trifluoromethyl)phenyl)ethyl)amino)quinolin-6-yl)piperazin-1-yl)(tetrahydrofuran-3-yl)methanone (Compound 4)

To a solution of (R)-2-methyl-N-(1-(2-methyl-3-(trifluoromethyl)phenyl)ethyl)-6-(piperazin-1-yl)quinolin-4-amine (110 mg, 0.24 mmol), tetrahydrofuran-3-carboxylic acid (33 mg, 0.28 mmol) and DIEA (92 mg, 0.71 mmol) in DMF (6 mL) was added HOBT (41 mg, 0.31 mmol) and EDCI (59 mg, 0.31 mmol). The reaction mixture was stirred at room temperature for 1 hour. The reaction mixture was partitioned between EA (50 mL) and water (30 mL). The organic layer was evaporated to give a crude product. The crude product was purified by Prep-HPLC (20˜ 30% ACN in H₂O: 0.1% FA) to give the desired product (17.6 mg, 14.1% yield) as a yellow solid. LC/MS: 527.2 [M+H]⁺. ¹H NMR (400 MHz, DMSO-d6) δ 8.21 (s, 1H), 7.75-7.53 (m, 4H), 7.48 (dd, J=9.2, 2.4 Hz, 1H), 7.40 (d, J=6.3 Hz, 1H), 7.34 (t, J=7.9 Hz, 1H), 5.91 (s, 1H), 5.09 (t, J=6.6 Hz, 1H), 3.91 (t, J=8.2 Hz, 1H), 3.75-3.70 (m, 7H), 3.32-3.27 (m, 4H), 2.57 (s, 3H), 2.29 (s, 3H), 2.09-2.01 (m, 2H), 1.61 (d, J=6.7 Hz, 3H).

### Example 5: Synthesis of (R)—N-(1-(2-methyl-3-(trifluoromethyl)phenyl)ethyl)-6-(piperazin-1-yl)quinolin-4-amine (Compound 5)

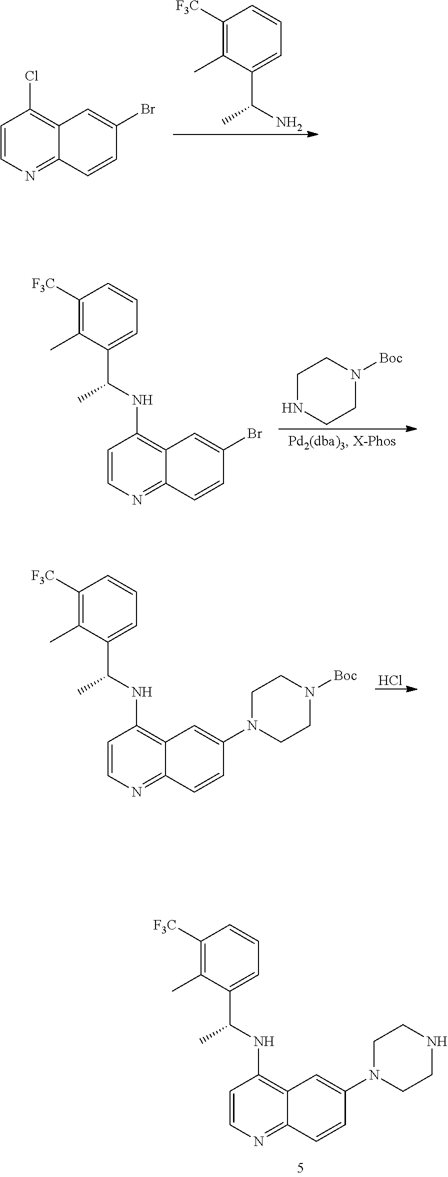

### Step 1: Preparation of (R)-6-bromo-N-(1-(2-methyl-3-(trifluoromethyl)phenyl)ethyl)quinolin-4-amine

To a solution of 6-bromo-4-chloroquinoline (300 mg, 1.24 mmol) in DMSO (6 mL) was added (R)-1-(2-methyl-3-(trifluoromethyl)phenyl)ethan-1-amine hydrochloride (387 mg, 1.61 mmol) and K₂CO₃ (513 mg, 3.71 mmol). The reaction was stirred at 140° C. for 8 hours. The mixture was diluted with H₂O (20 mL) and extracted with EtOAc (20 mL×3). The combined organic layers were washed with brine and concentrated in vacuum to give a crude product. The crude product was purified by flash chromatography (0˜ 25% EA in PE) to give the desired product (130 mg, 25.6%) as a yellow solid. LC/MS: 409.0 [M+1]⁺.

### Step 2: Preparation of tert-butyl (R)-4-(4-((1-(2-methyl-3-(trifluoromethyl)phenyl)ethyl)amino) quinolin-6-yl)piperazine-1-carboxylate

To a solution of (R)-6-bromo-N-(1-(2-methyl-3-(trifluoromethyl)phenyl)ethyl)quinolin-4-amine (65 mg, 0.16 mmol) in THF (6 mL) was added tert-butyl piperazine-1-carboxylate (59 mg, 0.32 mmol), t-BuONa (46 mg, 0.48 mmol), BINAP (20 mg, 0.03 mmol), Pd₂(dba)₃ (29 mg, 0.03 mmol) under argon. The mixture was stirred at 70° C. for 16 hours. The reaction mixture was cooled to room temperature, diluted with H₂O and extracted with DCM. The organic phase was dried over sodium sulfate and evaporated under vacuum. The residue was purified by flash chromatography (0˜ 4% MeOH in DCM) to give the desired product (80 mg, 97.7%) as a yellow solid. LC/MS: 515.2 [M+H]⁺.

### Step 3: Preparation of (R)—N-(1-(2-methyl-3-(trifluoromethyl)phenyl)ethyl)-6-(piperazin-1-yl)quinolin-4-amine (Compound 5)

To a solution of tert-butyl (R)-4-(4-((1-(2-methyl-3-(trifluoromethyl)phenyl)ethyl)amino)quinolin-6-yl)piperazine-1-carboxylate (80 mg, 0.15 mmol) in DCM (2 mL) was added HCl (4 M) in dioxane (2 mL). The reaction mixture was stirred at room temperature for 1 hour. The reaction mixture was evaporated to give the desired product. (70 mg, 100% yield). LC/MS: 415.2 [M+H]⁺. 1H NMR (400 MHz, DMSO) δ 8.23 (s, 1H), 8.07 (d, J=5.3 Hz, 1H), 7.71-7.53 (m, 4H), 7.46 (dd, J=9.3, 2.5 Hz, 1H), 7.34-7.24 (m, 2H), 5.91 (d, J=5.4 Hz, 1H), 5.04 (t, J=6.6 Hz, 1H), 3.81-3.75 (m, 5H), 3.05-3.01 (m, 4H), 2.57 (s, 3H), 1.60 (d, J=6.7 Hz, 3H).

### Example 6: Synthesis of (4-(4-(((R)-1-(2-methyl-3-(trifluoromethyl)phenyl)ethyl)amino)quinolin-6-yl)piperazin-1-yl)(tetrahydrofuran-3-yl)methanone (Compound 6)

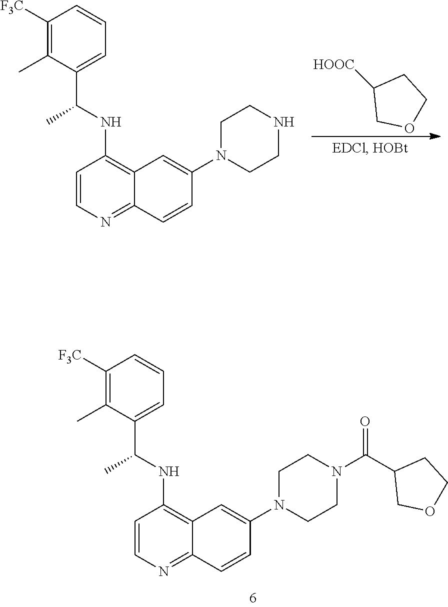 [SMILES: Cc1c([C@@H](C)Nc2ccnc3ccc(N4CCNCC4)cc23)cccc1C(F)(F)F ; O=C(O)C1CCOC1 ; Cc1c([C@@H](C)Nc2ccnc3ccc(N4CCN(C(=O)C5CCOC5)CC4)cc23)cccc1C(F)(F)F]

Compound 6 was prepared with the same procedure described for compound 3. LC/MS: 513.2 [M+H]⁺. ¹H NMR (400 MHz, DMSO-d6) δ 8.17 (s, 1H), 8.09 (d, J=5.4 Hz, 1H), 7.70-7.62 (m, 3H), 7.59-7.47 (m, 2H), 7.31 (t, J=7.8 Hz, 2H), 5.93 (d, J=5.4 Hz, 1H), 5.09-5.01 (m, 1H), 3.92 (t, J=8.2 Hz, 1H), 3.77-3.65 (m, 8H), 3.48-3.45 (m, 3H), 2.57 (s, 3H), 2.09-2.03 (m, 2H), 1.61 (d, J=6.7 Hz, 3H).

### Example 7: Synthesis of N—((R)-1-(3-cyano-4-(((R)-1-(3-(difluoromethyl)-2-fluorophenyl)ethyl)amino)quinolin-6-yl)pyrrolidin-3-yl)acetamide (Compound 7)

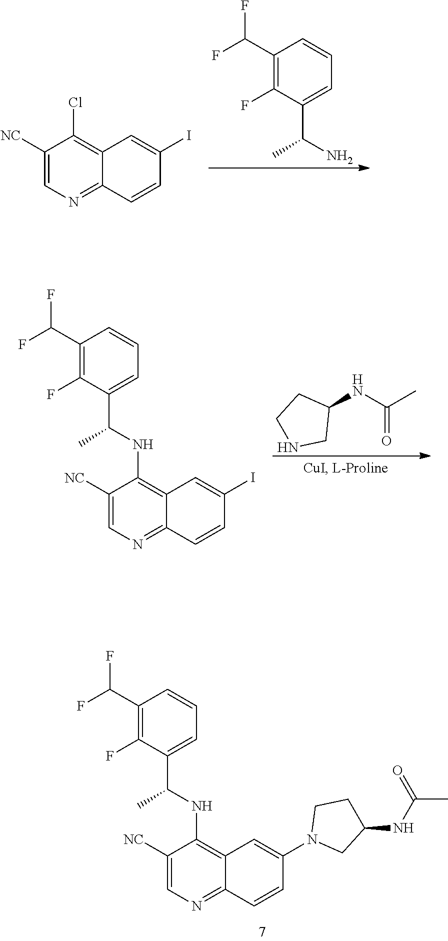

### Step 1: Preparation of (R)-4-((1-(3-(difluoromethyl)-2-fluorophenyl)ethyl)amino)-6-iodoquinoline-3-carbonitrile

The mixture of 4-chloro-6-iodoquinoline-3-carbonitrile (350 mg, 1.11 mmol), (R)-1-(3-(difluoromethyl)-2-fluorophenyl)ethan-1-amine hydrochloride (301 mg, 1.33 mmol) and DIEA (431 mg, 3.33 mmol) in dioxane (15 mL) was stirred at 100° C. for 16 hours. The mixture was concentrated in vacuum and the residue was purified by column chromatography (MeOH:DCM=0:1˜1:10) to give the product (180 mg, 34.6%) as a yellow solid. LC/MS: 467.8 [M+H]⁺.

### Step 2: Preparation of N—((R)-1-(3-cyano-4-(((R)-1-(3-(difluoromethyl)-2-fluorophenyl)ethyl)amino)quinolin-6-yl)pyrrolidin-3-yl)acetamide (Compound 7)

The mixture of (R)-4-((1-(3-(difluoromethyl)-2-fluorophenyl)ethyl)amino)-6-iodoquinoline-3-carbonitrile (80 mg, 0.17 mmol), (R)—N-(pyrrolidin-3-yl)acetamide dihydro chloride (84 mg, 0.51 mmol), Cul (13 mg, 0.068 mmol), L-Proline (16 mg, 0.14 mmol), and K₂CO₃ (142 mg, 1.03 mmol) in DMSO (6 mL) was stirred at 120° C. for 16 hours. The mixture was diluted with H₂O (20 mL) and extracted with DCM (20 mL×2). The organic phase was dried over Na₂SO₄ and concentrated in vacuum to give a crude product. The crude product was purified by Prep-HPLC to give the title compound (5 mg, 6%) as a white solid. LC/MS: 468.2 [M+H]⁺. ¹H NMR (400 MHz, DMSO-d6) δ 8.80-8.62 (m, 2H), 8.26 (d, J=6.9 Hz, 1H), 7.80 (d, J=9.2 Hz, 1H), 7.61 (t, J=7.3 Hz, 2H), 7.45-7.35 (m, 3H), 7.34-7.10 (m, 1H), 6.20-6.08 (m, 1H), 4.52-4.40 (m, 1H), 3.74-3.68 (m, 2H), 3.29-3.24 (m, 2H), 2.29-2.20 (m, 1H), 2.03-1.95 (m, 1H), 1.83 (s, 3H), 1.78 (d, J=6.6 Hz, 3H).

### Example 8: Preparation of tert-butyl ((R)-1-(4-(((R)-1-(3-(difluoromethyl)-2-fluorophenyl)ethyl)amino)quinolin-6-yl)pyrrolidin-3-yl)carbamate (Compound 8)

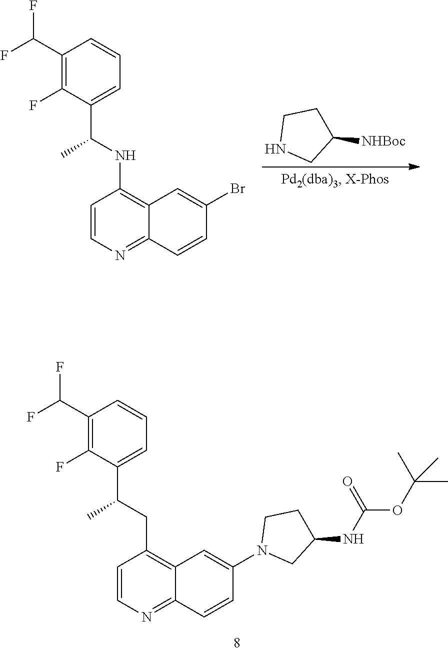 [SMILES: C[C@@H](Nc1ccnc2ccc(Br)cc12)c1cccc(C(F)F)c1F ; CC(C)(C)OC(=O)N[C@@H]1CCNC1 ; C[C@@H](Cc1ccnc2ccc(N3CC[C@@H](NC(=O)OC(C)(C)C)C3)cc12)c1cccc(C(F)F)c1F]

### Step 1: Preparation of tert-butyl ((R)-1-(4-(((R)-1-(3-(difluoromethyl)-2-fluorophenyl)ethyl)amino)quinolin-6-yl)pyrrolidin-3-yl)carbamate (Compound 8)

To a solution of (R)-6-bromo-N-(1-(3-(difluoromethyl)-2-fluorophenyl)ethyl)quinolin-4-amine (100 mg, 0.25 mmol) and tert-butyl (R)-pyrrolidin-3-ylcarbamate (139.7 mg, 0.75 mmol) in t-BuOH (10 mL) was added X-PHOS (23.9 mg, 0.05 mmol), Pd₂(dba)₃ (24.2 mg, 0.05 mmol) and K₂CO₃ (70.2 mg, 0.50 mmol). The reaction mixture was stirred under argon at 80° C. for overnight. The reaction was quenched by adding water (20 mL) and extracted with EA (50 mL×3). The organic phase was washed with brine and dried over Na₂SO₄. The solvent was removed in vacuum and the residue was purified by flash with PE:EA=1:1 to afford the desired product (45 mg, 35.9%) as a yellow solid. LC/MS: 501.2 [M+H]⁺. ¹H NMR (400 MHz, DMSO) δ 8.24 (s, 1H), 8.00 (d, J=5.2 Hz, 1H), 7.63 (d, J=9.3 Hz, 1H), 7.53 (dt, J=21.0, 7.0 Hz, 2H), 7.33-7.23 (m, 2H), 7.15-7.05 (m, 3H), 6.03 (d, J=5.4 Hz, 1H), 5.13-5.03 (m, 1H), 4.27-4.17 (m, 1H), 3.72-3.66 (m, 1H), 3.57-3.53 (m, 1H), 3.24-3.17 (m, 2H), 2.27-2.16 (m, 1H), 2.03-1.91 (m, 1H), 1.67 (d, J=6.8 Hz, 3H), 1.41 (s, 9H).

### Example 9: Preparation of 6-((R)-3-aminopyrrolidin-1-yl)-N—((R)-1-(3-(difluoromethyl)-2-fluorophenyl)ethyl)quinolin-4-amine (Compound 9)

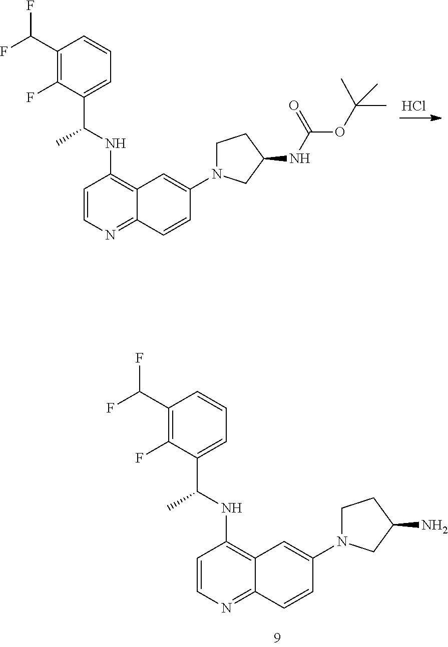 [SMILES: C[C@@H](Nc1ccnc2ccc(N3CC[C@@H](NC(=O)OC(C)(C)C)C3)cc12)c1cccc(C(F)F)c1F ; Cl ; C[C@@H](Nc1ccnc2ccc(N3CC[C@@H](N)C3)cc12)c1cccc(C(F)F)c1F]

### Step 1: Preparation of 6-((R)-3-aminopyrrolidin-1-yl)-N—((R)-1-(3-(difluoromethyl)-2-fluorophenyl)ethyl)quinolin-4-amine (Compound 9)

To a solution of tert-butyl ((R)-1-(4-(((R)-1-(3-(difluoromethyl)-2-fluorophenyl)ethyl)amino)quinolin-6-yl)pyrrolidin-3-yl)carbamate (25 mg, 0.05 mmol) in DCM (2 mL) stirred at 25° C. was added HCl/dioxane (4 N, 2 mL). The reaction mixture was stirred at room temperature for 1 hour. The solvent was removed in vacuum to afford the desired product (15 mg, HCl salt, 68.6%). LC/MS: 401.0 [M+H]⁺. ¹H NMR (400 MHz, DMSO) δ 9.02-8.83 (m, 1H), 8.41-8.11 (bs, 4H), 7.88-7.79 (m, 1H), 7.73-7.63 (m, 1H), 7.63-7.56 (m, 1H), 7.52-7.41 (m, 2H), 7.39-7.33 (m, 2H), 6.57-6.49 (m, 1H), 5.50-5.35 (m, 1H), 4.10-4.00 (m, 1H), 3.78-3.64 (m, 2H), 3.57-3.47 (m, 2H), 2.43-2.37 (m, 1H), 2.22-2.14 (m, 1H), 1.77 (d, J=6.6 Hz, 3H).

### Example 10: Preparation of (R)—N-(1-(3-(difluoromethyl)-2-fluorophenyl)ethyl)-6-(piperidin-4-yl)quinolin-4-amine (Compound 10)

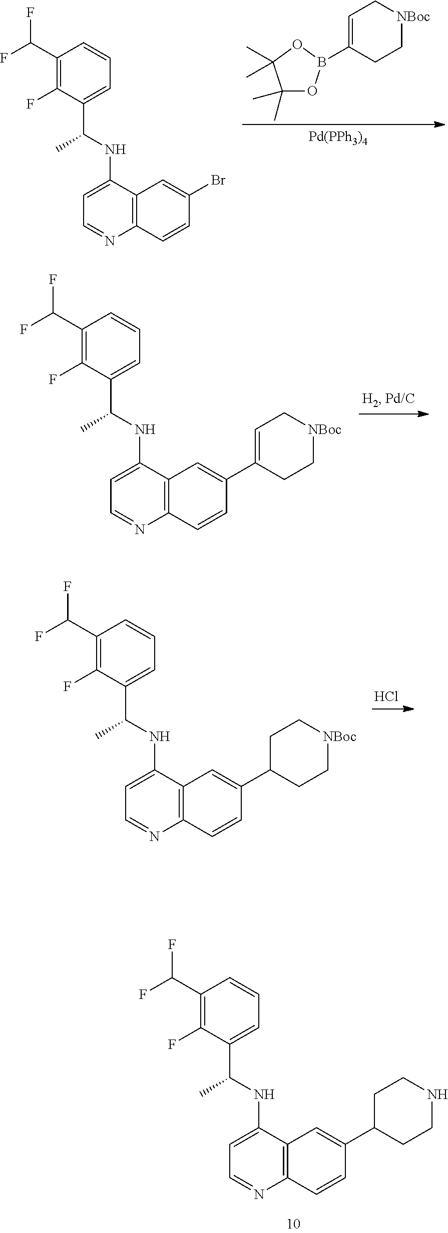

### Step 1: Preparation of tert-butyl (R)-4-(4-((1-(3-(difluoromethyl)-2-fluorophenyl)ethyl) amino)quinolin-6-yl)-3,6-dihydropyridine-1(2H)-carboxylate

A mixture of (R)-6-bromo-N-(1-(3-(difluoromethyl)-2-fluorophenyl)ethyl)quinolin-4-amine (90 mg, 0.23 mmol), tert-butyl 4-(4,4,5,5-tetramethyl-1,3,2-dioxaborolan-2-yl)-3,6-dihydropyridine-1(2H)-carboxylate (105 mg, 0.34 mmol), Pd(PPh₃)₄ (27 mg, 0.023 mmol) and K₂CO₃ (126 mg, 0.91 mmol) in dioxane/water (3:1, 8 mL) was stirred at 100° C. for 2 hours. The mixture was diluted with H₂O and extracted with DCM. The combined organic phase was concentrated in vacuum. The residue was purified by column chromatography to give the title compound (90 mg, 79.4%) as a brown solid. LC/MS: 498.2 [M+H]⁺.

### Step 2: Preparation of tert-butyl (R)-4-(4-((1-(3-(difluoromethyl)-2-fluorophenyl)ethyl) amino)quinolin-6-yl)piperidine-1-carboxylate

A mixture of tert-butyl (R)-4-(4-((1-(3-(difluoromethyl)-2-fluorophenyl)ethyl)amino) quinolin-6-yl)-3,6-dihydropyridine-1(2H)-carboxylate (90 mg, 0.18 mmol) and Pd/C (10 mg, 10%) in MeOH (2 mL) was stirred at 40° C. under H₂ atmosphere (1 atm) for 16 hours. The reaction mixture was filtered and the filtrate was evaporated to give the title compound (60 mg, 66.3%) as a brown solid. LC/MS: 499.9 [M+H]⁺.

### Step 3: Preparation of (R)—N-(1-(3-(difluoromethyl)-2-fluorophenyl)ethyl)-6-(piperidin-4-yl)quinolin-4-amine (Compound 10)

To a solution of tert-butyl (R)-4-(4-((1-(3-(difluoromethyl)-2-fluorophenyl)ethyl) amino)quinolin-6-yl)piperidine-1-carboxylate (60 mg, 0.12 mmol) in dioxane (5 mL) was added HCl/dioxane (4 N, 1 mL). The mixture was stirred at room temperature for 2 hours. The solvent was removed in vacuum and the residue was dissolved with saturated NaHCO₃ solution. The mixture was extracted with DCM, dried over Na₂SO₄ and concentrated in vacuum to give the title compound (50 mg, HCl salt, 88.2%) as a yellow solid. LC/MS: 400.0 [M+H]⁺. ¹H NMR (400 MHz, DMSO-d6) δ 14.46 (s, 1H), 9.57 (d, J=7.1 Hz, 1H), 9.28-9.03 (m, 2H), 8.74 (s, 1H), 8.51 (d, J=6.7 Hz, 1H), 8.03-7.87 (m, 2H), 7.80-7.58 (m, 2H), 7.40-7.12 (m, 2H), 6.66 (d, J=7.2 Hz, 1H), 5.51-5.40 (m, 1H), 3.46-3.40 (m, 2H), 3.14-3.00 (m, 3H), 2.14-2.00 (m, 4H), 1.78 (d, J=6.8 Hz, 3H).

### Example 11: Preparation of N—((R)-1-(4-(((R)-1-(3-(difluoromethyl)-2-fluorophenyl)ethyl)amino)quinolin-6-yl)pyrrolidin-3-yl)acetamide (Compound 11)

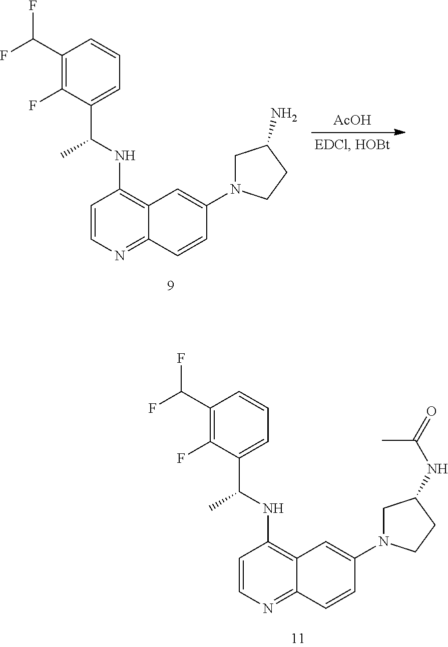 [SMILES: "AcOH" ; C[C@@H](Nc1ccnc2ccc(N3CC[C@@H](N)C3)cc12)c1cccc(C(F)F)c1F ; CC(=O)N[C@@H]1CCN(c2ccc3nccc(N[C@H](C)c4cccc(C(F)F)c4F)c3c2)C1]

### Step 1: Preparation of N—((R)-1-(4-(((R)-1-(3-(difluoromethyl)-2-fluorophenyl)ethyl)amino)quinolin-6-yl)pyrrolidin-3-yl)acetamide (Compound 11)

To a solution of tert-butyl 6-((R)-3-aminopyrrolidin-1-yl)-N—((R)-1-(3-(difluoromethyl)-2-fluoro phenyl)ethyl)quinolin-4-amine (15.0 mg, 0.037 mmol) and acetic acid (11.1 mg, 0.18 mmol) in DMF (5 mL) stirred under nitrogen at room temperature was added HOBT (7.5 mg, 0.056 mmol), EDCI (10.6 mg, 0.056 mmol), and DIEA (38.2 mg, 0.30 mmol). The reaction mixture was stirred at room temperature for 2 hours. The solvent was removed in vacuum to give a crude product. The crude product was purified by Prep-HPLC to give the title compound (10 mg, 61.0%). LC/MS: 442.9 [M+H]⁺. ¹H NMR (400 MHz, DMSO-d6) δ 8.23 (d, J=6.9 Hz, 1H), 8.18 (s, 1H), 8.03 (d, J=5.3 Hz, 1H), 7.65 (d, J=9.8 Hz, 1H), 7.59-7.49 (m, 2H), 7.30-7.24 (m, 2H), 7.17-7.12 (m, 2H), 6.10-6.05 (m, 1H), 5.14-5.08 (m, 1H), 4.49-4.43 (m, 1H), 3.71-3.66 (m, 1H), 3.58-3.54 (m, 1H), 3.25-3.20 (m, 2H), 2.28-2.23 (m, 1H), 1.99-1.92 (m, 1H), 1.84 (s, 3H), 1.68 (d, J=6.8 Hz, 3H).

### Example 12: Preparation of (R)-1-(4-(4-((1-(3-(difluoromethyl)-2-fluorophenyl)ethyl)amino)quinolin-6-yl)piperazin-1-yl)ethan-1-one (Compound 12)

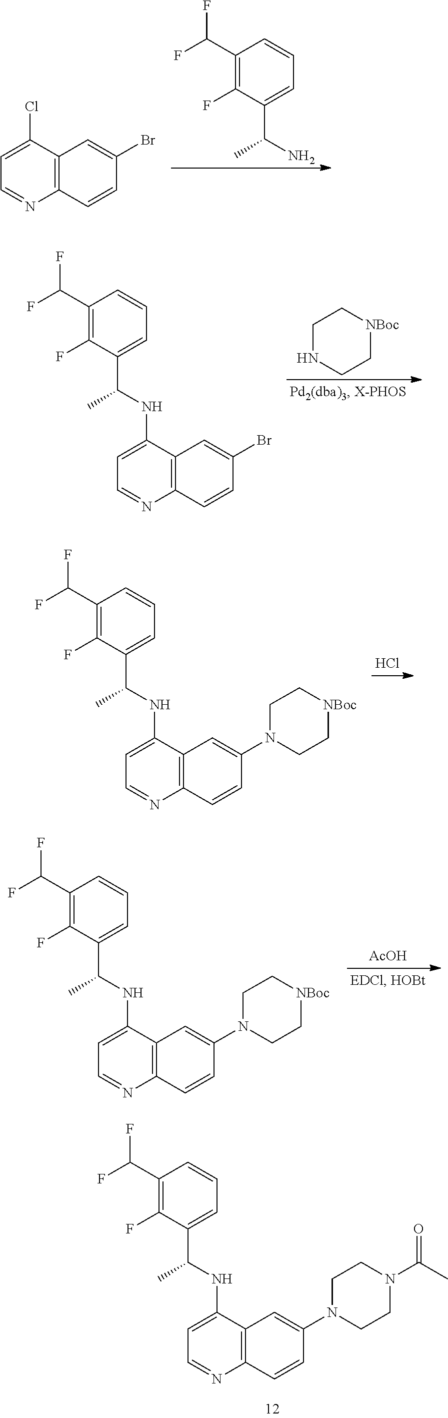

### Step 1: Preparation of (R)-6-bromo-N-(1-(3-(difluoromethyl)-2-fluorophenyl)ethyl)quinolin-4-amine

To a solution of 6-bromo-4-chloroquinoline (1.1 g, 4.536 mmol) in dry DMSO (10 mL) stirred under argon at room temperature was added (1R)-1-[3-(difluoromethyl)-2-fluorophenyl]ethanamine (1.0 g, 4.53 mmol) and K₂C₀₃ (3.1 g, 22.68 mmol). The reaction mixture was stirred at 140° C. for 2 hours. The solvent was removed in vacuum and the residue was washed with DCM (150 mL×3). The organic phase was washed with brine and dried over Na₂SO₄. After evaporation of DCM under vacuum, the crude product was purified by silica-gel column chromatography and eluted with 0-10% MeOH in DCM to give the title compound (700 mg, 39.0%) as a yellow solid. LC/MS: 395.0 [M+H]⁺.

### Step 2: Preparation of tert-butyl (R)-4-(4-((1-(3-(difluoromethyl)-2-fluorophenyl)ethyl)amino) quinolin-6-yl)piperazine-1-carboxylate

A mixture of 6-bromo-N-[(1R)-1-[3-(difluoromethyl)-2-fluorophenyl]ethyl]quinolin-4-amine (700 mg, 1.77 mmol), tert-butyl piperazine-1-carboxylate (1.65 g, 8.85 mmol), Pd₂(dba)₃ (324 mg, 0.35 mmol), X-PHOS (168 mg, 0.35 mmol), and K₂CO₃ (1.22 g, 8.85 mmol) in t-BuOH (20 mL) was stirred under nitrogen atmosphere at 85° C. for 16 hours. The mixture was concentrated in vacuum and the residue was washed with DCM (150 mL×3). The organic phase was washed with brine and dried over Na₂SO₄. After evaporation of DCM under vacuum, the crude product was purified by silica-gel column chromatography and eluted with 0-10% MeOH in DCM to give the title compound (700 mg, 78.9%) as a yellow solid. LC/MS: 501.3 [M+H]⁺.

### Step 3: Preparation of (R)—N-(1-(3-(difluoromethyl)-2-fluorophenyl)ethyl)-6-(piperazin-1-yl) quinolin-4-amine

A solution of tert-butyl 4-(4-{[(1R)-1-[3-(difluoromethyl)-2-fluorophenyl]ethyl]amino}quinoline-6-yl)piperazine-1-carboxylate (300 mg, 0.59 mmol) in HCl/dioxane (4 N, 20 mL) was stirred at room temperature for 2 hours. The solvent was removed in vacuum to give the title compound (300 mg, crude) as a yellow solid. LC/MS: 401.1 [M+H]⁺.

### Step 4: Preparation of (R)-1-(4-(4-((1-(3-(difluoromethyl)-2-fluorophenyl)ethyl)amino)quinolin-6-yl)piperazin-1-yl)ethan-1-one (Compound 12)

To a solution of N-[(1R)-1-[3-(difluoromethyl)-2-fluorophenyl]ethyl]-6-(piperazin-1-yl)quinolin-4-amine (50 mg, crude) in DMF (2 mL) stirred under argon at room temperature was added acetic acid (37.5 mg, 0.62 mmol), DIEA (80 mg, 0.62 mmol), EDCI (48 mg, 0.24 mmol), and HOBT (34 mg, 0.24 mmol). The reaction mixture was stirred at room temperature for 2 hours. The solvent was removed in vacuum and the residue was purified by Prep-HPLC to give the title compound (15 mg, 26.42%). LC/MS: 442.9 [M+H]⁺. 1H NMR (400 MHz, DMSO-d6) δ 8.22 (s, 1H), 8.11 (d, J=5.2 Hz, 1H), 7.70-7.65 (m, 1H), 7.62 (d, J=2.4 Hz, 1H), 7.58 (t, J=7.2 Hz, 1H), 7.54-7.47 (m, 2H), 7.40-7.10 (m, 3H), 6.08 (d, J=5.4 Hz, 1H), 5.07 (t, J=6.8 Hz, 1H), 3.67-3.61 (m, 4H), 3.30-3.25 (m, 4H), 2.08 (s, 3H), 1.67 (d, J=6.8 Hz, 3H).

### Example 13: Preparation of (R)-1-(4-(4-((1-(3-(difluoromethyl)-2-fluorophenyl)ethyl)amino)quinolin-6-yl)piperidin-1-yl)ethan-1-one (Compound 13)

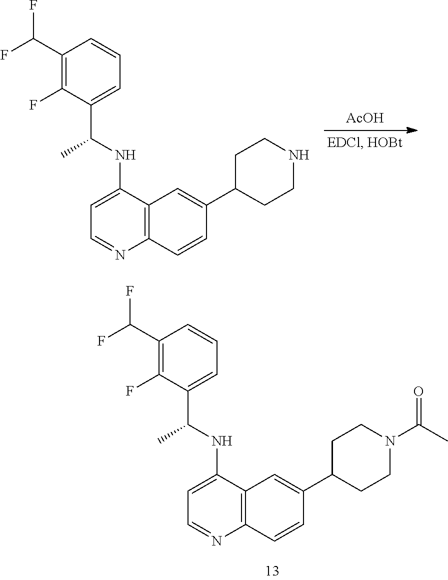

### Step 1: Preparation of (R)-1-(4-(4-((1-(3-(difluoromethyl)-2-fluorophenyl)ethyl)amino)quinolin-6-yl)piperidin-1-yl)ethan-1-one (Compound 13)

A mixture of (R)—N-(1-(3-(difluoromethyl)-2-fluorophenyl)ethyl)-6-(piperidin-4-yl) quinoline-4-amine dihydrochloride (35 mg, 0.074 mmol), acetic acid (23 mg, 0.37 mmol), DIEA (48 mg, 0.37 mmol), EDCI (29 mg, 0.15 mmol), and HOBT (20 mg, 0.15 mmol) in DMF (3 mL) was stirred at room temperature for 2 hours. The mixture was diluted with H₂O (10 mL) and extracted with DCM (10 mL×2). The combined organic phase was dried over sodium sulfate and concentrated in vacuum. The residue was purified by Prep-HPLC to give the title compound (10 mg, 30.6%) as a white solid. LC/MS: 441.9 [M+H]⁺. ¹H NMR (400 MHz, DMSO-d6) δ 8.28 (s, 1H), 8.24 (d, J=5.3 Hz, 1H), 8.17 (s, 1H), 7.75-7.69 (m, 1H), 7.62-7.44 (m, 4H), 7.40-7.13 (m, 2H), 6.13 (d, J=5.5 Hz, 1H), 5.12-5.03 (m, 1H), 4.66-4.60 (m, 1H), 4.04-3.96 (m, 1H), 3.22-3.17 (m, 1H), 3.00-2.91 (m, 1H), 2.68-2.60 (m, 1H), 2.07 (s, 3H), 1.94-1.71 (m, 4H), 1.67 (d, J=6.8 Hz, 3H).

### Example 14: Preparation of (4-(4-(((R)-1-(3-(difluoromethyl)-2-fluorophenyl)ethyl)amino)quinolin-6-yl)piperazin-1-yl)((S)-tetrahydrofuran-3-yl)methanone (Compound 14) and (4-(4-(((R)-1-(3-(difluoromethyl)-2-fluorophenyl)ethyl)amino)quinolin-6-yl)piperazin-1-yl)((R)-tetrahydrofuran-3-yl)methanone (Compound 15)

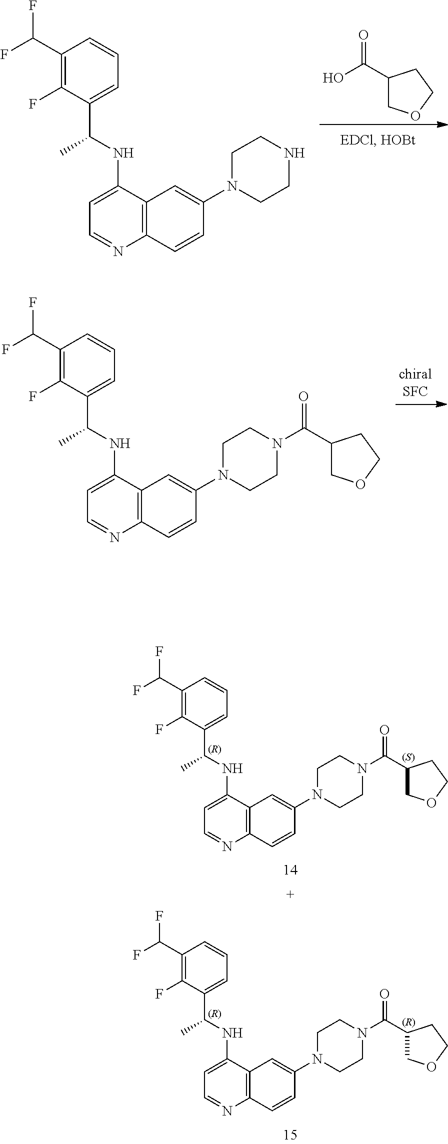

### Step 1: Preparation of (4-(4-(((R)-1-(3-(difluoromethyl)-2-fluorophenyl)ethyl)amino)quinolin-6-yl)piperazin-1-yl)(tetrahydrofuran-3-yl)methanone

To a solution of N-[(1R)-1-[3-(difluoromethyl)-2-fluorophenyl]ethyl]-6-(piperazin-1-yl)quinolin-4-amine (50 mg, 0.12 mmol) in DMF (2 mL) stirred under argon at room temperature was added oxolane-3-carboxylic acid (43 mg, 0.37 mmol), DIEA (80 mg, 0.62 mmol), EDCI (48 mg, 0.24 mmol), and HOBT (34 mg, 0.24 mmol). The reaction mixture was stirred at room temperature for 2 hours. The solvent was removed in vacuum and the residue was purified by Prep-HPLC to afford the desired compound (11 mg, 18.3%) as a yellow solid. LC/MS: 499.1 [M+H]⁺.

### Step 2: Preparation of (4-(4-(((R)-1-(3-(difluoromethyl)-2-fluorophenyl)ethyl)amino)quinolin-6-yl)piperazin-1-yl)((S)-tetrahydrofuran-3-yl)methanone (Compound 14) and (4-(4-(((R)-1-(3-(difluoromethyl)-2-fluorophenyl)ethyl)amino)quinolin-6-yl)piperazin-1-yl)((R)-tetrahydrofuran-3-yl)methanone (Compound 15)

The racemate sample was separated by chiral SFC (column: chiralpak-OJ, mobile phase: hexane/i-PrOH (DEA), gradient: 50%) to afford Compound 14 (first peak, 5.1 mg, 46%) as a yellow solid and Compound 15 (second peak, 4.8 mg, 43%) as a yellow solid.

(4-(4-(((R)-1-(3-(difluoromethyl)-2-fluorophenyl)ethyl)amino)quinolin-6-yl)piperazin-1-yl)((R)-tetrahydrofuran-3-yl)methanone: LC/MS: 499.1 [M+H]⁺. 1H NMR (400 MHz, DMSO-d6) δ 8.20 (d, J=5.6 Hz, 1H), 8.00 (bs, 1H), 7.76 (d, J=9.2 Hz, 2H), 7.70-7.59 (m, 2H), 7.55 (t, J=8.2 Hz, 1H), 7.40-7.10 (m, 1H), 6.26 (d, J=6.0 Hz, 1H), 5.21 (t, J=6.8 Hz, 1H), 3.92 (t, J=8.2 Hz, 1H), 3.77-3.70 (m, 6H), 3.50-3.41 (m, 2H), 3.39-3.34 (m, 4H), 2.10-2.02 (m, 2H), 1.72 (d, J=6.8 Hz, 3H).

(4-(4-(((R)-1-(3-(difluoromethyl)-2-fluorophenyl)ethyl)amino)quinolin-6-yl)piperazin-1-yl)((S)-tetrahydrofuran-3-yl)methanone: LC/MS: 499.1 [M+H]⁺. 1H NMR (400 MHz, DMSO-d6) δ 8.20 (d, J=5.6 Hz, 1H), 8.00 (bs, 1H), 7.76 (d, J=9.2 Hz, 2H), 7.70-7.59 (m, 2H), 7.55 (t, J=8.2 Hz, 1H), 7.40-7.10 (m, 1H), 6.26 (d, J=6.0 Hz, 1H), 5.21 (t, J=6.8 Hz, 1H), 3.92 (t, J=8.2 Hz, 1H), 3.77-3.70 (m, 6H), 3.50-3.41 (m, 2H), 3.39-3.34 (m, 4H), 2.10-2.02 (m, 2H), 1.72 (d, J=6.8 Hz, 3H).

### Example 15: Preparation of (R)-(4-(4-((1-(3-(difluoromethyl)-2-fluorophenyl)ethyl)amino)quinolin-6-yl)piperazin-1-yl)(oxetan-3-yl)methanone (Compound 16)

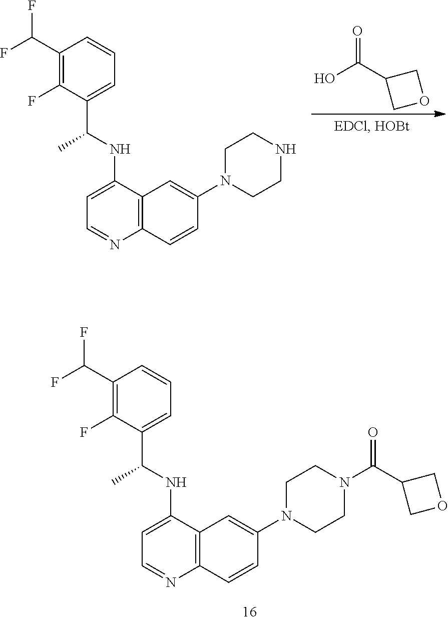 [SMILES: C[C@@H](Nc1ccnc2ccc(N3CCNCC3)cc12)c1cccc(C(F)F)c1F ; O=C(O)C1COC1 ; C[C@@H](Nc1ccnc2ccc(N3CCN(C(=O)C4COC4)CC3)cc12)c1cccc(C(F)F)c1F]

### Step 1: Preparation of (R)-(4-(4-((1-(3-(difluoromethyl)-2-fluorophenyl)ethyl)amino)quinolin-6-yl)piperazin-1-yl)(oxetan-3-yl)methanone (Compound 16)

To a solution of N-[(1R)-1-[3-(difluoromethyl)-2-fluorophenyl]ethyl]-6-(piperazin-1-yl)quinolin-4-amine (50 mg, 0.125 mmol) in DMF (2 mL) stirred under argon at room temperature was added oxetane-3-carboxylic acid (38 mg, 0.374 mmol), DIEA (80 mg, 0.624 mmol), EDCI (48 mg, 0.249 mmol), and HOBT (34 mg, 0.249 mmol). The reaction mixture was stirred at room temperature for 2 hours. The solvent was removed in vacuum and the residue was purified by Prep-HPLC to afford the desired compound (7 mg, 97.7% purity, 11.2% yield) as a yellow solid. LC/MS: 485.2 [M+H]⁺. ¹H NMR (400 MHz, DMSO-d6) δ 8.20 (s, 1H), 8.12 (d, J=5.2 Hz, 1H), 7.67 (d, J=9.2 Hz, 1H), 7.62 (d, J=2.3 Hz, 1H), 7.58 (t, J=7.3 Hz, 1H), 7.54-7.46 (m, 2H), 7.40-7.12 (m, 3H), 6.09 (d, J=5.5 Hz, 1H), 5.12-5.04 (m, 1H), 4.80-4.65 (m, 4H), 4.29-4.15 (m, 1H), 3.72 (t, J=5.2 Hz, 2H), 3.45-3.35 (m, 2H), 3.35-3.20 (m, 4H), 1.67 (d, J=6.8 Hz, 3H).

### Example 16: Preparation of (R)-1-(4-(4-((1-(3-(difluoromethyl)-2-fluorophenyl)ethyl)amino)quinolin-6-yl)piperazin-1-yl)-2-methylpropan-1-one (Compound 17)

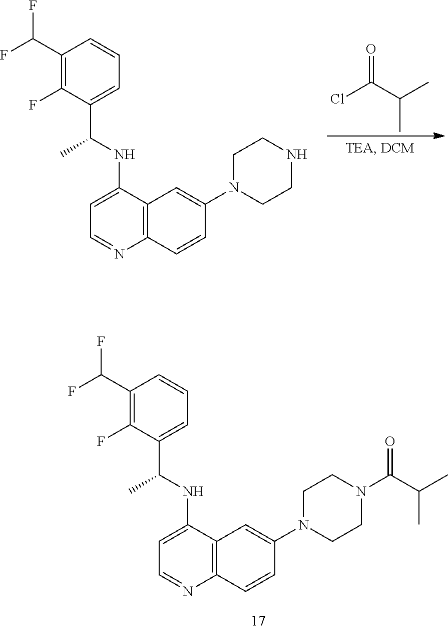

### Step 1: Preparation of (R)-1-(4-(4-((1-(3-(difluoromethyl)-2-fluorophenyl)ethyl)amino)quinolin-6-yl)piperazin-1-yl)-2-methylpropan-1-one (Compound 17)

To a solution of N-[(1R)-1-[3-(difluoromethyl)-2-fluorophenyl]ethyl]-6-(piperazin-1-yl)quinolin-4-amine (50 mg, 0.124 mmol) in DCM (5 mL) stirred under argon at room temperature was added TEA (38 mg, 0.374 mmol) and 2-methylpropanoyl chloride (13.3 mg, 0.124 mmol). The reaction mixture was stirred at room temperature for 2 hours. The solvent was removed in vacuum and the residue was purified by Prep-HPLC to afford the desired compound (12.5 mg, 20.6% yield) as a yellow solid. LC/MS:471.2 [M+H]⁺. ¹H NMR (400 MHz, DMSO-d6) δ 8.24 (s, 1H), 8.12 (d, J=5.2 Hz, 1H), 7.68 (d, J=9.2 Hz, 1H), 7.63 (d, J=2.4 Hz, 1H), 7.58 (t, J=7.2 Hz, 1H), 7.55-7.48 (m, 2H), 7.40-7.12 (m, 3H), 6.08 (d, J=5.2 Hz, 1H), 5.11-5.04 (m, 1H), 3.75-3.71 (m, 4H), 3.37-3.25 (m, 4H), 3.01-2.94 (m, 1H), 1.67 (d, J=6.8 Hz, 3H), 1.05 (d, J=6.8 Hz, 6H).

### Example 17: Preparation of 6-((S)-3-aminopyrrolidin-1-yl)-N—((R)-1-(3-(difluoromethyl)-2-fluorophenyl)ethyl)quinolin-4-amine (Compound 18)

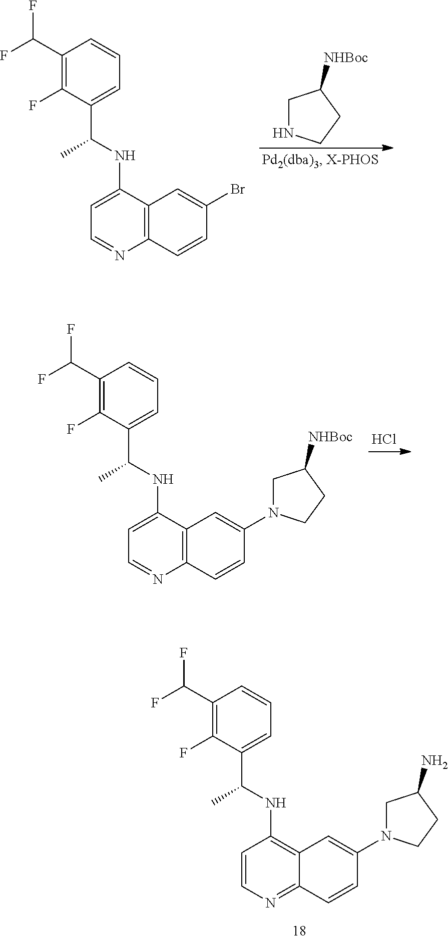 [SMILES: C[C@@H](Nc1ccnc2ccc(Br)cc12)c1cccc(C(F)F)c1F ; CC(C)(C)OC(=O)N[C@H]1CCNC1 ; C[C@@H](Nc1ccnc2ccc(N3CC[C@H](NC(=O)OC(C)(C)C)C3)cc12)c1cccc(C(F)F)c1F ; Cl ; C[C@@H](Nc1ccnc2ccc(N3CC[C@H](N)C3)cc12)c1cccc(C(F)F)c1F]

### Step 1: Preparation of tert-butyl ((S)-1-(4-(((R)-1-(3-(difluoromethyl)-2-fluorophenyl)ethyl)amino)quinolin-6-yl)pyrrolidin-3-yl)carbamate

To a solution of (R)-6-bromo-N-(1-(3-(difluoromethyl)-2-fluorophenyl)ethyl)quinolin-4-amine (45 mg, 0.11 mmol) in t-BuOH (5 mL) was added tert-butyl (S)-pyrrolidin-3-ylcarbamate (106 mg, 0.56 mmol), K₂C₀₃ (79 mg, 0.56 mmol), X-Phos (11 mg, 0.02 mmol), and Pd₂(dba)₃ (21 mg, 0.02 mmol). The reaction was stirred under nitrogen at 85° C. for 16 hours. The mixture was diluted with water (10 mL) and extracted with DCM (10 mL×2). The organic phase was washed with brine and dried over Na₂SO₄. The solvent was removed in vacuum and the residue was purified via silica gel column chromatography (PE:EA=1:1) to afford the desired compound (40 mg, 66.6%). LC/MS: 501.2[M+H]⁺.

### Step 2: Preparation of 6-((S)-3-aminopyrrolidin-1-yl)-N—((R)-1-(3-(difluoromethyl)-2-fluorophenyl)ethyl)quinolin-4-amine (Compound 18)

A solution of 6-((S)-3-aminopyrrolidin-1-yl)-N—((R)-1-(3-(difluoromethyl)-2-fluorophenyl)ethyl)quinolin-4-amine (35 mg, 0.06 mmol) in HCl/dioxane (4 N, 5 mL) and DCM (5 mL) was stirred at room temperature for 1 hour. The solvent was removed in vacuum and the residue was purified by Prep-HPLC (Xbridge-C18: 2.1×50 mm, 3.5 μm; ACN-H₂O (0.05% TFA): 0-60%) to afford the desired compound (10 mg, 33.9%). LC/MS: 401.1 [M+H]⁺. ¹H NMR (400 MHz, DMSO-d6) δ 8.25 (d, J=5.2 Hz, 1H), 8.03 (d, J=5.2 Hz, 1H), 7.66 (d, J=9.0 Hz, 1H), 7.59-7.48 (m, 2H), 7.27 (d, J=9.2 Hz, 1H), 7.22-7.11 (m, 3H), 6.06 (d, J=5.2 Hz, 1H), 5.13-5.06 (m, 1H), 3.95-3.85 (m, 1H), 3.70-3.56 (m, 3H), 3.54-3.31 (m, 3H), 2.35-2.28 (m, 1H), 2.07-1.99 (m, 1H), 1.68 (d, J=6.8 Hz, 3H).

### Example 18: Preparation of (R)-1-(4-(4-((1-(3-(difluoromethyl)-2-fluorophenyl)ethyl)amino)quinolin-6-yl)-4-hydroxypiperidin-1-yl)ethan-1-one (Compound 40)

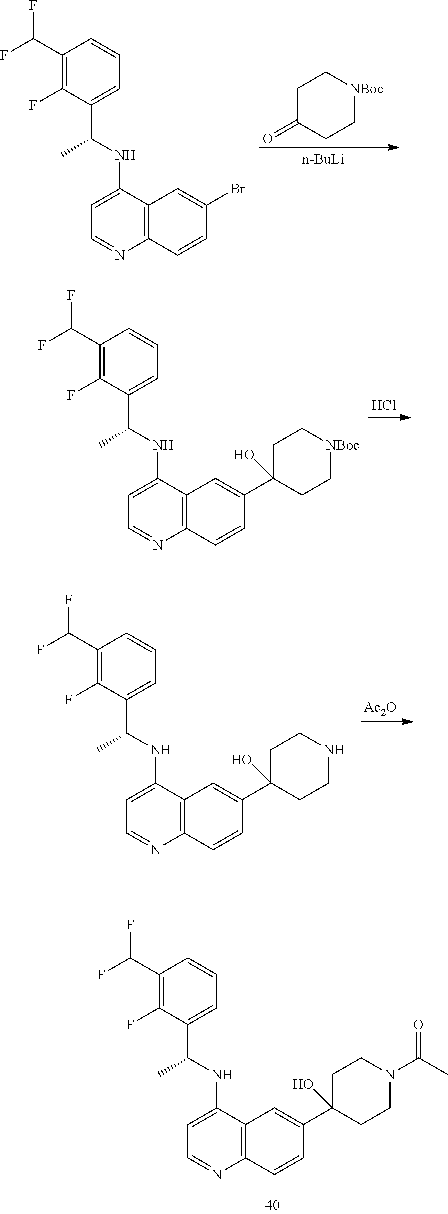

### Step 1: Preparation of tert-butyl (R)-4-(4-((1-(3-(difluoromethyl)-2-fluorophenyl)ethyl)amino)quinolin-6-yl)-4-hydroxypiperidine-1-carboxylate

To a solution of (R)-6-bromo-N-(1-(3-(difluoromethyl)-2-fluorophenyl)ethyl)quinolin-4-amine (400 mg, 1.01 mmol) in THF (2 mL) and diethyl ether (2 mL) stirred under argon at −78° C. was added n-BuLi (0.8 mL, 2.02 mmol). The mixture was stirred at −40° C. for 1 hour. A solution of tert-butyl 4-oxopiperidine-1-carboxylate (201 mg, 1.01 mmol) in THF (1 mL) was added at −40° C. and the reaction mixture was stirred at room temperature for additional 1 hour. The reaction was quenched with water and extracted with EA. The organic phase was washed with brine, dried over Na₂SO₄, and concentrated in vacuum. The residue was purified by silica gel chromatography with 0˜10% MeOH in DCM to afford the desired compound (80 mg, 15.3%) as a brown oil. LC/MS: 516.1 [M+H]⁺.

### Step 2: Preparation of (R)-4-(4-((1-(3-(difluoromethyl)-2-fluorophenyl)ethyl)amino)quinolin-6-yl)piperidin-4-ol

A solution of tert-butyl (R)-4-(4-((1-(3-(difluoromethyl)-2-fluorophenyl)ethyl)amino)quinolin-6-yl)-4-hydroxypiperidine-1-carboxylate (80 mg, 0.15 mmol) in HCl/dioxane (4 N, 5 mL) was stirred at 25° C. for 2 hours. The solvent was removed in vacuum. The residue was dissolved with EA and basified with saturated sodium bicarbonate. The organic phase was concentrated in vacuum to afford the desired compound (60 mg, 96.2%) as a yellow solid. LC/MS: 416.0 [M+H]⁺.

### Step 3: Preparation of (R)-1-(4-(4-((1-(3-(difluoromethyl)-2-fluorophenyl)ethyl)amino)quinolin-6-yl)-4-hydroxypiperidin-1-yl)ethan-1-one (Compound 40)

To a solution of (R)-4-(4-((1-(3-(difluoromethyl)-2-fluorophenyl)ethyl)amino)quinolin-6-yl) piperidin-4-ol (45 mg, 0.10 mmol) in DCM (5 mL) was added Acetic anhydride (11 mg, 0.10 mmol) and TEA (26 mg, 0.32 mmol). The reaction was stirred at room temperature for 0.5 hours. The solvent was removed in vacuum and the residue was purified by silica gel chromatography with 0·10% MeOH in DCM to afford the desired compound (35 mg product, 62.8%). LC/MS: 458.0 [M+H]⁺. ¹H NMR (400 MHz, DMSO-d6) δ 8.41 (s, 1H), 8.26 (d, J=5.3 Hz, 1H), 8.18 (s, 1H), 7.88 (d, J=8.8 Hz, 1H), 7.76 (d, J=8.8 Hz, 1H), 7.60 (t, J=7.3 Hz, 2H), 7.52 (t, J=7.1 Hz, 1H), 7.40-7.13 (m, 2H), 6.15 (d, J=5.2 Hz, 1H), 5.12-5.07 (m, 1H), 4.43 (d, J=12.1 Hz, 1H), 3.78 (d, J=10.7 Hz, 1H), 3.51 (t, J=11.7 Hz, 1H), 3.00 (t, J=11.9 Hz, 1H), 2.10-2.01 (m, 5H), 1.77 (t, J=11.5 Hz, 2H), 1.68 (d, J=6.8 Hz, 3H).

### Example 19: Preparation of (R)-1-(4-(4-((1-(3-(difluoromethyl)-2-fluorophenyl)ethyl)amino)quinolin-6-yl)-4-fluoropiperidin-1-yl)ethan-1-one (Compound 41)

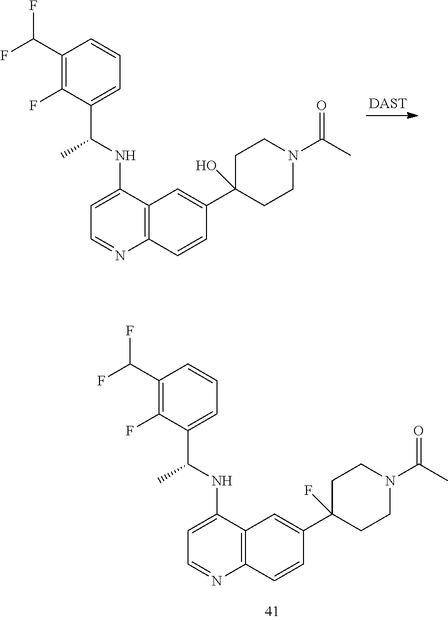

### Step 1: Preparation of (R)-1-(4-(4-((1-(3-(difluoromethyl)-2-fluorophenyl)ethyl)amino)quinolin-6-yl)-4-fluoropiperidin-1-yl)ethan-1-one (Compound 41)

To a solution of (R)-1-(4-(4-((1-(3-(difluoromethyl)-2-fluorophenyl)ethyl)amino)quinolin-6-yl)-4-hydroxypiperidin-1-yl)ethan-1-one (25 mg, 0.05 mmol) in DCM (3 mL) under argon at −78° C. was added DAST (17 mg, 0.1 mmol). The reaction was stirred at −78° C. for 30 minutes and then quenched with saturated sodium bicarbonate solution at room temperature. The mixture was extracted with EA. The organic phase was washed with brine and dried over Na₂SO₄. The solvent was removed in vacuum and the residue was purified by Prep-HPLC to afford the desired compound (9 mg, 35.8%). LC/MS: 460.1 [M+H]⁺. ¹H NMR (400 MHz, DMSO-d6) δ 8.84 (brs, 1H), 8.68 (s, 1H), 8.46 (d, J=6.4 Hz, 1H), 7.98 (d, J=10.2 Hz, 1H), 7.93 (d, J=8.8 Hz, 1H), 7.68 (t, J=7.1 Hz, 1H), 7.58 (t, J=6.9 Hz, 1H), 7.40 (s, 0.2H), 7.34 (t, J=7.7 Hz, 1H), 7.26 (s, 0.6H), 7.13 (s, 0.2H), 6.52 (d, J=6.3 Hz, 1H), 5.37-5.31 (m, 1H), 4.56 (d, J=8.4 Hz, 1H), 3.94 (d, J=10.3 Hz, 1H), 3.44 (t, J=11.9 Hz, 1H), 2.91 (t, J=11.9 Hz, 1H), 2.31-2.15 (m, 2H), 2.10 (s, 3H), 2.09-2.00 (m, 2H), 1.74 (d, J=6.7 Hz, 3H).

### Example 20: Preparation of (R)-1-(4-(4-((1-(3-(difluoromethyl)-2-fluorophenyl)ethyl)amino)-7-methoxyquinolin-6-yl)-4-methoxypiperidin-1-yl)ethan-1-one (Compound 73)

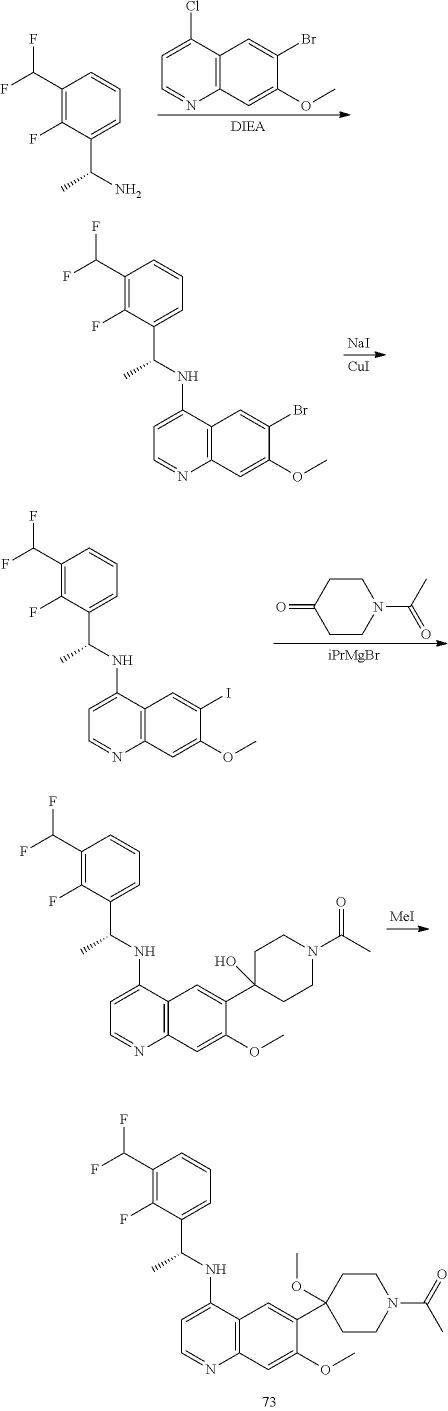

### Step 1: Preparation of (R)-6-bromo-N-(1-(3-(difluoromethyl)-2-fluorophenyl)ethyl)-7-methoxyquinolin-4-amine

To a solution of (R)-1-(3-(difluoromethyl)-2-fluorophenyl)ethan-1-amine (450 mg, 2.37 mmol) and 6-bromo-4-chloro-7-methoxyquinoline (777.9 mg, 2.85 mmol) in EtOH (10 mL) was added DIEA (403.23 mg, 3.12 mmol). The reaction mixture was stirred at 100° C. for 16 hours and then concentrated under vacuum. The residue was purified by flash chromatography (0-30% EA in PE) to give the desired product (400 mg, 80% purity, 39.6% yield) as a colorless oil. LC/MS: 425.0 [M+H]⁺.

### Step 2: Preparation of (R)—N-(1-(3-(difluoromethyl)-2-fluorophenyl)ethyl)-6-iodo-7-methoxy-quinolin-4-amine

To a solution of (R)-6-bromo-N-(1-(3-(difluoromethyl)-2-fluorophenyl)ethyl)-7-methoxyquinolin-4-amine (500 mg, 1.18 mmol) and DMEDA (41.6 mg, 0.47 mmol) in 1,4-dioxane (20 mL) was added Cul (44.7 mg, 0.24 mmol) and Nal (529.1 mg, 5.23 mmol). The reaction mixture was stirred at 110° C. for 2 hours and then concentrated under vacuum. The residue was purified by flash chromatography (0-50% EA in PE) to give the desired product (300 mg, 51.3%) as a yellow solid. LC/MS: 473.0 [M+H]⁺.

### Step 3: Preparation of (R)-1-(4-(4-((1-(3-(difluoromethyl)-2-fluorophenyl)ethyl)amino)-7-methoxyquinolin-6-yl)-4-hydroxypiperidin-1-yl)ethan-1-one

To a solution of (R)—N-(1-(3-(difluoromethyl)-2-fluorophenyl)ethyl)-6-iodo-7-methoxyquinolin-4-amine (400 mg, 0.84 mmol) and DMPU (217.1 mg, 1.68 mmol) in THF (10 mL) was added i-PrMgBr (249.5 mg, 1.69 mmol) at −20° C. The mixture was stirred at −20° C. for 1 hour and then 1-acetylpiperidin-4-one was added (143.5 mg, 1.02 mmol). The reaction mixture was stirred at 20° C. for 12 hours, and then quenched with aqueous NH₄Cl solution (20 mL) and extracted with EA (20 mL×2). The organic phase was concentrated in vacuum and the residue was purified by flash chromatography (0-40% EA in PE) to give the desired product (50 mg, 80% purity, 9.6% yield) as a white solid. LC/MS: 488.2 [M+H]⁺.

### Step 4: Preparation of (R)-1-(4-(4-((1-(3-(difluoromethyl)-2-fluorophenyl)ethyl)amino)-7-methoxyquinolin-6-yl)-4-methoxypiperidin-1-yl)ethan-1-one

To a solution of (R)-1-(4-(4-((1-(3-(difluoromethyl)-2-fluorophenyl)ethyl)amino)-7-methoxyquinolin-6-yl)-4-hydroxypiperidin-1-yl)ethan-1-one (40 mg, 0.08 mmol) in DMA (10 mL) was added Cs₂CO₃ (80.1 mg, 0.246 mmol) and CH₃I (11.6 mg, 0.082 mmol). The reaction mixture was stirred at 25° C. for 16 hours, and then quenched with H₂O (20 mL) and extracted with EA (15 mL). The organic phase was concentrated in vacuum and the residue was purified by Pre-TLC with PE:EA=1:1 to give the titled compound (10 mg, 24.2%) as a white solid. LC/MS: 501.9 [M+H]⁺. ¹H NMR (400 MHz, CD₃OD) δ 8.78 (s, 1H), 8.31 (d, J=7.4 Hz, 1H), 7.64 (t, J=7.3 Hz, 1H), 7.57 (t, J=7.0 Hz, 1H), 7.34-7.27 (m, 2H), 7.03 (t, J=54.7 Hz, 1H), 6.59 (dd, J=7.5, 1.8 Hz, 1H), 5.46-5.38 (m, 1H), 4.54-4.45 (m, 1H), 4.12 (d, J=2.7 Hz, 6H), 3.92-3.83 (m, 1H), 3.74-3.64 (m, 1H), 3.23-3.13 (m, 1H), 2.63-2.47 (m, 2H), 2.18 (s, 3H), 1.82 (d, J=6.8 Hz, 3H), 1.81-1.71 (m, 2H).

### Example 21: Preparation of 1-((R)-3-(4-(((R)-1-(3-(difluoromethyl)-2-fluorophenyl)ethyl)amino)quinolin-6-yl)-3-methoxypyrrolidin-1-yl)ethan-1-one (Compound 75) and 1-((S)-3-(4-(((R)-1-(3-(difluoromethyl)-2-fluorophenyl)ethyl)amino)quinolin-6-yl)-3-methoxypyrrolidin-1-yl)ethan-1-one (Compound 76)

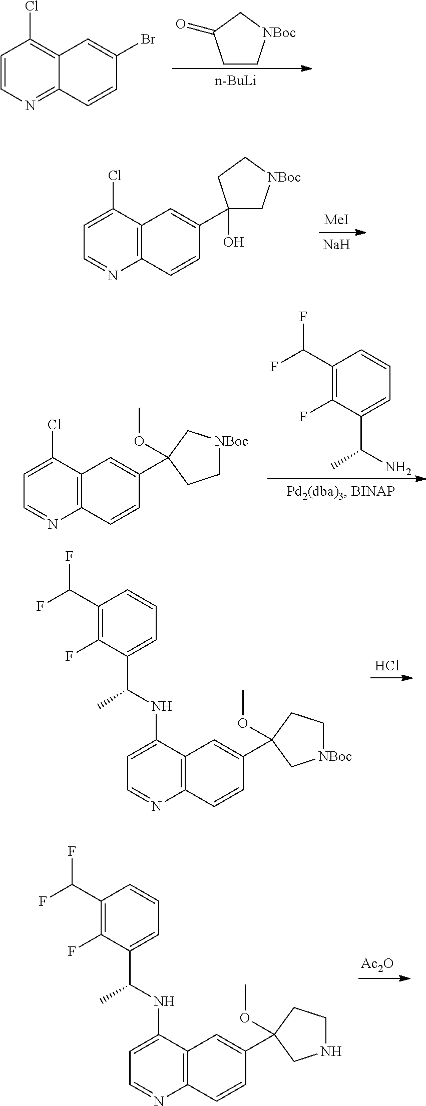

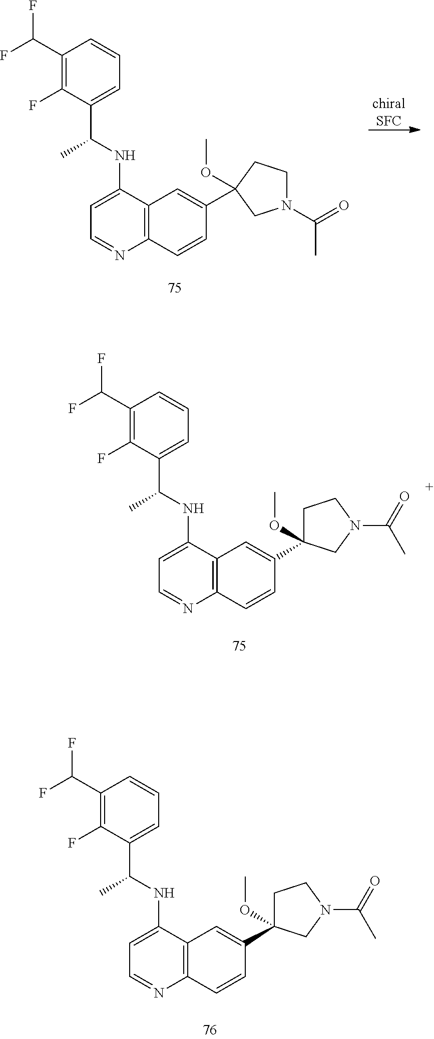 [SMILES: COC1(c2ccc3nccc(N[C@H](C)c4cccc(C(F)F)c4F)c3c2)CCN(C(C)=O)C1 ; "chiralSFC" ; CO[C@@]1(c2ccc3nccc(N[C@H](C)c4cccc(C(F)F)c4F)c3c2)CCN(C(C)=O)C1 ; CO[C@]1(c2ccc3nccc(N[C@H](C)c4cccc(C(F)F)c4F)c3c2)CCN(C(C)=O)C1]

### Step 1: Preparation of tert-butyl 3-(4-chloroquinolin-6-yl)-3-hydroxypyrrolidine-1-carboxylate

To a solution of 6-bromo-4-chloroquinoline (1 g, 4.12 mmol) in THE (100 mL) stirred under argon at −80° C. was added n-BuLi (2 mL, 4.95 mmol). The reaction mixture was stirred at −80° C. for 1 hour and a solution of tert-butyl 3-oxopyrrolidine-1-carboxylate (1.53 g, 5.25 mmol) in THF (20 mL) was added. The reaction mixture was stirred at −80° C. for 1 hour and was quenched with water and extracted with EA. The organic phase was washed with brine and dried over Na₂SO₄. The solvent was removed under vacuum and the residue was purified by silica gel chromatography using 0·10% MeOH in DCM to afford the desired compound (800 mg, 55.6%) as a yellow solid. LC/MS: 349.2 [M+H]⁺.

### Step 2: Preparation of tert-butyl 3-(4-chloroquinolin-6-yl)-3-methoxypyrrolidine-1-carboxylate

To a solution of tert-butyl 3-(4-chloroquinolin-6-yl)-3-hydroxypyrrolidine-1-carboxylate (700 mg, 2.00 mmol) in DMF (10 mL) was added NaH (240 mg, 6.02 mmol) and CH₃I (1.4 g, 10.03 mmol) at 0° C. The reaction was stirred under nitrogen at 25° C. for 2 hours. The mixture was diluted with water (30 mL) and extracted with EA (30 mL×2). The organic phase was washed with brine and dried over Na₂SO₄. The solvent was removed in vacuum and the residue was purified by flash with PE:EA=1:1 to afford the desired product (500 mg, 68.8%) as a yellow solid. LC/MS: 363.1 [M+H]⁺.

### Step 3: Preparation of tert-butyl 3-(4-(((R)-1-(3-(difluoromethyl)-2-fluorophenyl) ethyl)amino) quinolin-6-yl)-3-methoxypyrrolidine-1-carboxylate

To a solution of tert-butyl 3-(4-chloroquinolin-6-yl)-3-methoxypyrrolidine-1-carboxylate (500 mg, 1.37 mmol) in dioxane (20 mL) was added (R)-1-(3-(difluoromethyl)-2-fluorophenyl)ethan-1-amine (286 mg, 1.51 mmol), Pd₂(dba)₃ (504 mg, 0.55 mmol), BINAP (343 mg, 0.55 mmol), and t-BuONa (397 mg, 4.13 mmol). The reaction mixture was stirred under nitrogen at 110° C. for 2 hours. The solvent was removed in vacuum and the residue was purified by flash with PE:EA=1:1 to afford the desired product (300 mg, 42.1%) as a yellow solid. LC/MS: 516.3 [M+H]⁺.

### Step 4: Preparation of N—((R)-1-(3-(difluoromethyl)-2-fluorophenyl)ethyl)-6-(3-methoxypyrrolidin-3-yl) quinolin-4-amine

A solution of tert-butyl 3-(4-(((R)-1-(3-(difluoromethyl)-2-fluorophenyl) ethyl)amino)quinolin-6-yl)-3-methoxypyrrolidine-1-carboxylate (150 mg, 0.29 mmol) in HCl/dioxane (4 N, 10 mL) was stirred at room temperature for 2 hours. The solvent was removed in vacuum to afford the desired compound (150 mg of HCl salt, crude) as a yellow solid. LC/MS: 415.9 [M+H]⁺.

### Step 5: Preparation of 1-(3-(4-(((R)-1-(3-(difluoromethyl)-2-fluorophenyl)ethyl)amino)quinolin-6-yl)-3-methoxypyrrolidin-1-yl)ethan-1-one

To a solution of N—((R)-1-(3-(difluoromethyl)-2-fluorophenyl)ethyl)-6-(3-methoxypyrrolidin-3-yl)quinolin-4-amine (100 mg, 0.24 mmol) in DCM (10 mL) was added TEA (73.1 mg, 0.72 mmol) and Ac₂O (49.1 mg, 0.48 mmol) under nitrogen. The reaction was stirred at 25° C. for 2 hours. The mixture was diluted with water (30 mL) and extracted with DCM (20 mL×2). The organic phase was washed with brine and dried over Na₂SO₄. The solvent was removed in vacuum and the residue was purified by flash with DCM: MeOH=10:1 to afford the desired product (70 mg, 63.6%) as a yellow solid. LC/MS: 458.2 [M+H]⁺.

### Step 6: Preparation of 1-((R)-3-(4-(((R)-1-(3-(difluoromethyl)-2-fluorophenyl)ethyl)amino)quinolin-6-yl)-3-methoxypyrrolidin-1-yl)ethan-1-one (Compound 75) and 1-((S)-3-(4-(((R)-1-(3-(difluoromethyl)-2-fluorophenyl)ethyl)amino)quinolin-6-yl)-3-methoxypyrrolidin-1-yl)ethan-1-one (Compound 76)

The racemic sample of 1-(3-(4-(((R)-1-(3-(difluoromethyl)-2-fluorophenyl)ethyl)amino)quinolin-6-yl)-3-methoxy pyrrolidin-1-yl)ethan-1-one (70 mg, 0.15 mmol) was separated with SFC Thar prep 80 via CHIRALPAK IC 250 mm×20 mm, 5 μm, 40% ETOH (NH4OH 0.2%) in CO₂ (Isocratic) for 18 mins to afford the desired compound 75 (Rt=10.93 min, 11.3 mg, 16.4%) and compound 76 (Rt=14.44 min, 10.8 mg, 14.3%).

Analytical SFC conditions: SFC Thar X-5 via CHIRALPAK IC 250 mm×4.6 mm, 5 μm, 40% ETOH (NH4OH 0.2%) in CO₂ (Isocratic). Compound 75: LC/MS: 458.2 [M+H]⁺. ¹H NMR (400 MHz, DMSO) δ 8.90-8.79 (m, 1H), 8.70-8.62 (m, 1H), 8.46 (d, J=6.4 Hz, 1H), 7.92 (s, 2H), 7.73-7.65 (m, 1H), 7.58 (t, J=7.2 Hz, 1H), 7.37-7.13 (m, 2H), 6.54-6.47 (m, 1H), 5.38-5.31 (m, 1H), 4.24-4.04 (m, 1H), 3.79-3.71 (m, 1H), 3.66-3.60 (m, 1H), 3.53 (d, J=12.8 Hz, 1H), 3.01-2.93 (m, 3H), 2.67-2.57 (m, 1H), 2.43-2.34 (m, 1H), 2.05-1.99 (m, 3H), 1.75 (d, J=6.8 Hz, 3H). SFC Rt=5.79. Compound 76: LC/MS: 458.2 [M+H]⁺. ¹H NMR (400 MHz, DMSO) δ 8.87-8.76 (m, 1H), 8.66-8.60 (m, 1H), 8.46 (d, J=6.5 Hz, 1H), 7.91 (s, 2H), 7.70-7.64 (m, 1H), 7.58 (t, J=6.9 Hz, 1H), 7.37-7.13 (m, 2H), 6.53-6.46 (m, 1H), 5.37-5.30 (m, 1H), 4.23-4.03 (m, 1H), 3.77-3.70 (m, 1H), 3.67-3.60 (m, 1H), 3.54 (d, J=12.6 Hz, 1H), 3.01-2.93 (m, 3H), 2.67-2.58 (m, 1H), 2.43-2.36 (m, 1H), 2.03-1.99 (m, 3H), 1.75 (d, J=6.7 Hz, 3H). SFC Rt=7.36

The compounds in Table 3 were prepared analogously according to the procedures described for Examples 1 to 21.

TABLE 3
Additional Compounds Prepared According to Procedures Described for
Examples 1 to 21.

| Cpd # | Structure | Salt | Analytical Data |
|---|---|---|---|
| 19 | 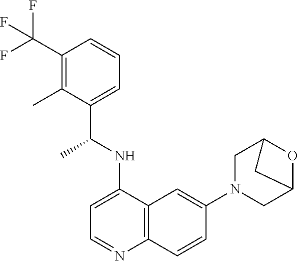 [SMILES: Cc1c([C@@H](C)Nc2ccnc3ccc(N4CC5CC(C4)O5)cc23)cccc1C(F)(F)F] | TFA | LC/MS: 428.1 [M + H]⁺. ¹H NMR (400 MHz, DMSO) δ 8.05 (d, J = 5.2 Hz, 1H), 7.71 (d, J = 9.0 Hz, 1H), 7.67 (d, J = 7.4 Hz, 1H), 7.57 (d, J = 7.6 Hz, 1H), 7.44-7.27 (m, 4H), 5.95 (d, J = 5.6 Hz, 1H), 5.09 (t, J = 8.0 Hz, 1H), 4.81 (d, J = 6.4 Hz, 2H), 3.85- 3.72 (m, 2H), 3.62-3.53 (m, 2H), 3.21- 3.16 (m, 1H), 2.58 (s, 3H), 2.01 (d, J = 8.8 Hz, 1H), 1.62 (d, J = 6.8 Hz, 3H). |
| 20 | 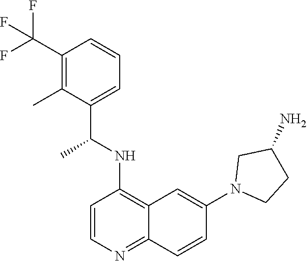 [SMILES: Cc1c([C@@H](C)Nc2ccnc3ccc(N4CC[C@@H](N)C4)cc23)cccc1C(F)(F)F] | HCl | LCMS: 415.1 [M + H]⁺. ¹H NMR (400 MHz, DMSO) δ 13.93 (s, 1H), 8.90 (d, J = 8.0 Hz, 1H), 8.29- 8.22 (m, 3H), 7.81 (d, J = 9.2 Hz, 1H), 7.64 (d, J = 8.0 Hz, 2H), 7.52-7.42 (m, 2H), 7.39 (t, J = 7.8 Hz, 1H), 6.32 (d, J = 7.2 Hz, 1H), 5.41-5.33 (m, 1H), 4.12- 4.04 (m, 1H), 3.77-3.66 (m, 2H), 3.54-3.41 (m, 4H), 2.46-2.37 (m, 1H), 2.22-2.13 (m, 1H), 1.70 (d, J = 6.8 Hz, 3H). |
| 21 | 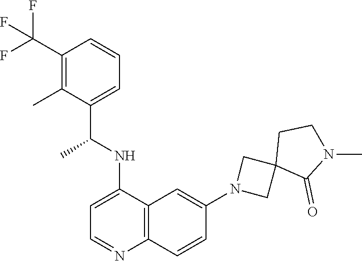 [SMILES: Cc1c([C@@H](C)Nc2ccnc3ccc(N4CC5(CCN(C)C5=O)C4)cc23)cccc1C(F)(F)F] | | LCMS: 469.1 [M + H]⁺. ¹H NMR (400 MHz, DMSO) δ 8.80- 8.76 (m, 1H), 8.25 (d, J = 6.7 Hz, 1H), 7.82-7.77 (m, 1H), 7.70-7.61 (m, 2H), 7.54-7.50 (m, 1H), 7.38 (t, J = 7.8 Hz, 1H), 7.23 (dd, J = 9.0, 2.2 Hz, 1H), 6.26 (d, J = 6.8 Hz, 1H), 5.32 (t, J = 6.6 Hz, 1H), 4.13-4.07 (m, 2H), 4.03- 3.98 (m, 2H), 3.38-3.35 (m, 2H), 2.79 (s, 3H), 2.57 (s, 3H), 2.45 (t, J = 6.8 Hz, 2H), 1.68 (d, J = 6.7 Hz, 3H). |
| 22 | 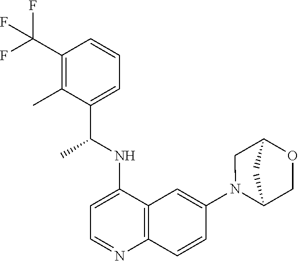 [SMILES: Cc1c([C@@H](C)Nc2ccnc3ccc(N4C[C@@H]5C[C@H]4CO5)cc23)cccc1C(F)(F)F] | | LC/MS: 428.2 [M + H]⁺. ¹H NMR (400 MHz, DMSO) δ 8.03 (d, J = 5.4 Hz, 1H), 7.69-7.62 (m, 2H), 7.57 (d, J = 7.6 Hz, 1H), 7.34-7.31 (m, 3H), 7.22 (dd, J = 9.2, 2.4 Hz, 1H), 5.92 (d, J = 5.5 Hz, 1H), 5.11-5.05 (m, 1H), 4.90-4.83 (m, 1H), 4.74-4.69 (m, 1H), 3.85 (d, J = 6.6 Hz, 1H), 3.72 (d, J = 7.4 Hz, 1H), 3.62 (d, J = 8.5 Hz, 1H), 3.24 (d, J = 9.7 Hz, 1H), 2.57 (s, 3H), 2.04-1.92 (m, 2H), 1.61 (d, J = 6.7 Hz, 3H). |
| 23 | 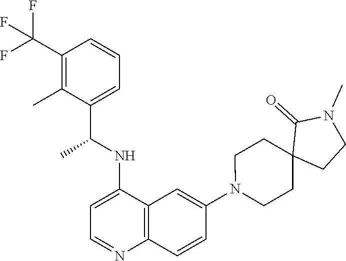 [SMILES: Cc1c([C@@H](C)Nc2ccnc3ccc(N4CCC5(CCN(C)C5=O)CC4)cc23)cccc1C(F)(F)F] | | LC/MS: 497.3 [M + H]⁺. ¹H NMR (400 MHz, DMSO) δ 8.90 (brs, 1H), 8.23 (d, J = 6.5 Hz, 1H), 7.91- 7.72 (m, 4H), 7.62 (d, J = 8.2 Hz, 1H), 7.38 (t, J = 7.5 Hz, 1H), 6.23 (d, J = 6.8 Hz, 1H), 5.32-5.28 (m, 1H), 4.04- 3.95 (m, 2H), 3.09-3.01 (m, 2H), 2.76 (s, 3H), 2.58 (s, 3H), 2.06-1.99 (m, 2H), 1.93-1.78 (m, 3H), 1.70 (d, J = 6.6 Hz, 3H), 1.56-1.48 (m, 2H), 1.26- 1.21 (m, 1H). |
| 24 | 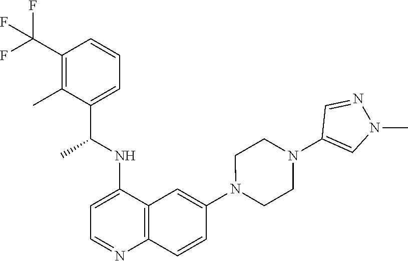 [SMILES: Cc1c([C@@H](C)Nc2ccnc3ccc(N4CCN(c5cnn(C)c5)CC4)cc23)cccc1C(F)(F)F] | FA | LC/MS: 495.0 [M + H]⁺. ¹H NMR (400 MHz, ) δ 8.21 (brs, 1H), 8.04 (d, J = 5.2 Hz, 1H), 7.64-7.61 (m, 3H), 7.52 (d, J = 7.2 Hz, 1H), 7.48 (dd, J = 9.2, 2.4 Hz, 1H), 7.33 (s, 1H), 7.30- 7.25 (m, 2H), 7.21 (s, 1H), 5.88 (d, J = 5.4 Hz, 1H), 5.05-4.97 (m, 1H), 3.72 (s, 3H), 3.44-3.37 (m, 4H), 3.05-3.01 (m, 4H), 2.54 (s, 3H), 1.57 (d, J = 6.7 Hz, 3H). |
| 25 | 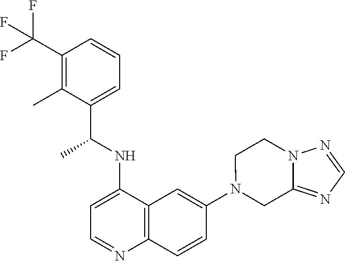 [SMILES: Cc1c([C@@H](C)Nc2ccnc3ccc(N4CCn5ncnc5C4)cc23)cccc1C(F)(F)F] | FA | LC/MS: 453.2 [M + H]⁺. ¹H NMR (400 MHz, DMSO) δ 8.17 (brs, 1H), 8.13 (d, J = 5.4 Hz, 1H), 8.04 (s, 1H), 7.78-7.66 (m, 4H), 7.58 (d, J = 7.5 Hz, 1H), 7.53-7.48 (m, 1H), 7.33 (t, J = 7.8 Hz, 1H), 5.98 (d, J = 5.5 Hz, 1H), 5.12-5.05 (m, 1H), 4.75 (d, J = 3.4 Hz, 2H), 4.38 (t, J = 5.2 Hz, 2H), 3.99 (t, J = 5.4 Hz, 2H), 2.58 (s, 3H), 1.64 (d, J = 6.7 Hz, 3H). |
| 26 | 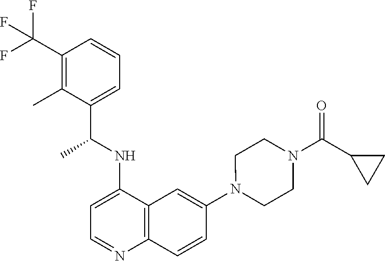 [SMILES: Cc1c([C@@H](C)Nc2ccnc3ccc(N4CCN(C(=O)C5CC5)CC4)cc23)cccc1C(F)(F)F] | | LC/MS: 483.0 [M + H]⁺. ¹H NMR (400 MHz, DMSO) δ 8.69 (brs, 1H), 8.25 (d, J = 6.5 Hz, 1H), 7.91- 7.87 (m, 1H), 7.84-7.79 (m, 1H), 7.78-7.73 (m, 1H), 7.70 (d, J = 7.8 Hz, 1H), 7.62 (d, J = 7.6 Hz, 1H), 7.37 (t, J = 7.8 Hz, 1H), 6.22 (d, J = 6.7 Hz, 1H), 5.32-5.26 (m, 1H), 3.97-3.87 (m, 2H), 3.76-3.67 (m, 2H), 3.50-3.38 (m, 4H), 2.58 (s, 3H), 2.12-2.06 (m, 1H), 1.69 (d, J = 6.7 Hz, 3H), 0.81- 0.73 (m, 4H). |
| 27 | 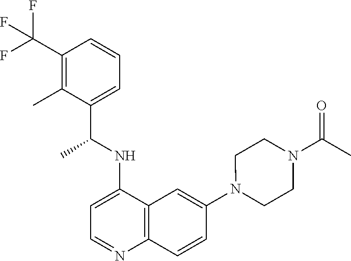 [SMILES: CC(=O)N1CCN(c2ccc3nccc(N[C@H](C)c4cccc(C(F)(F)F)c4C)c3c2)CC1] | FA | LC/MS: 456.9 [M + H]⁺. ¹H NMR (400 MHz, DMSO) δ 8.27 (brs, 1H), 8.09 (d, J = 5.3 Hz, 1H), 7.70- 7.63 (m, 3H), 7.57 (d, J = 7.6 Hz, 1H), 7.50 (dd, J = 9.2, 2.4 Hz, 1H), 7.36- 7.28 (m, 2H), 5.93 (d, J = 5.4 Hz, 1H), 5.09-5.00 (m, 1H), 3.72-3.65 (m, 4H), 3.38-3.24 (m, 4H), 2.57 (s, 3H), 2.09 (s, 3H), 1.61 (d, J = 6.7 Hz, 3H). |
| 28 | 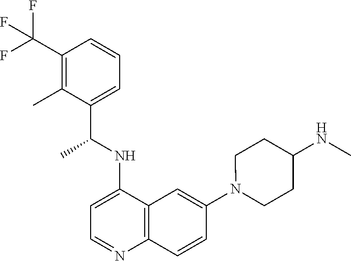 [SMILES: CNC1CCN(c2ccc3nccc(N[C@H](C)c4cccc(C(F)(F)F)c4C)c3c2)CC1] | FA | LC/MS: 443.2 [M + H]⁺. ¹H NMR (400 MHz, DMSO) δ 8.33 (brs, 2H), 8.07 (d, J = 5.2 Hz, 1H), 7.72- 7.61 (m, 3H), 7.56 (d, J = 7.7 Hz, 1H), 7.47 (dd, J = 9.3, 1.5 Hz, 1H), 7.34- 7.29 (m, 2H), 5.91 (d, J = 5.5 Hz, 1H), 5.04 (t, J = 6.6 Hz, 1H), 4.02-3.95 (m, 2H), 3.10-3.01 (m, 1H), 2.90-2.78 (m, 2H), 2.57 (s, 3H), 2.54 (s, 3H), 2.18- 2.06 (m, 2H), 1.66-1.59 (m, 5H). |
| 29 | 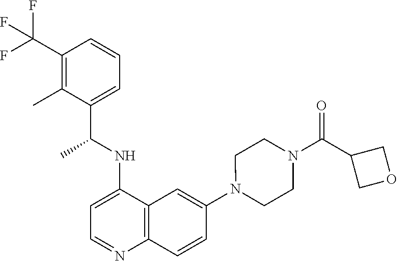 [SMILES: Cc1c([C@@H](C)Nc2ccnc3ccc(N4CCN(C(=O)C5COC5)CC4)cc23)cccc1C(F)(F)F] | FA | LCMS: 499.2 [M + H]⁺. ¹H NMR (400 MHz, DMSO) δ 8.18 (d, J = 6.0 Hz, 1H), 8.15 (s, 0.5 H), 7.75- 7.72 (m, 2H), 7.68-7.58 (m, 3H), 7.35 (t, J = 7.7 Hz, 1H), 6.11 (d, J = 6.1 Hz, 1H), 5.22-5.14 (m, 1H), 4.77-4.70 (m, 4H), 4.26-4.18 (m, 1H), 3.73 (t, J = 5.0 Hz, 2H), 3.38-3.31 (m, 6H), 2.57 (s, 3H), 2.55 (s, 1H), 1.65 (d, J = 6.7 Hz, 3H). |
| 30 | 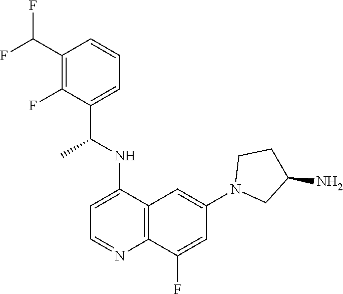 [SMILES: C[C@@H](Nc1ccnc2c(F)cc(N3CC[C@@H](N)C3)cc12)c1cccc(C(F)F)c1F] | FA | LC/MS: 419.1 [M + H]⁺. ¹H NMR (400 MHz, MeOD) δ 8.50 (brs, 3H), 8.05 (d, J = 5.7 Hz, 1H), 7.53 (dd, J = 15.4, 7.4 Hz, 2H), 7.23 (t, J = 7.8 Hz, 1H), 7.18-6.91(m, 3H), 6.27 (d, J = 5.8 Hz, 1H), 5.37-5.32 (m, 1H), 5.24- 5.18 (m, 1H), 4.07-4.02 (m, 1H), 3.78-3.73 (m, 1H), 3.57-3.51 (m, 1H), 2.56-2.48 (m, 1H), 2.21-2.17 (m, 1H), 2.05-2.01 (m, 1H), 1.77 (d, J = 6.8 Hz, 3H). |
| 31 | 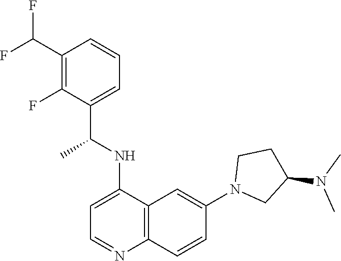 [SMILES: C[C@@H](Nc1ccnc2ccc(N3CC[C@@H](N(C)C)C3)cc12)c1cccc(C(F)F)c1F] | FA | LC/MS: 429.2 [M + H]⁺. ¹H NMR (400 MHz, DMSO) δ 8.23 (s, 2H), 8.05 (d, J = 5.5 Hz, 1H), 7.67 (d, J = 9.1 Hz, 1H), 7.58-7.51 (m, 2H), 7.41- 7.25 (m, 3H), 7.20-7.13 (m, 2H), 6.11 (d, J = 5.5 Hz, 1H), 5.18-5.10 (m, 1H), 3.68-3.53 (m, 2H), 3.45-3.36 (m, 1H), 3.25-3.17 (m, 1H), 2.98- 2.90 (m, 1H), 2.31-2.21 (m, 7H), 1.98- 1.86 (m, 1H), 1.69 (d, J = 6.8 Hz, 3H). |
| 32 | 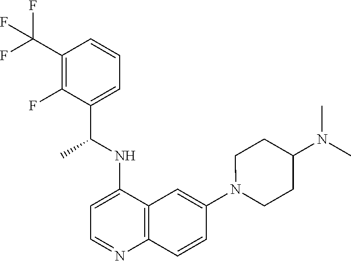 | FA | LC/MS: 457.2 [M + H]⁺. ¹H NMR (400 MHz, DMSO) δ 8.23- 8.19 (m, 2H), 8.07 (d, J = 5.4 Hz, 1H), 7.68-7.62 (m, 3H), 7.58-7.55 (m, 1H), 7.50-7.47 (m, 1H), 7.40-7.35 (m, 1H), 7.34-7.30 (m, 1H), 5.92 (d, J = 5.1 Hz, 1H), 5.07-5.02 (m, 1H), 4.00- 3.94 (m, 2H), 2.85-2.77 (m, 2H), 2.57 (s, 3H), 2.49-2.44 (m, 1H), 2.34 (d, J = 3.1 Hz, 6H), 1.99-1.92 (m, 2H), 1.67-1.56 (m, 5H). |
| 33 | 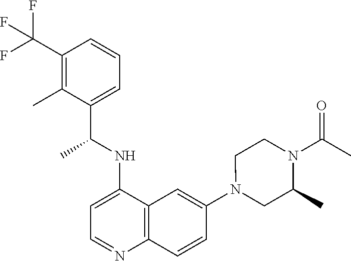 [SMILES: CC(=O)N1CCN(c2ccc3nccc(N[C@H](C)c4cccc(C(F)(F)F)c4C)c3c2)C[C@@H]1C] | FA | LCMS: 470.9 [M + H]⁺. ¹H NMR (400 MHz, DMSO) δ 8.26 (s, 1H), 8.07 (d, J = 5.2 Hz, 1H), 7.66 (d, J = 9.2 Hz, 2H), 7.63-7.54 (m, 2H), 7.47 (d, J = 9.2 Hz, 1H), 7.34-7.23 (m, 2H), 5.91 (d, J = 5.3 Hz, 1H), 5.08-5.00 (m, 1H), 4.41-4.33 (m, 1H), 3.91-3.87 (m, 2H), 3.74-3.70 (m, 2H), 3.10- 3.03 (m, 2H), 2.57 (s, 3H), 2.08 (d, J = 11.8 Hz, 3H), 1.61 (d, J = 6.7 Hz, 3H), 1.41-1.23 (m, 3H). |
| 34 | 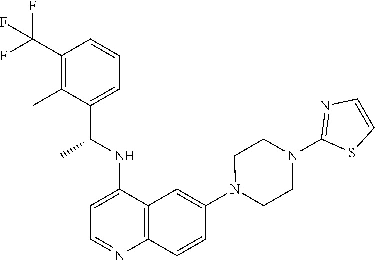 [SMILES: Cc1c([C@@H](C)Nc2ccnc3ccc(N4CCN(c5nccs5)CC4)cc23)cccc1C(F)(F)F] | FA | LC/MS: 498.2 [M + H]⁺. ¹H NMR (400 MHz, DMSO) δ 8.18 (s, 1H), 8.10 (d, J = 5.3 Hz, 1H), 7.74- 7.65 (m, 3H), 7.59-7.52 (m, 2H), 7.39 (d, J = 6.3 Hz, 1H), 7.32 (t, J = 7.9 Hz, 1H), 7.23 (d, J = 3.6 Hz, 1H), 6.92 (d, J = 3.6 Hz, 1H), 5.94 (d, J = 5.5 Hz, 1H), 5.13-5.00 (m, 1H), 3.68-3.60 (m, 4H), 3.53-3.42 (m, 4H), 2.58 (s, 3H), 1.62 (d, J = 6.7 Hz, 3H). |
| 35 | 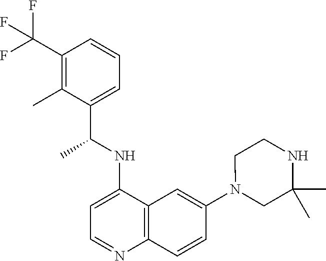 [SMILES: Cc1c([C@@H](C)Nc2ccnc3ccc(N4CCNC(C)(C)C4)cc23)cccc1C(F)(F)F] | FA | LC/MS: 443.1 [M + H]⁺. ¹H NMR (400 MHz, DMSO) δ 8.29 (brs, 2H), 8.10 (d, J = 5.2 Hz, 1H), 7.68- 7.60 (m, 3H), 7.57 (d, J = 7.7 Hz, 1H), 7.49 (d, J = 8.6 Hz, 1H), 7.38 (d, J = 5.8 Hz, 1H), 7.32 (t, J = 7.7 Hz, 1H), 5.93 (d, J = 5.4 Hz, 1H), 5.10-5.01 (m, 1H), 3.51-3.33 (m, 2H), 3.32-3.15 (m, 4H), 2.58 (s, 3H), 1.62 (d, J = 6.6 Hz, 3H), 1.37 (s, 6H). |
| 36 | 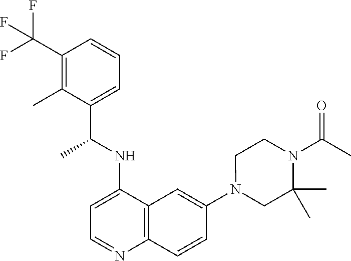 [SMILES: CC(=O)N1CCN(c2ccc3nccc(N[C@H](C)c4cccc(C(F)(F)F)c4C)c3c2)CC1(C)C] | TFA | LC/MS: 485.1 [M + H]⁺. ¹H NMR (400 MHz, DMSO) δ 13.79 (s, 1H), 8.80 (d, J = 5.9 Hz, 1H), 8.25 (t, J = 6.4 Hz, 1H), 7.80-7.72 (m, 1H), 7.72- 7.59 (m, 3H), 7.50 (s, 1H), 7.39 (t, J = 7.8 Hz, 1H), 6.27 (d, J = 6.9 Hz, 1H), 5.41-5.29 (m, 1H), 3.90-3.83 (m, 2H), 3.72-3.69 (m, 2H), 3.64-3.57 (m, 2H), 2.58 (s, 3H), 2.06 (s, 3H), 1.70 (d, J = 6.6 Hz, 3H), 1.44 (d, J = 2.1 Hz, 6H). |
| 37 | 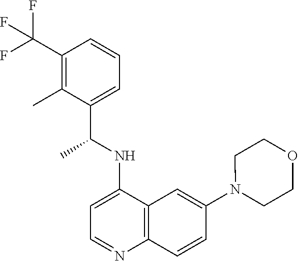 [SMILES: Cc1c([C@@H](C)Nc2ccnc3ccc(N4CCOCC4)cc23)cccc1C(F)(F)F] | FA | LC/MS: 416.2 [M + H]⁺. ¹H NMR (400 MHz, DMSO) δ 8.18 (s, 1H), 8.11 (d, J = 5.5 Hz, 1H), 7.72- 7.63 (m, 3H), 7.57 (d, J = 7.6 Hz, 1H), 7.54-7.43 (m, 2H), 7.32 (t, J = 7.8 Hz, 1H), 5.97 (d, J = 5.6 Hz, 1H), 5.12- 5.04 (m, 1H), 3.86-3.81 (m, 4H), 3.35- 3.26 (m, 4H), 2.57 (s, 3H), 1.61 (d, J = 6.7 Hz, 3H). |
| 38 | 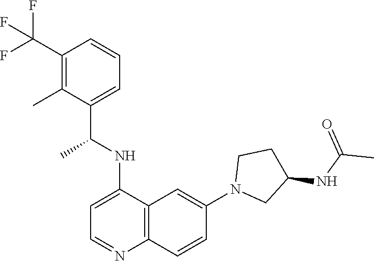 [SMILES: CC(=O)N[C@@H]1CCN(c2ccc3nccc(N[C@H](C)c4cccc(C(F)(F)F)c4C)c3c2)C1] | TFA | LCMS: 457.2 [M + H]⁺. ¹H NMR (400 MHz, DMSO) δ 8.28 (s, 1H), 8.23 (d, J = 7.0 Hz, 1H), 7.99 (d, J = 5.2 Hz, 1H), 7.67-7.62 (m, 2H), 7.56 (d, J = 7.6 Hz, 1H), 7.31 (t, J = 7.6 Hz, 1H), 7.24-7.19 (m, 1H), 7.18-7.10 (m, 2H), 5.89 (d, J = 5.2 Hz, 1H), 5.08- 5.02 (m, 1H), 4.48-4.43 (m, 1H), 3.72- 3.67 (m, 1H), 3.60-3.55 (m, 1H), 3.47-3.41 (m, 1H), 3.24 (dd, J = 10.0, 4.4 Hz, 1H), 2.57 (s, 3H), 2.28-2.21 (m, 1H), 1.99-1.92 (m, 1H), 1.84 (s, 3H), 1.60 (d, J = 6.8 Hz, 3H). |
| 39 | 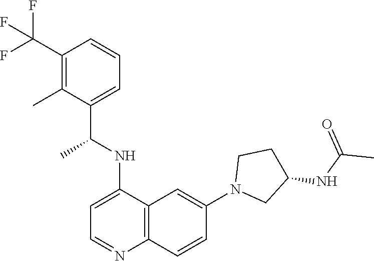 [SMILES: CC(=O)N[C@H]1CCN(c2ccc3nccc(N[C@H](C)c4cccc(C(F)(F)F)c4C)c3c2)C1] | TFA | LCMS: 457.2 [M + H]⁺. ¹H NMR (400 MHz, DMSO) δ 8.27- 8.22 (m, 2H), 8.01 (d, J = 5.2 Hz, 1H), 7.68-7.62 (m, 2H), 7.56 (d, J = 7.6 Hz, 1H), 7.38-7.28 (m, 2H), 7.20-7.12 (m, 2H), 5.92 (d, J = 5.6 Hz, 1H), 5.10- 5.04 (m, 1H), 4.50-4.43 (m, 1H), 3.66- 3.62 (m, 1H), 3.58-3.45 (m, 2H), 3.31-3.25 (m, 1H), 2.57 (s, 3H), 2.28- 2.20 (m, 1H), 2.01-1.93 (m , 1H), 1.85 (s, 3H), 1.61 (d, J = 6.8 Hz, 3H). |
| 42 | 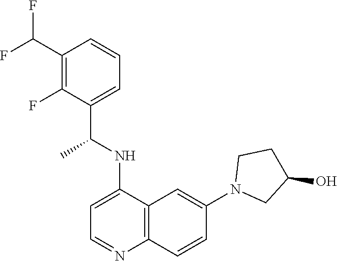 [SMILES: C[C@@H](Nc1ccnc2ccc(N3CC[C@@H](O)C3)cc12)c1cccc(C(F)F)c1F] | | LC/MS: 402.0 [M + H]⁺. ¹H NMR (400 MHz, DMSO) δ 8.22 (s, 1H), 8.04 (d, J = 5.4 Hz, 1H), 7.65 (d, J = 9.8 Hz, 1H), 7.58 (t, J = 7.2 Hz, 1H), 7.52 (t, J = 7.0 Hz, 1H), 7.42-7.22 (m, 3H), 7.17-7.12 (m, 2H), 6.09 (d, J = 5.5 Hz, 1H), 5.16-5.09 (m, 1H), 4.51- 4.47 (m, 1H), 3.63-3.42 (m, 3H), 3.34- 3.26 (m, 1H), 2.17-2.08 (m, 1H), 2.01-1.93 (m, 1H), 1.68 (d, J = 6.8 Hz, 3H). |
| 43 | 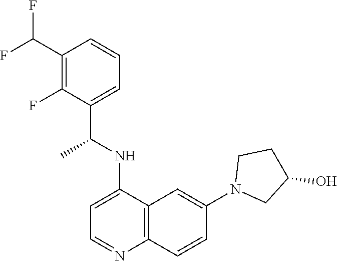 [SMILES: C[C@@H](Nc1ccnc2ccc(N3CC[C@H](O)C3)cc12)c1cccc(C(F)F)c1F] | | LC/MS: 402.0 [M + H]⁺. ¹H NMR (400 MHz, DMSO) δ 8.22 (s, 1H), 8.04 (d, J = 5.3 Hz, 1H), 7.66 (d, J = 8.7 Hz, 1H), 7.58 (t, J = 7.4 Hz, 1H), 7.52 (t, J = 6.9 Hz, 1H), 7.42-7.23 (m, 3H), 7.17-7.12 (m, 2H), 6.13-6.07 (m, 1H), 5.17-5.10 (m, 1H), 4.51- 4.47 (m, 1H), 3.63-3.43 (m, 3H), 3.34- 3.26 (m, 1H), 2.18-2.08 (m, 1H), 2.01-1.93 (m, 1H), 1.69 (d, J = 6.7 Hz, 3H). |
| 44 | 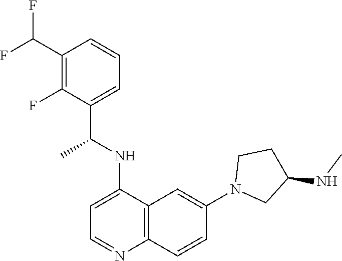 [SMILES: CN[C@@H]1CCN(c2ccc3nccc(N[C@H](C)c4cccc(C(F)F)c4F)c3c2)C1] | TFA | LC/MS: 415.2 [M + H]⁺. ¹H NMR (400 MHz, DMSO) δ 8.90 (brs, 1H), 8.82 (d, J = 6.9 Hz, 1H), 8.31 (d, J = 6.8 Hz, 1H), 7.81 (d, J = 9.0 Hz, 1H), 7.65-7.60 (m, 2H), 7.49-7.43 (m, 2H), 7.39-7.12 (m, 2H), 6.55- 6.53 (m, 1H), 5.47-5.39 (m, 1H), 4.03- 3.99 (m, 1H), 3.79-3.74 (m, 1H), 3.69-3.58 (m, 2H), 3.51-3.44 (m, 1H), 2.69 (s, 3H), 2.47-2.40 (m, 1H), 2.31-2.22 (m, 1H), 1.76 (d, J = 6.7 Hz, 3H). |
| 45 | 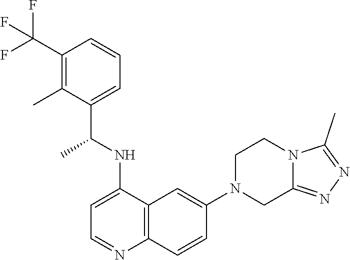 [SMILES: Cc1c([C@@H](C)Nc2ccnc3ccc(N4CCn5c(C)nnc5C4)cc23)cccc1C(F)(F)F] | FA | LC/MS: 467.0 [M + H]⁺. ¹H NMR (400 MHz, DMSO) δ 8.21 (s, 1H), 8.10 (d, J = 5.2 Hz, 1H), 7.74 (d, J = 2.3 Hz, 1H), 7.72-7.65 (m, 2H), 7.65- 7.59 (m, 1H), 7.57 (d, J = 7.5 Hz, 1H), 7.39-7.28 (m, 2H), 5.93 (d, J = 5.4 Hz, 1H), 5.10-5.00 (m, 1H), 4.78-4.67 (m, 2H), 4.11 (t, J = 5.5 Hz, 2H), 3.89 (t, J = 5.4 Hz, 2H), 2.58 (s, 3H), 2.36 (s, 3H), 1.63 (d, J = 6.7 Hz, 3H). |
| 46 | 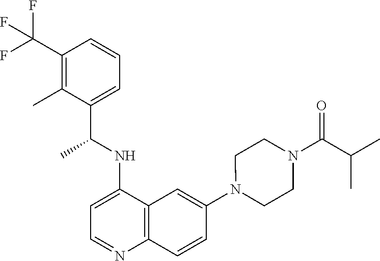 [SMILES: Cc1c([C@@H](C)Nc2ccnc3ccc(N4CCN(C(=O)C(C)C)CC4)cc23)cccc1C(F)(F)F] | FA | LCMS: 485.2 [M + H]⁺. ¹H NMR (400 MHz, DMSO) δ 8.18 (s, 1H), 8.10 (d, J = 5.3 Hz, 1H), 7.69- 7.64 (m, 3H), 7.57 (d, J = 7.6 Hz, 1H), 7.51 (dd, J = 9.3, 2.3 Hz, 1H), 7.38 (d, J = 6.2 Hz, 1H), 7.32 (t, J = 7.8 Hz, 1H), 5.94 (d, J = 5.5 Hz, 1H), 5.09- 5.03 (m, 1H), 3.76-3.68 (m, 4H), 3.36- 3.27 (m, 4H), 3.00-2.94 (m, 1H), 2.57 (s, 3H), 1.61 (d, J = 6.7 Hz, 3H), 1.05 (d, J = 6.7 Hz, 6H). |
| 47 | 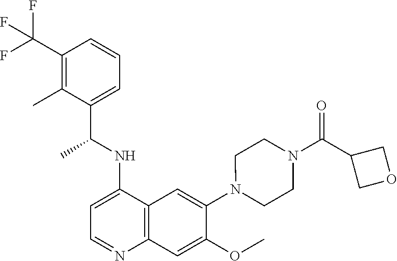 [SMILES: COc1cc2nccc(N[C@H](C)c3cccc(C(F)(F)F)c3C)c2cc1N1CCN(C(=O)C2COC2)CC1] | | LCMS: 529.2 [M + H]⁺. ¹H NMR (400 MHz, DMSO) δ 8.14 (d, J = 5.6 Hz, 1H), 7.83-7.73 (m, 2H), 7.66 (d, J = 7.6 Hz, 1H), 7.58 (d, J = 7.6 Hz, 1H), 7.33 (t, J = 7.6 Hz, 1H), 7.20 (s, 1H), 5.98 (d, J = 6.0 Hz, 1H), 5.15- 5.07 (m, 1H), 4.76-4.69 (m, 4H), 4.23- 4.15 (m, 1H), 3.92 (s, 3H), 3.73-3.69 (m, 2H), 3.40-3.36 (m, 2H), 3.16- 3.05 (m, 4H), 2.57 (s, 3H), 1.61 (d, J = 6.8 Hz, 3H). |
| 48 | 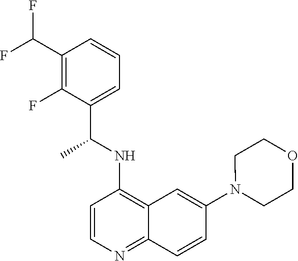 [SMILES: C[C@@H](Nc1ccnc2ccc(N3CCOCC3)cc12)c1cccc(C(F)F)c1F] | | LCMS: 402.2 [M + H]⁺. ¹H NMR (400 MHz, DMSO) δ 8.35 (t, J = 6.0 Hz, 1H), 7.92-7.84 (m, 2H), 7.83- 7.78 (m, 1H), 7.76-7.69 (m, 1H), 7.60 (t, J = 6.8 Hz, 1H), 7.41-7.24 (m, 2H), 6.54 (d, J = 6.4 Hz, 1H), 5.42 (t, J = 6.8 Hz, 1H), 3.82 (d, J = 4.8 Hz, 3H), 3.71-3.52 (m, 2H), 3.44-3.36 (m, 4H), 1.77 (d, J = 6.8 Hz, 3H). |
| 49 | 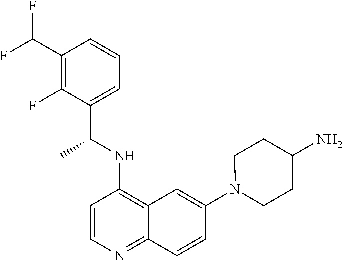 [SMILES: C[C@@H](Nc1ccnc2ccc(N3CCC(N)CC3)cc12)c1cccc(C(F)F)c1F] | HCl | LC/MS: 415.2 [M + H]⁺. ¹H NMR (400 MHz, DMSO) δ 8.46 (s, 1H), 8.08 (d, J = 5.2 Hz, 1H), 7.63 (d, J = 9.2 Hz, 1H), 7.61-7.57 (m, 2H), 7.51 (t, J = 6.9 Hz, 1H), 7.48-7.43 (m, 1H), 7.40-7.14 (m, 3H), 6.05 (d, J = 5.3 Hz, 1H), 5.10-5.03 (m, 1H), 3.90 (d, J = 9.9 Hz, 2H), 3.05-3.01 (m, 1H), 2.89- 2.83 (m, 2H), 1.99-1.93 (m, 2H), 1.67 (d, J = 6.7 Hz, 3H), 1.62-1.52 (m, 2H). |
| 50 | 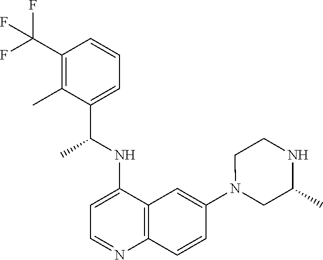 [SMILES: Cc1c([C@@H](C)Nc2ccnc3ccc(N4CCN[C@H](C)C4)cc23)cccc1C(F)(F)F] | FA | LC/MS: 429.2 [M + H]⁺. ¹H NMR (400 MHz, DMSO) δ 8.30 (brs, 1H), 8.09 (d, J = 4.8 Hz, 1H), 7.73- 7.59 (m, 3H), 7.57 (d, J = 7.6 Hz, 1H), 7.50 (d, J = 9.2 Hz, 1H), 7.40-7.26 (m, 2H), 5.92 (d, J = 5.2 Hz, 1H), 5.08- 5.01 (m, 1H), 3.92-3.78 (m, 2H), 3.35- 2.79 (m, 5H), 2.67-2.52 (m, 4H), 1.61 (d, J = 6.4 Hz, 3H), 1.22 (d, J = 6.4 Hz, 3H). |
| 51 | 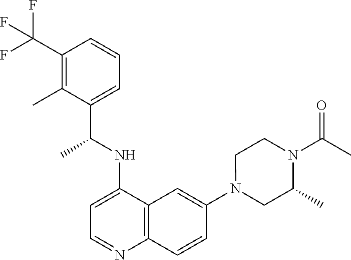 [SMILES: CC(=O)N1CCN(c2ccc3nccc(N[C@H](C)c4cccc(C(F)(F)F)c4C)c3c2)C[C@H]1C] | FA | LC/MS: 471.3 [M + H]⁺. ¹H NMR (400 MHz, DMSO) δ 8.22 (s, 1H), 8.11 (d, J = 5.6 Hz, 1H), 7.72- 7.60 (m, 3H), 7.57 (d, J = 7.6 Hz, 1H), 7.54-7.47 (m, 2H), 7.32 (t, J = 7.6 Hz, 1H), 5.97 (d, J = 5.6 Hz, 1H), 5.13- 5.03 (m, 1H), 4.42-4.19 (m, 1H), 3.97- 3.63 (m, 3H), 3.54 (t, J = 12.0 Hz, 1H), 3.13-2.81 (m, 2H), 2.58 (s, 3H), 2.08 (d, J = 12.4 Hz, 3H), 1.62 (d, J = 6.4 Hz, 3H), 1.39-1.20 (m, 3H). |
| 52 | 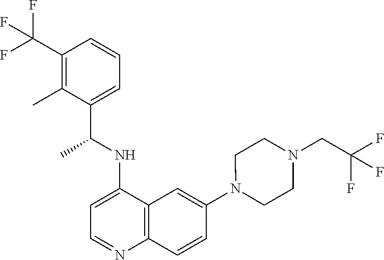 [SMILES: Cc1c([C@@H](C)Nc2ccnc3ccc(N4CCN(CC(F)(F)F)CC4)cc23)cccc1C(F)(F)F] | FA | LC/MS: 497.2 [M + H]⁺. ¹H NMR (400 MHz, DMSO) δ 8.36 (brs, 2H), 8.09-8.04 (m, 1H), 7.69- 7.60 (m, 3H), 7.56 (d, J = 7.3 Hz, 1H), 7.45 (dd, J = 9.2, 2.4 Hz, 1H), 7.31 (t, J = 7.8 Hz, 1H), 7.22 (d, J = 6.5 Hz, 1H), 5.90 (d, J = 5.4 Hz, 1H), 5.06-4.99 (m, 1H), 3.39-3.24 (m, 6H), 2.92-2.87 (m, 4H), 2.57 (s, 3H), 1.60 (d, J = 6.7 Hz, 3H). |
| 53 | 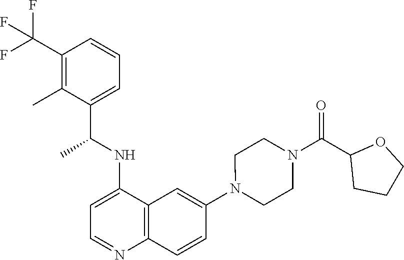 [SMILES: Cc1c([C@@H](C)Nc2ccnc3ccc(N4CCN(C(=O)C5CCCO5)CC4)cc23)cccc1C(F)(F)F] | FA | LC/MS: 513.2 [M + H]⁺. ¹H NMR (400 MHz, DMSO) δ 8.30 (s, 1H), 8.08 (d, J = 5.2 Hz, 1H), 7.70- 7.62 (m, 3H), 7.56 (d, J = 7.6 Hz, 1H), 7.49 (dd, J = 9.2, 2.4 Hz, 1H), 7.35- 7.25 (m, 2H), 5.91 (d, J = 5.2 Hz, 1H), 5.07-5.00 (m, 1H), 4.76 (dd, J = 7.6, 5.6 Hz, 1H), 3.85-3.70 (m, 5H), 3.40- 3.15 (m, 5H), 2.57 (s, 3H), 2.13-2.01 (m, 2H), 1.90-1.80 (m, 2H), 1.60 (d, J = 6.7 Hz, 3H). |
| 54 | 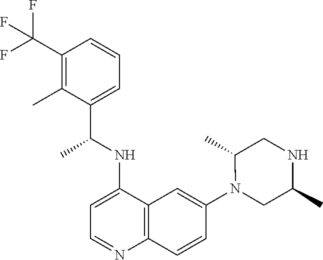 [SMILES: Cc1c([C@@H](C)Nc2ccnc3ccc(N4C[C@H](C)NC[C@H]4C)cc23)cccc1C(F)(F)F] | HCl | LC/MS: 443.2 [M + H]⁺. ¹H NMR (400 MHz, DMSO) δ 9.78 (brs, 1H), 9.37 (s, 1H), 8.40-8.26 (m, 2H), 7.96 (d, J = 9.2 Hz, 1H), 7.80- 7.74 (m, 2H), 7.63 (d, J = 7.8 Hz, 1H), 7.39 (t, J = 7.8 Hz, 1H), 6.29 (d, J = 7.0 Hz, 1H), 5.37-5.28 (m, 1H), 4.20- 4.05 (m, 1H), 3.65-3.55 (m, 1H), 3.54- 3.48 (m, 1H), 3.47-3.37 (m, 2H), 3.01-2.94 (m, 1H), 2.58 (s, 3H), 1.72 (d, J = 6.6 Hz, 3H), 1.37 (d, J = 6.6 Hz, 3H), 1.13 (d, J = 6.4 Hz, 3H). |
| 55 | 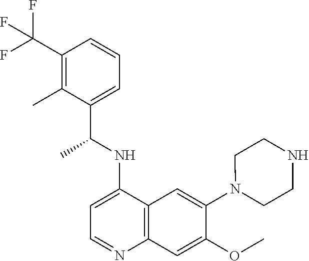 [SMILES: COc1cc2nccc(N[C@H](C)c3cccc(C(F)(F)F)c3C)c2cc1N1CCNCC1] | | LCMS: 445.1 [M + H]⁺ ¹H NMR (400 MHz, DMSO) δ 8.32- 8.27 (m, 2H), 8.09 (d, J = 5.2 Hz, 1H), 7.75-7.70 (m, 1H), 7.66 (d, J = 7.6 Hz, 1H), 7.57 (d, J = 7.6 Hz, 1H), 7.32 (t, J = 7.6 Hz, 1H), 7.20-7.15 (m, 1H), 5.91- 5.84 (m, 1H), 5.07-4.99 (m, 1H), 3.90 (s, 3H), 3.27-3.14 (m, 8H), 2.57 (s, 3H), 1.60 (d, J = 6.8 Hz, 3H). |
| 56 | 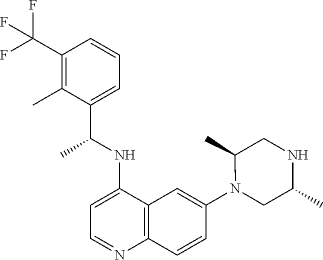 [SMILES: Cc1c([C@@H](C)Nc2ccnc3ccc(N4C[C@@H](C)NC[C@@H]4C)cc23)cccc1C(F)(F)F] | HCl | LC/MS: 443.2 [M + H]⁺. ¹H NMR (400 MHz, DMSO) δ 9.81 (brs, 1H), 9.37 (s, 1H), 8.38-8.29 (m, 2H), 7.96 (d, J = 9.2 Hz, 1H), 7.81- 7.76 (m, 2H), 7.62 (d, J = 7.8 Hz, 1H), 7.38 (t, J = 7.8 Hz, 1H), 6.28 (d, J = 6.8 Hz, 1H), 5.34-5.27 (m, 1H), 4.10- 4.00 (m, 1H), 3.65-3.55 (m, 1H), 3.55- 3.45 (m, 1H), 3.45-3.38 (m, 2H), 2.96 (dd, J = 12.8, 6.9 Hz, 1H), 2.58 (s, 3H), 1.73 (d, J = 6.6 Hz, 3H), 1.37 (d, J = 6.6 Hz, 3H), 1.11 (d, J = 6.3 Hz, 3H). |
| 57 | 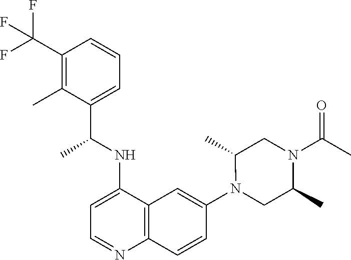 [SMILES: CC(=O)N1C[C@@H](C)N(c2ccc3nccc(N[C@H](C)c4cccc(C(F)(F)F)c4C)c3c2)C[C@@H]1C] | | LC/MS: 485.2 [M + H]⁺. ¹H NMR (400 MHz, DMSO) δ 8.06 (d, J = 5.2 Hz, 1H), 7.70-7.63 (m, 2H), 7.59- 7.52 (m, 2H), 7.50-7.43 (m, 1H), 7.32 (t, J = 7.8 Hz, 1H), 7.22 (d, J = 5.6 Hz, 1H), 5.92-5.86 (m, 1H), 5.10- 4.80 (m, 2H), 4.40-4.20 (m, 2H), 3.76- 3.62 (m, 1H), 3.48-3.41 (m, 1H), 3.25-3.17 (m, 1H), 2.57 (s, 3H), 2.15 (s, 1.5H), 2.05 (s, 1.5 H), 1.61 (d, J = 6.7 Hz, 3H), 1.35-1.22 (m, 3H), 0.98- 0.85 (m, 3H). |
| 58 | 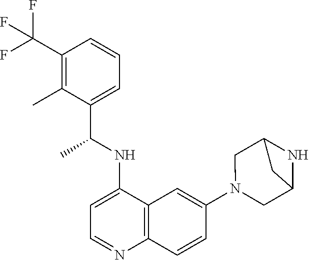 [SMILES: Cc1c([C@@H](C)Nc2ccnc3ccc(N4CC5CC(C4)N5)cc23)cccc1C(F)(F)F] | FA | LC/MS: 427.2 [M + H]⁺. ¹H NMR (400 MHz, DMSO) δ 8.27 (s, 2H), 8.07-8.04 (m, 1H), 7.73 (d, J = 9.0 Hz, 1H), 7.67-7.62 (m, 1H), 7.57 (d, J = 7.7 Hz, 1H), 7.45-7.25 (m, 4H), 7.21-7.05 (m, 1H), 5.96-5.92 (m, 1H), 5.11-5.04 (m, 1H), 4.47-4.43 (m, 0.5H), 4.23 (d, J = 4.9 Hz, 1H), 3.91-3.72 (m, 3H), 3.62-3.52 (m, 0.5H), 3.37-3.09 (m, 1H), 2.82-2.76 (m, 1H), 2.58 (s, 3H), 1.78 (d, J = 9.2 Hz, 1H), 1.63-1.59 (m, 3H). |
| 59 | 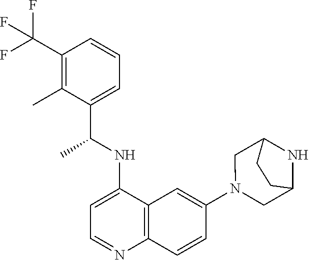 | HCl | LC/MS: 429.2 [M + H]⁺. ¹H NMR (400 MHz, DMSO) δ 8.26 (s, 1H), 8.07 (d, J = 5.3 Hz, 1H), 7.68- 7.63 (m, 2H), 7.57 (d, J = 7.7 Hz, 1H), 7.51 (d, J = 2.2 Hz, 1H), 7.44 (dd, J = 9.3, 2.4 Hz, 1H), 7.35-7.28 (m, 2H), 5.91 (d, J = 5.5 Hz, 1H), 5.08-5.02 (m, 1H), 3.96 (s, 2H), 3.82-3.73 (m, 2H), 3.13-3.05 (m, 2H), 2.57 (s, 3H), 1.92 (s, 4H), 1.61 (d, J = 6.7 Hz, 3H). |
| 60 | 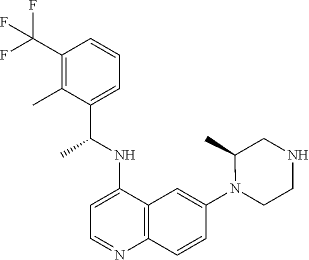 [SMILES: Cc1c([C@@H](C)Nc2ccnc3ccc(N4CCNC[C@@H]4C)cc23)cccc1C(F)(F)F] | FA | LC/MS: 429.2 [M + H]⁺. ¹H NMR (400 MHz, DMSO) δ 8.29 (s, 2H), 8.09 (d, J = 4.8 Hz, 1H), 7.71- 7.64 (m, 2H), 7.62 (s, 1H), 7.57 (d, J = 7.6 Hz, 1H), 7.48 (d, J = 9.2 Hz, 1H), 7.32 (t, J = 7.6 Hz, 1H), 5.97-5.91 (m, 1H), 5.08-5.02 (m, 1H), 4.24-4.17 (m, 1H), 3.54-3.45 (m, 1H), 3.35- 3.10 (m, 3H), 3.06-2.89 (m, 2H), 2.57 (s, 3H), 1.61 (d, J = 6.8 Hz, 3H), 1.05 (d, J = 5.6 Hz, 3H). |
| 61 | 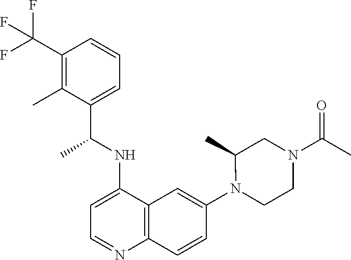 [SMILES: CC(=O)N1CCN(c2ccc3nccc(N[C@H](C)c4cccc(C(F)(F)F)c4C)c3c2)[C@@H](C)C1] | | LC/MS: 471.1 [M + H]⁺. ¹H NMR (400 MHz, DMSO) δ 8.59 (brs, 1H), 8.25 (d, J = 6.4 Hz, 1H), 7.82- 7.71 (m, 3H), 7.67 (d, J = 7.6 Hz, 1H), 7.62 (d, J = 7.6 Hz, 1H), 7.37 (t, J = 7.6 Hz, 1H), 6.22 (dd, J = 6.4, 2.4 Hz, 1H), 5.33-5.21 (m, 1H), 4.47-4.19 (m, 2H), 3.99-3.75 (m, 1H), 3.67-3.58 (m, 1H), 3.25-2.86 (m, 3H), 2.58 (s, 3H), 2.10 (d, J = 26.4 Hz, 3H), 1.68 (d, J = 6.8 Hz, 3H), 0.97 (dd, J = 17.2, 8.8 Hz, 3H). |
| 62 | 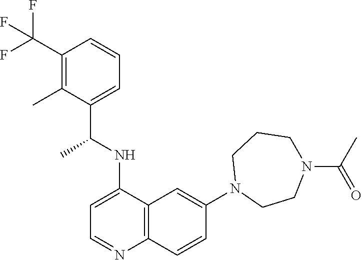 [SMILES: CC(=O)N1CCCN(c2ccc3nccc(N[C@H](C)c4cccc(C(F)(F)F)c4C)c3c2)CC1] | | LC/MS: 471.1 [M + H]⁺. ¹H NMR (400 MHz, DMSO) δ 8.10 (d, J = 5.6 Hz, 1H), 7.94 (brs, 1H), 7.72- 7.64 (m, 2H), 7.59 (d, J = 7.6 Hz, 1H), 7.46 (d, J = 8.0 Hz, 2H), 7.35 (t, J = 7.6 Hz, 1H), 6.05 (d, J = 5.6 Hz, 1H), 5.22- 5.12 (m, 1H), 3.95-3.85 (m, 1H), 3.81- 3.68 (m, 4H), 3.45-3.37 (m, 3H), 2.57 (s, 3H), 2.04-1.90 (m, 3H), 1.87- 1.82 (m, 2H), 1.65 (d, J = 6.4 Hz, 3H). |
| 63 | 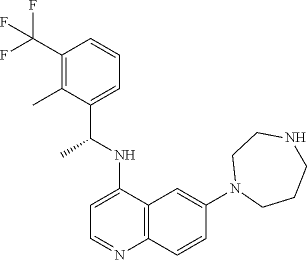 [SMILES: Cc1c([C@@H](C)Nc2ccnc3ccc(N4CCCNCC4)cc23)cccc1C(F)(F)F] | TFA | LC/MS: 429.3 [M + H]⁺. ¹H NMR (400 MHz, DMSO) δ 8.99 (d, J = 5.6 Hz, 2H), 8.27 (d, J = 4.8 Hz, 1H), 7.81 (d, J = 9.2 Hz, 1H), 7.73-7.59 (m, 4H), 7.38 (t, J = 7.6 Hz, 1H), 6.29 (d, J = 6.8 Hz, 1H), 5.40-5.29 (m, 1H), 3.96- 3.92 (m, 2H), 3.73 (t, J = 5.6 Hz, 2H), 3.38-3.34 (m, 2H), 3.23-3.18 (m, 2H), 2.58 (s, 3H), 2.22-2.11 (m, 2H), 1.69 (d, J = 6.4 Hz, 3H). |
| 64 | 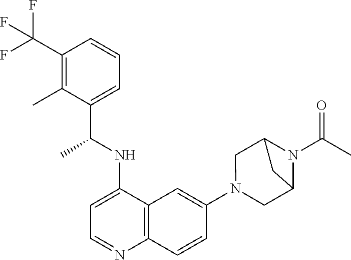 [SMILES: CC(=O)N1C2CC1CN(c1ccc3nccc(N[C@H](C)c4cccc(C(F)(F)F)c4C)c3c1)C2] | FA | LC/MS: 469.2 [M + H]⁺. ¹H NMR (400 MHz, DMSO) δ 8.27 (s, 1H), 8.01 (d, J = 5,2 Hz, 1H), 7.68- 7.64 (m, 2H), 7.55 (d, J = 7.9 Hz, 1H), 7.32-7.27 (m, 3H), 7.19-7.13 (m, 1H), 5.92-5.86 (m, 1H), 5.08-5.00 (m, 1H), 4.74-4.70 (m, 1H), 4.50- 4.47 (m, 1H), 4.04-3.92 (m, 1H), 3.81- 3.68 (m, 2H), 3.57-3.47 (m, 1H), 2.70-2.68 (m, 1H), 2.58 (s, 3H), 1.87 (d, J = 3.7 Hz, 3H), 1.66 (d, J = 8.5 Hz, 1H), 1.61 (d, J = 6.6 Hz, 3H). |
| 65 | 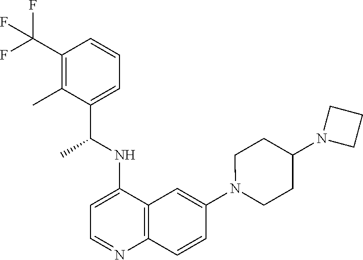 [SMILES: Cc1c([C@@H](C)Nc2ccnc3ccc(N4CCC(N5CCC5)CC4)cc23)cccc1C(F)(F)F] | FA | LC/MS: 469.2 [M + H]⁺. ¹H NMR (400 MHz, MeOD) δ 8.53 (brs, 1H), 8.14 (d, J = 6.8 Hz, 1H), 7.84 (s, 1H), 7.80-7.70 (m, 2H), 7.69-7.59 (m, 2H), 7.34 (t, J = 7.9 Hz, 1H), 6.32 (d, J = 6.9 Hz, 1H), 5.39-5.31 (m, 1H), 4.21-4.10 (m, 2H), 3.98 (t, J = 7.8 Hz, 4H), 3.20-3.10 (m, 1H), 3.01 (t, J = 12.6 Hz, 2H), 2.63 (s, 3H), 2.47-2.36 (m, 2H), 2.20-2.05 (m, 2H), 1.78 (d, J = 6.8 Hz, 3H), 1.65-1.50 (m, 2H). |
| 66 | 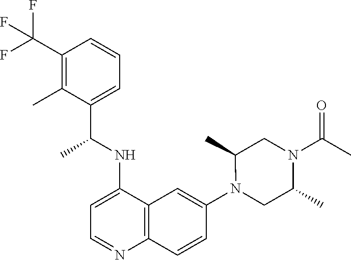 [SMILES: CC(=O)N1C[C@H](C)N(c2ccc3nccc(N[C@H](C)c4cccc(C(F)(F)F)c4C)c3c2)C[C@H]1C] | | LC/MS: 485.2 [M + H]⁺. ¹H NMR (400 MHz, DMSO) δ 8.93 (d, J = 3.2 Hz, 1H), 8.30 (d, J = 6.8 Hz, 1H), 7.87-7.73 (m, 3H), 7.69-7.62 (m, 2H), 7.40 (t, J = 7.8 Hz, 1H), 6.29 (d, J = 6.9 Hz, 1H), 5.38-5.31 (m, 1H), 4.89- 4.25 (m, 3H), 3.74-3.53 (m, 2H), 3.26-3.16 (m, 1H), 2.58 (s, 3H), 2.20- 2.00 (m, 3H), 1.70 (d, J = 6.7 Hz, 3H), 1.24 (dd, J = 26.9, 18.0 Hz, 3H), 0.98 (dd, J = 29.8, 6.5 Hz, 3H). |
| 67 | 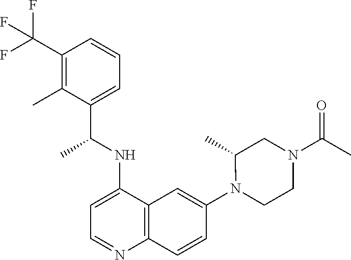 [SMILES: CC(=O)N1CCN(c2ccc3nccc(N[C@H](C)c4cccc(C(F)(F)F)c4C)c3c2)[C@H](C)C1] | | LCMS: 471.2 [M + H]⁺. ¹H NMR (400 MHz, DMSO) δ 8.93 (d, J = 5.6 Hz, 1H), 8.29 (d, J = 6.4 Hz, 1H), 7.86-7.73 (m, 3H), 7.70-7.61 (m, 2H), 7.44-7.36 (m, 1H), 6.35-6.27 (m, 1H), 5.39-5.32 (m, 1H), 4.42- 4.36 (m, 1H), 4.01-3.91 (m, 1H), 3.84- 3.76 (m, 1H), 3.70-3.56 (m, 2H), 3.15-3.10 (m, 1H), 3.00-2.91 (m, 1H), 2.57 (s, 3H), 2.51 (s, 3H), 2.10 (d, J = 23.3 Hz, 3H), 1.70 (d, J = 6.8 Hz, 3H). |
| 68 | 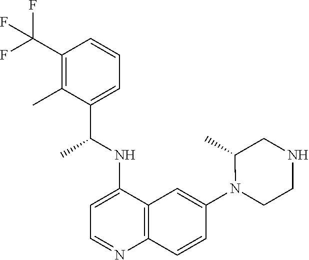 [SMILES: Cc1c([C@@H](C)Nc2ccnc3ccc(N4CCNC[C@H]4C)cc23)cccc1C(F)(F)F] | FA | LCMS: 429.1 [M + H]⁺. ¹H NMR (400 MHz, DMSO) δ 8.32 (d, J = 6.8 Hz, 1H), 7.97 (s, 1H), 7.88-7.84 (m, 1H), 7.82-7.77 (m, 1H), 7.71 (d, J = 7.6 Hz, 1H), 7.64 (d, J = 7.2 Hz, 1H), 7.42-7.37 (m, 1H), 7.26 (s, 1H), 7.13 (s, 1H), 7.01 (s, 1H), 6.31 (d, J = 7.6 Hz, 1H), 5.36-5.32 (m, 1H), 4.58- 4.53 (m, 1H), 3.86-3.75 (m, 2H), 3.45- 3.34 (m, 4H), 2.58 (s, 3H), 1.71 (d, J = 6.8 Hz, 3H), 1.20-1.17 (m, 3H). |
| 69 | 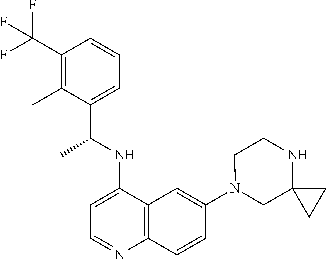 [SMILES: Cc1c([C@@H](C)Nc2ccnc3ccc(N4CCNC5(CC5)C4)cc23)cccc1C(F)(F)F] | | LCMS: 441.2[M + H]⁺. ¹H NMR (400 MHz, DMSO) δ 8.19 (d, J = 6.2 Hz, 1H), 7.72-7.59 (m, 5H), 7.36 (t, J = 7.7 Hz, 1H), 6.12 (d, J = 7.4 Hz, 1H), 5.27-5.16 (m, 1H), 3.52-3.46 (m, 2H), 3.28-3.25 (m, 2H), 3.14- 3.09 (m, 2H), 2.57 (s, 3H), 1.66 (d, J = 6.7 Hz, 3H), 0.82-0.67 (m, 4H). |
| 70 | 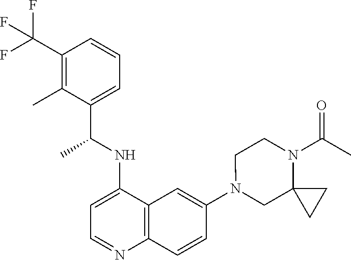 [SMILES: CC(=O)N1CCN(c2ccc3nccc(N[C@H](C)c4cccc(C(F)(F)F)c4C)c3c2)CC12CC2] | | LCMS: 483.2[M + H]⁺. ¹H NMR (400 MHz, DMSO) δ 8.89 (brs, 1H), 8.28 (d, J = 6.8 Hz, 1H), 7.84- 7.76 (m, 3H), 7.69 (d, J = 7.7 Hz, 1H), 7.63 (d, J = 7.6 Hz, 1H), 7.38 (t, J = 7.9 Hz, 1H), 6.27 (d, J = 6.9 Hz, 1H), 5.36- 5.30 (m, 1H), 3.87-3.74 (m, 2H), 3.42- 3.37 (m, 1H), 3.31-3.27 (m, 1H), 2.57 (s, 3H), 2.19-2.02 (m, 3H), 1.70 (d, J = 6.7 Hz, 3H), 1.24-1.07 (m, 4H), 1.02-0.80 (m, 2H). |
| 71 | 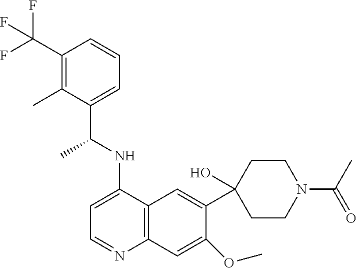 [SMILES: COc1cc2nccc(N[C@H](C)c3cccc(C(F)(F)F)c3C)c2cc1C1(O)CCN(C(C)=O)CC1] | | LCMS: 503.2 [M + H]⁺. ¹H NMR (400 MHz, CDCl₃) δ 9.83 (br, 1H), 8.77 (s, 1H), 8.42 (s, 1H), 7.75 (d, J = 4.0 Hz, 1H), 7.51 (d, J = 4.0 Hz, 1H), 7.28 (br, 1H), 7.22 (br, 1H), 5.98- 5.93 (m, 1H), 4.42 (br, 1H), 3.91 (s, 1H), 3.60 (br, 2H), 3.03 (br, 1H), 2.54 (s, 3H), 2.27 (br, 2H), 2.11 (s, 3H), 1.82 (br, 1H), 1.69 (d, J = 8.0 Hz, 4H). |
| 72 | 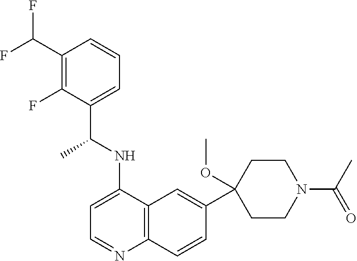 [SMILES: COC1(c2ccc3nccc(N[C@H](C)c4cccc(C(F)F)c4F)c3c2)CCN(C(C)=O)CC1] | | LCMS: 472.1 [M + H]⁺. ¹H NMR (400 MHz, DMSO-d6) δ 9.51 (br, 1H), 8.69 (s, 1H), 8.52 (t, J = 4.0 Hz, 1H), 8.01 (s, 2H), 7.77 (m, 1H), 7.59 (m, 1H), 7.36 (m, 1H), 7.13 (s, 1H), 6.66 (d, J = 8.0 Hz, 1H), 5.49- 5.42 (m, 1H), 4.42 (d, J = 8.0 Hz, 1H), 3.78 (d, J = 8.0 Hz, 1H), 3.40 (t, J = 8.0 Hz, 1H), 2.91 (s, 3H), 2.85 (m, 1H), 2.17 (m, 2H), 2.00 (s, 3H), 1.94 (m, 2H), 1.78 (d, J = 4.0 Hz, 3H). |
| 77 | 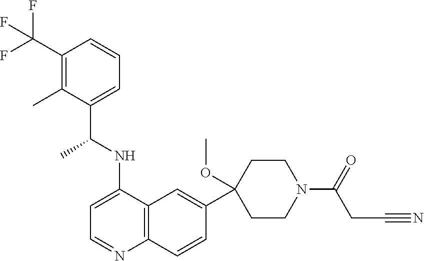 [SMILES: COC1(c2ccc3nccc(N[C@H](C)c4cccc(C(F)(F)F)c4C)c3c2)CCN(C(=O)CC#N)CC1] | | LCMS: 511.0 [M + H]⁺ ¹H NMR (400 MHz, DMSO) δ 9.02 (brs, 1H), 8.60 (s, 1H), 8.42 (d, J = 6.5 Hz, 1H), 7.99-7.89 (m, 2H), 7.68 (t, J = 6.9 Hz, 1H), 7.63 (d, J = 7.8 Hz, 1H), 7.39 (t, J = 7.5 Hz, 1H), 6.32 (dd, J = 6.2, 3.2 Hz, 1H), 5.35-5.26 (m, 1H), 4.40 (d, J = 11.7 Hz, 1H), 4.12-4.10 (m, 2H), 3.64 (d, J = 13.1 Hz, 1H), 3.42 (t, J = 12.9 Hz, 1H), 3.02-2.92 (m, 4H), 2.58 (s, 3H), 2.28-2.20 (m, 1H), 2.14-2.06 (m, 2H), 1.99-1.91 (m, 1H), 1.69 (d, J = 6.6 Hz, 3H). |
| 78 | 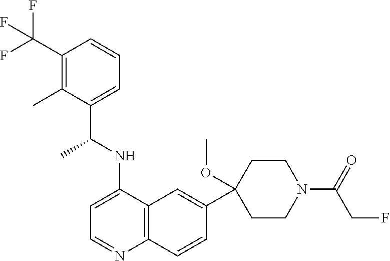 [SMILES: COC1(c2ccc3nccc(N[C@H](C)c4cccc(C(F)(F)F)c4C)c3c2)CCN(C(=O)CF)CC1] | | LCMS: 504.1 [M + H]⁺ ¹H NMR (400 MHz, DMSO) δ 8.49 (s, 1H), 8.44 (brs, 1H), 8.35 (d, J = 6.1 Hz, 1H), 7.89-7.83 (m, 2H), 7.70-7.66 (m, 1H), 7.60 (d, J = 7.8 Hz, 1H), 7.39- 7.32 (m, 1H), 6.18 (d, J = 6.1 Hz, 1H), 5.25 (d, J = 13.3 Hz, 1H), 5.22-5.17 (m, 1H), 5.14 (d, J = 13.3 Hz, 1H), 4.39 (d, J = 11.8 Hz, 1H), 3.52 (d, J = 12.3 Hz, 1H), 3.41-3.37 (m, 1H), 3.00- 2.92 (m, 4H), 2.58 (s, 3H), 2.26-2.20 (m, 1H), 2.15-2.04 (m, 2H), 2.02- 1.95 (m, 1H), 1.66 (d, J = 6.7 Hz, 3H). |
| 79 | 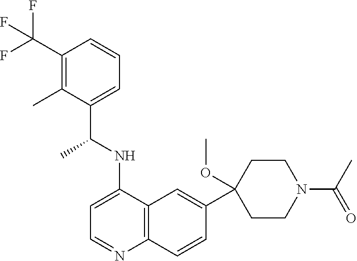 | | LCMS: 625.1 [M + H]⁺ ¹H NMR (400 MHz, DMSO) δ 8.32 (brs, 1H), 8.21 (brs, 1H), 8.11 (brs, 1H), 7.82 (d, J = 8.6 Hz, 1H), 7.66- 7.56 (m, 2H), 7.25-7.18 (m, 1H), 6.14- 6.04 (m, 1H), 5.15-5.00 (m, 1H), 4.70-4.55 (m, 1H), 3.81-3.69 (m, 1H), 3.65-3.50 (m, 1H), 3.10-2.92 (m, 4H), 2.59 (s, 3H), 2.28-1.86 (m, 8H), 1.76 (d, J = 6.2 Hz, 3H). |
| 80 | 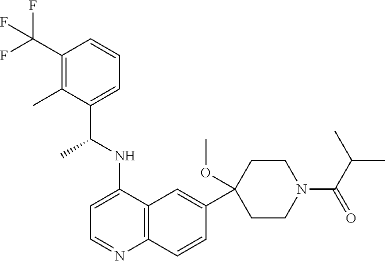 [SMILES: COC1(c2ccc3nccc(N[C@H](C)c4cccc(C(F)(F)F)c4C)c3c2)CCN(C(=O)C(C)C)CC1] | FA | LCMS: 514.3 [M + H]⁺ ¹H NMR (400 MHz, DMSO) δ 8.33 (brs, 1H), 8.24 (d, J = 5.3 Hz, 1H), 8.20 (brs, 1H), 7.78 (d, J = 8.8 Hz, 1H), 7.73- 7.54 (m, 4H), 7.32 (t, J = 7.8 Hz, 1H), 5.98 (d, J = 4.8 Hz, 1H), 5.08-4.99 (m, 1H), 4.47-4.45 (m, 1H), 3.91 (d, J = 12.8 Hz, 1H), 3.47-3.41 (m, 1H), 2.96- 2.93 (m, 5H), 2.58 (s, 3H), 2.25-2.17 (m, 2H), 2.10-1.88 (m, 2H), 1.61 (d, J = 6.7 Hz, 3H), 1.02 (d, J = 6.7 Hz, 6H). |
| 81 | 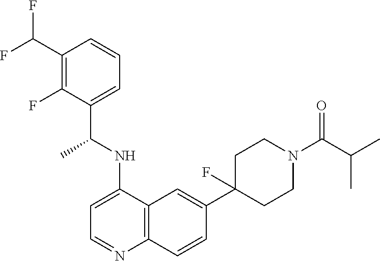 [SMILES: CC(C)C(=O)N1CCC(F)(c2ccc3nccc(N[C@H](C)c4cccc(C(F)F)c4F)c3c2)CC1] | TFA | LCMS: 488.3 [M + H]⁺ ¹H NMR (400 MHz, DMSO) δ 14.07 (s, 1H), 9.40 (d, J = 6.8 Hz, 1H), 8.76 (s, 1H), 8.56 (d, J = 7.0 Hz, 1H), 8.13- 8.06 (m, 1H), 7.94 (d, J = 8.9 Hz, 1H), 7.70 (t, J = 7.3 Hz, 1H), 7.61 (t, J = 6.9 Hz, 1H), 7.38 (d, J = 8.4 Hz, 1H), 7.24 (t, J = 45.7 Hz, 1H), 6.70 (d, J = 7.1 Hz, 1H), 5.52-5.42 (m, 1H), 4.60 (d, J = 12.2 Hz, 1H), 4.08 (d, J = 12.1 Hz, 1H), 3.47-3.38 (m, 1H), 3.02-2.87 (m, 2H), 2.16-2.08 (m, 4H), 1.76 (d, J = 6.7 Hz, 3H), 1.09-0.99 (m, 6H). |
| 82 | 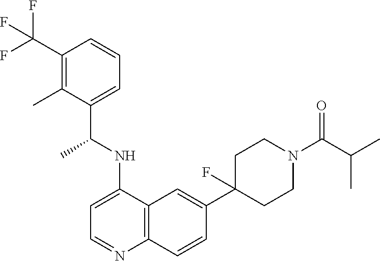 [SMILES: Cc1c([C@@H](C)Nc2ccnc3ccc(C4(F)CCN(C(=O)C(C)C)CC4)cc23)cccc1C(F)(F)F] | TFA | LCMS: 502.3 [M + H]⁺ ¹H NMR (400 MHz, DMSO) δ 14.03 (s, 1H), 9.42 (d, J = 6.4 Hz, 1H), 8.80 (s, 1H), 8.50 (d, J = 7.1 Hz, 1H), 8.13- 8.07 (m, 1H), 7.93 (d, J = 8.9 Hz, 1H), 7.70-7.64 (m, 2H), 7.40 (t, J = 7.8 Hz, 1H), 6.45 (d, J = 7.1 Hz, 1H), 5.43- 5.35 (m, 1H), 4.64-4.54 (m, 1H), 4.13- 4.04 (m, 1H), 3.47-3.40 (m, 1H), 3.04-2.90 (m, 2H), 2.57 (s, 3H), 2.23- 2.10 (m, 4H), 1.70 (d, J = 6.7 Hz, 3H), 1.09-0.99 (m, 6H). |
| 83 | 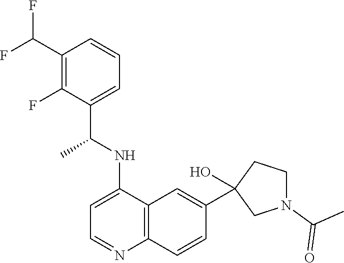 [SMILES: CC(=O)N1CCC(O)(c2ccc3nccc(N[C@H](C)c4cccc(C(F)F)c4F)c3c2)C1] | | LCMS: 444.2 [M + H]⁺ ¹H NMR (400 MHz, DMSO) δ 8.52 (d, J = 18.4 Hz, 1H), 8.28 (d, J = 5.3 Hz, 1H), 7.80-7.77 (m, 1H), 7.78 (dd, J = 8.8, 3.0 Hz, 1H), 7.71-7.58 (m, 2H), 7.53 (t, J = 7.0 Hz, 1H), 7.43-7.11 (m, 2H), 6.17 (d, J = 5.4 Hz, 1H), 5.69- 5.57 (m, 1H), 5.11 (t, J = 6.8 Hz, 1H), 3.90-3.80 (m, 1H), 3.78-3.64 (m, 2H), 3.60-3.45 (m, 1H), 2.47-2.37 (m, 1H), 2.31-2.14 (m, 1H), 2.05-1.95 (m, 3H), 1.69 (d, J = 6.7 Hz, 3H). |
| 84 | 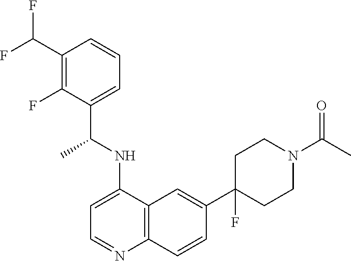 [SMILES: CC(=O)N1CCC(F)(c2ccc3nccc(N[C@H](C)c4cccc(C(F)F)c4F)c3c2)CC1] | | LCMS: 460.1 [M + H]⁺ ¹H NMR (400 MHz, DMSO) δ 8.46 (s, 1H), 8.31 (d, J = 5.4 Hz, 1H), 7.84- 7.80 (m, 1H), 7.79-7.74 (m, 1H), 7.71 (d, J = 6.4 Hz, 1H), 7.60 (t, J = 7.3 Hz, 1H), 7.53 (t, J = 7.1 Hz, 1H), 7.41- 7.12 (m, 2H), 6.20 (d, J = 5.6 Hz, 1H), 5.17-5.07 (m, 1H), 4.56 (d, J = 8.7 Hz, 1H), 3.94 (d, J = 11.1 Hz, 1H), 3.44 (t, J = 11.9 Hz, 1H), 2.92 (t, J = 11.6 Hz, 1H), 2.35-2.13 (m, 2H), 2.11 (s, 3H), 2.00-2.01 (m, 2H), 1.69 (d, J = 6.7 Hz, 3H). |
| 85 | 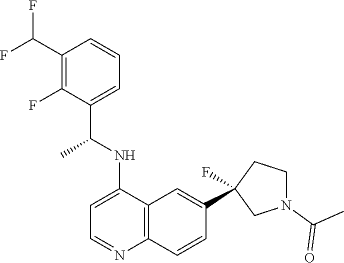 [SMILES: CC(=O)N1CC[C@](F)(c2ccc3nccc(N[C@H](C)c4cccc(C(F)F)c4F)c3c2)C1] | | LCMS: 446.0 [M + H]⁺ ¹H NMR (400 MHz, DMSO) δ 9.14 (brs, 1H), 8.95-8.83 (m, 1H), 8.51 (d, J = 6.6 Hz, 1H), 8.10-8.05 (m, 1H), 7.99-7.95 (m, 1H), 7.75-7.69 (m, 1H), 7.59 (t, J = 6.9 Hz, 1H), 7.41- 7.11 (m, 2H), 6.62-6.58 (m, 1H), 5.43- 5.37 (m, 1H), 4.19-4.12 (m, 1H), 3.92-3.80 (m, 2H), 3.57-3.46 (m, 1H), 2.73-2.60 (m, 2H), 2.08-2.01 (m, 3H), 1.76 (d, J = 6.7 Hz, 3H). |
| 86 | 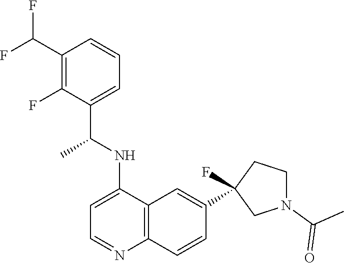 [SMILES: CC(=O)N1CC[C@@](F)(c2ccc3nccc(N[C@H](C)c4cccc(C(F)F)c4F)c3c2)C1] | | LCMS: 446.0 [M + H]⁺ ¹H NMR (400 MHz, DMSO) δ 9.25- 9.08 (m, 1H), 9.02-8.86 (m, 1H), 8.50 (d, J = 6.6 Hz, 1H), 8.03 (dd, J = 23.1, 8.8 Hz, 2H), 7.79-7.70 (m, 1H), 7.59 (t, J = 7.1 Hz, 1H), 7.41-7.12 (m, 2H), 6.57 (d, J = 6.7 Hz, 1H), 5.42-5.34 (m, 1H), 4.21-4.13 (m, 1H), 3.93-3.75 (m, 2H), 3.57-3.45 (m, 1H), 2.80- 2.56 (m, 2H), 2.09-2.01 (m, 3H), 1.80- 1.73 (m, 3H). |

### Testing of Compounds for Inhibition Activity

### Example 22: SOS1/KRAS-G12D Binding Inhibition Assay

SOS1 binding affinity of candidate compounds was measured by monitoring the interaction of SOS1 with KRAS-G12D in the presence of the test compound. Briefly, 5 nM GST tagged SOS1 proteins (final concentration) were pre-incubated with 50 nM 6*His tagged KRAS-G12D proteins (final concentration) in an assay buffer containing 10 mM HEPES, pH 7.4, 150 mM NaCl, 1 mM DTT, 5 mM MgCl₂, 0.1% Tween-20, and 0.05% BSA for 30 minutes. The test compounds in 2% DMSO (final concentration) at various concentrations were then added to the reaction mixture and incubated for another 30 minutes at 4° C. 5 ug/ml GSH AlphaScreen donor beads (PerkinElmer, 6765300) and 5 ug/ml nickel chelate (Ni-NTA) AlphaScreen acceptor beads (PerkinElmer, 6760619C) (final concentrations) were then added to the mixture. After an incubation of 2 hours at 4° C., the fluorescent signals were obtained on the EnVision® 2105 Multilabel Plate Reader (PerkinElmer).

Raw AlphaScreen data were converted to a percentage of inhibition (relative to DMSO) using the following equations:
a) For a given test compound concentration X:

Signal (X)=Signal (SOS1 & KRAS-G12D & compound)-Signal (buffer & KRAS-G12D & compound)

Percentage of inhibition at concentration X=[1−Signal (X)/Signal (DMSO)]*100% b)

The IC50 values were determined by nonlinear regression of plots of [inhibitor] vs. percentage of inhibition with variable slope, analyzed using GraphPad Prism 9.

### Example 23: Phospho-ERK1/2 Expression Inhibition in DLD1 Cells

DLD1 cells were obtained from American Type Culture Collection (ATCC). DLD1 cells were plated in 96-well plates (VWR #10062-900, or Corning #3904) in 90 uL culture medium at a density of 15,000 cells/well in the RPM11640 growth medium containing 10% FBS and 1% Penicillin Streptomycin, and then incubated at 37° C. overnight. The following day, the test compound was administered to the cells by using 1000×x compound stock solution prepared in DMSO at various concentrations. 1000× compound stock solution was first diluted in culturing medium to 10×, then 10 uL compound medium was added to each well in the cell plates. After administration of the compound, the cells were then incubated at 37° C. for 4 hours. Upon completion, the cells were washed with PBS briefly. 150 uL/well of 4% formaldehyde was added and the plates were incubated at room temperature for 20 mins. The cells were washed with PBS briefly, and permeabilized with 150 uL/well of ice cold 100% methanol for 10 mins. The cells were washed with PBS briefly and blocked with 100 uL/well LI-COR blocking buffer for 1 hr at room temperature with gentle shaking. The cells were incubated overnight at 4° C. with 50 uL primary antibody rabbit anti-Phospho-ERK1/2 (1: 500, Cell Signaling, #4370) diluted in Intercept Blocking Buffer (LI-COR, #927-60001) containing 0.1% Tween 20. The next day, the cells were washed with 200 uL PBS containing 0.1% Tween 20, 5×5 mins at room temperature with gentle shaking, and incubated with 50 uL secondary antibody, IRDye® 800CW Goat anti-Rabbit IgG (LI-COR, #926-32211), 1:1000, in LI-COR blocking buffer with 0.2% Tween 20 for 1 hr at room temperature with gentle shaking. The cells were washed with 200 uL PBS containing 0.1% Tween 20, 5×5 mins at room temperature with gentle shaking. The cells were washed with PBS for 5 mins. 100 uL fresh PBS was added to each well and the plates were imaged on a LI-COR Odyssey CLX plate reader.

### Example 24: Cell Growth Inhibition of K562 Cells

K562 cells were obtained from American Type Culture Collection (ATCC). For cell growth assay, K562 cells were seeded in 96-well plates at 1000 cells/well in 90 μl of RPM11640 growth medium containing 10% FBS and 1% Penicillin Streptomycin, and then incubated at 37° C. for 1 hour. The test compound was administered to the cells by using 1000× compound stock solution prepared in DMSO at various concentrations. 1000× compound stock solution was first diluted in culturing medium to 10×, then 10 uL compound medium was added to each well in the cell plates. After administration of the compound, the cells were then incubated at 37° C. for 5 days. Upon completion, the plates were equilibrated at room temperature for approximately 10 minutes. 100 ul of CellTiter-Glo® Reagent (Promega) was added to each well. The plates were then incubated at room temperature for 10 minutes and luminescence was recorded by EnSpire plate reader (PerkinElmer).

TABLE 4
Activities of exemplary compounds

| | KrasG12D/SOS1 | | |
|---|---|---|---|
| | binding IC50 (nM). | DLD1 pERK IC50 | K562 GI50 (nM). |
| | A, ≤10 nM; | (nM). A, ≤150 nM; | A, ≤100 nM; |
| | B, >10 nM | B, >150 nM | B, >100 nM |
| | and ≤100 nM; | and ≤500 nM; | and ≤500 nM; |
| Cpd # | C, >100 nM | C, >500 nM | C, >500 nM |
| 1 | B | C | C |
| 2 | C | N/A | C |
| 3 | C | C | C |
| 4 | B | C | B |
| 5 | B | N/A | C |
| 6 | B | B | B |
| 7 | C | N/A | C |
| 8 | B | B | C |
| 9 | A | B | B |
| 10 | B | C | B |
| 11 | B | B | A |
| 12 | A | A | A |
| 13 | A | B | A |
| 14 | A | B | A |
| 15 | A | A | B |
| 16 | A | A | A |
| 17 | A | A | B |
| 18 | B | C | C |
| 19 | C | C | B |
| 20 | C | C | C |
| 21 | C | C | C |
| 22 | C | C | C |
| 23 | B | C | B |
| 24 | C | C | B |
| 25 | C | B | B |
| 26 | B | C | B |
| 27 | B | C | C |
| 28 | B | C | C |
| 29 | B | C | B |
| 30 | B | C | B |
| 31 | A | C | A |
| 32 | B | C | B |
| 33 | C | C | B |
| 34 | C | C | B |
| 35 | B | C | B |
| 36 | B | C | B |
| 37 | N/A | B | B |
| 38 | C | C | C |
| 39 | C | C | C |
| 40 | A | B | A |
| 41 | A | B | A |
| 42 | A | A | A |
| 43 | A | B | B |
| 44 | A | C | A |
| 45 | B | C | B |
| 46 | B | B | B |
| 47 | B | C | B |
| 48 | A | A | A |
| 49 | A | C | B |
| 50 | B | C | B |
| 51 | B | C | B |
| 52 | C | C | C |
| 53 | B | C | B |
| 54 | B | C | B |
| 55 | B | C | C |
| 56 | B | C | B |
| 57 | C | C | B |
| 58 | B | C | B |
| 59 | B | C | B |
| 60 | B | C | B |
| 61 | C | C | C |
| 62 | C | C | C |
| 63 | B | C | C |
| 64 | B | C | C |
| 65 | B | C | C |
| 66 | C | C | C |
| 67 | N/A | C | C |
| 68 | N/A | C | C |
| 69 | B | C | B |
| 70 | B | C | B |
| 71 | | | |
| 72 | A | A | A |
| 73 | A | C | C |
| 74 | B | B | B |
| 75 | A | C | B |
| 76 | A | A | A |
| 77 | B | B | C |
| 78 | A | B | A |
| 79 | B | B | B |
| 80 | B | C | B |
| 81 | A | A | A |
| 82 | C | C | B |
| 83 | A | B | A |
| 84 | A | B | A |
| 85 | A | C | B |
| 86 | C | N/A | C |
| 87 | C | C | C |
| 88 | B | A | B |
| 89 | A | C | A |
| 90 | B | C | B |
| 91 | A | C | B |
| 92 | A | A | A |
| 93 | N/A | A | A |
| 94 | N/A | A | A |
| 95 | A | C | B |
| 96 | A | B | A |
| 97 | B | C | B |
| 98 | A | C | B |
| 99 | A | B | A |
| 100 | A | B | B |
| 101 | A | C | B |
| 102 | A | A | A |

### Example 25: Study of In Vivo Efficacy in Animal Models

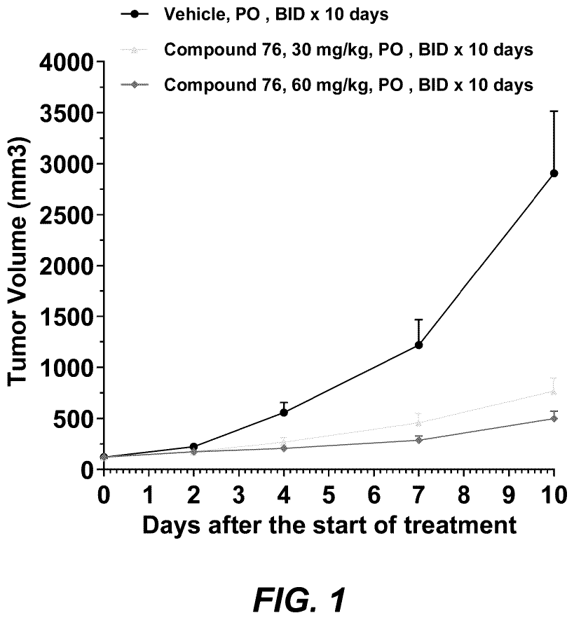

The K562 tumor cells were obtained from ATCC and maintained in vitro as suspension cultured in IMDM medium supplemented with 10% fetal bovine serum and 1% 100 U/mL penicillin and 100 pg/mL streptomycin at 37° C. in an atmosphere of 5% CO₂ in air. The tumor cells were routinely sub-cultured twice weekly. For K562 tumor generation in mice, 5×10⁶ K562 cells in 0.1 mL of PBS mixed with 0.1 mL matrigel (total 0.2 mL) was introduced subcutaneously at the armpit of the right flank of each female NCG mouse. The treatment was started when the average tumor volume reached approximately 120 mm³. The first day of treatment was denoted as Day 0. The test article, Compound 76, was formulated in 10% DMSO/30% PEG400/60% aqueous solution containing 20% HP-β-CD, and then administrated twice daily (BID) for 10 days to the mice according to the predetermined regimen shown in FIG. 1. Tumor volume was determined twice weekly.

### Example 26: Protein Crystallization and Structure Determination

The SOS1 apo crystals were observed after having grown overnight at 18° C. using the sitting-drop vapor diffusion method. The crystallization buffer contains 0.12 M pH 8.5 monosaccharides, 0.1 M Buffer System 3 (Molecular Dimensions), and 50% v/v precipitant Mix2 (Molecular Dimensions). The sitting drop setup was maintained at 18° C. throughout the experiment. Subsequently, the crystals were soaked in a solution containing 0.5 mM Compound 76 for a duration of 17 hours to get the cocrystals of SOS1-Compound 76.

The X-ray data were collected at a temperature of 100 K, using a wavelength of 0.99984 Å, and via the MX300HS detector at TPS 05A. The collected data were subsequently processed, including indexing, integration, and scaling, using the XDS software. The structure was solved through molecular replacement, with the search model being PDB 2110, utilizing the phaser program. Iterative rounds of manual building were performed using Coot, followed by refinement using phenix. refine to generate the final model. The processed data revealed a resolution of 2.0 Å and belonged to the P212121 space group, with the unit cell parameters: a=41.2 Å, b=84.8 Å, c=175.7 Å, α=90°, β=90°, y=90°.

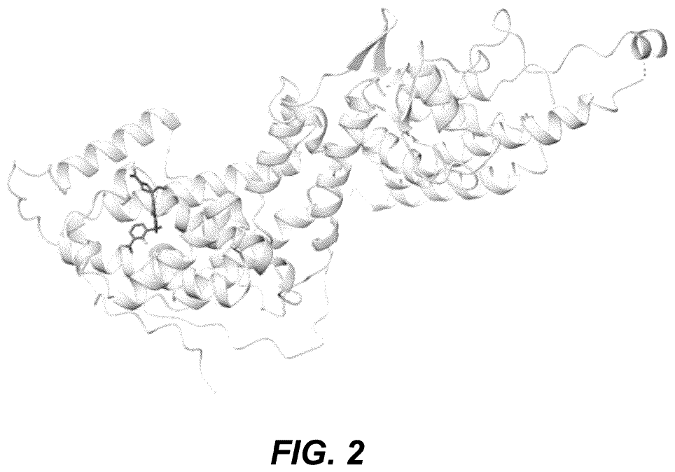

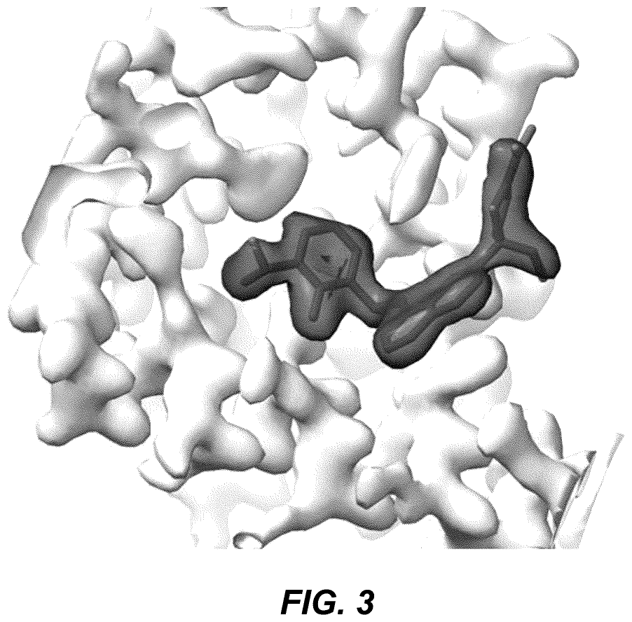

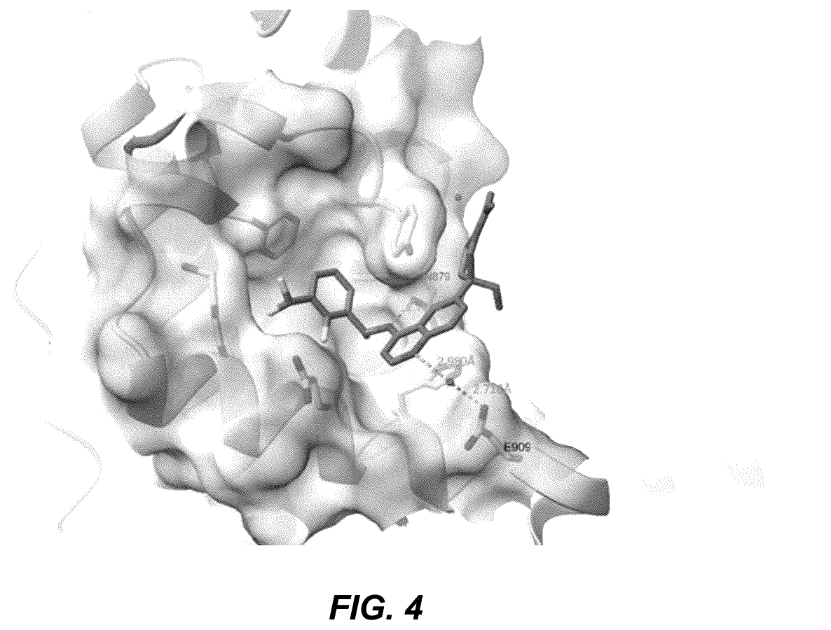

The overall structure of the SOS1-Compound 76 is shown in FIG. 2. Due to the high resolution of the crystal structure, the absolute stereochemistry of Compound 76 was successfully determined using X-ray density analysis, shown in FIG. 3. The interactions between Compound 76 and SOS1 identified by crystal structure are shown in FIG. 4.

What is claimed is:

1. A compound of Formula (I) or a tautomer, stereoisomer or a mixture of stereoisomers, or a pharmaceutically acceptable salt, or hydrate, or deuterated derivative thereof: 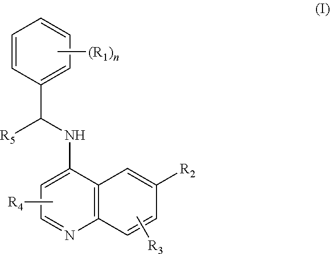
wherein:
R₁ is independently chosen from C₁-C₅ alkyl, halogen, C₁-C₅ alkoxy, C₁-C₄ haloalkyl, and cyano, and n is 0, 1, or 2;
R₂ is chosen from a 4- to 10-membered heteromonocyclic group, fused heterocyclic group, and bridged or spiro heterocyclic group, wherein each of the heteromonocyclic group, fused heterocyclic group, and bridged or spiro heterocyclic group is independently substituted with 0, 1, 2, or 3 R^{a};
R₃ is absent or each R₃ is alkoxyl, C₁-C₅ alkyl, or halo group;
R₄ is absent or each R₄ is cyano or alkyl;
R₅ is a C₁-C₄alkyl;
R^{a} is absent or each R^{a} is independently chosen from a hydrogen, alkoxyl group, deuterated alkoxyl group, deuterated alkyl group, hydroxyl, halo, haloalkyl, alkyl, and 4- to 5-member heterocycle which is independently substituted with 0 or 1 R₆, C(O)R₇, C(O)NR₇, N(R₇)₂, NR₇C(O)R₇, NR₇C(O)OR₇, or C(O)OR₇ group;
R₆ is absent or alkyl; and
each R₇ is independently chosen from hydrogen, alkoxyl, N(CH₃)₂, NHCH₃, a 3- to 6-member cycloalkyl, a 3- to 6-member heterocycle, C₁-C₅ alkyl, CH₂—CN, and haloalkyl.

2. The compound according to claim 1, wherein R₂ is chosen from

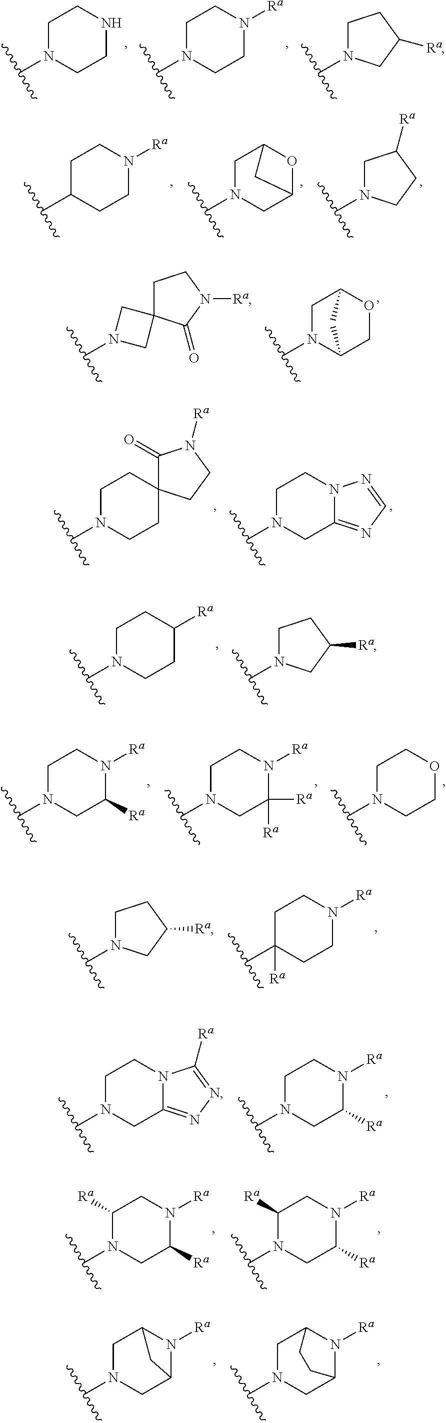

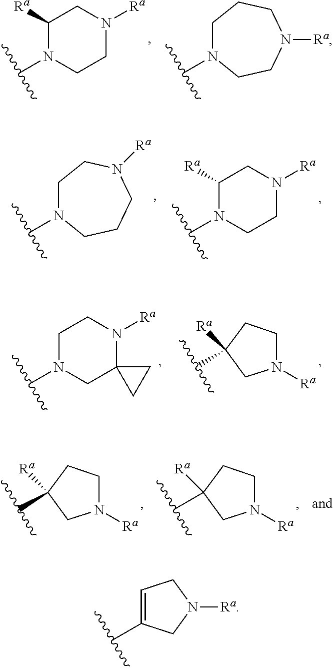

3. The compound according to claim 1, wherein R^{a} is a 4- to 5-member heterocycle which is independently substituted with 0 or 1 R₆.

4. The compound according to claim 3, wherein R^{a} is chosen from

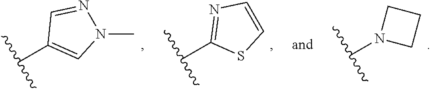

5. The compound according to claim 1, wherein R^{a} is haloalkyl.

6. The compound according to claim 5, wherein R^{a} is F.

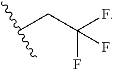 [SMILES: *CC(F)(F)F]

7. The compound according to claim 1, wherein R^{a} is alkyl.

8. The compound according to claim 7, wherein R^{a} is methyl.

9. The compound according to claim 1, wherein R^{a} is alkoxyl.

10. The compound according to claim 9, wherein R^{a} is.

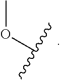 [SMILES: *OC]

11. The compound according to claim 1, wherein R^{a} is halo.

12. The compound according to claim 11, wherein R^{a} is chosen from Cl, Br, and F.

13. The compound according to claim 1, wherein R^{a} is chosen from C(O)R₇, C(O)NR₇, N(R₇)₂, NR₇C(O)R₇, NR₇C(O)OR₇, and C(O)OR₇.

14. The compound according to claim 1, wherein R₆ is C₁-C₃alkyl.

15. The compound according to claim 14, wherein R₆ is chosen from methyl, ethyl, propyl, and isopropyl.

16. The compound according to claim 1, wherein R₇ is a 3- to 5-member cycloalkyl or a 3- to 6-member heterocycle.

17. The compound according to claim 16, wherein R₇ is chosen from

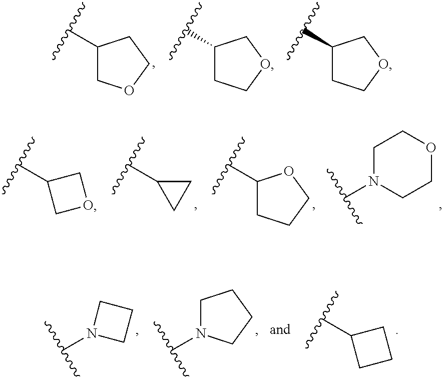

18. The compound according to claim 1, wherein R₇ is hydrogen.

19. The compound according to claim 1, wherein R₇ is hydrogen or alkyl.

20. The compound according to claim 19, wherein R₇ is hydrogen or methyl.

21. The compound according to claim 19, wherein R₇ is hydrogen or

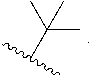 [SMILES: *C(C)(C)C]

22. The compound according to claim 19, wherein R₇ is hydrogen or

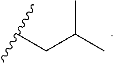 [SMILES: *CC(C)C]

23. The compound according to claim 1, wherein R₇ is alkyl.

24. The compound according to claim 23, wherein R₇ is methyl.

25. The compound according to claim 23, wherein R₇ is

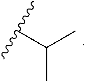 [SMILES: *C(C)C]

26. The compound according to claim 1, wherein R₇ is C₁-C₃ alkyl or

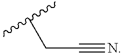 [SMILES: *CC#N]

27. The compound according to claim 26, wherein R₇ is haloalkyl

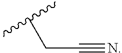 [SMILES: *CC#N]

28. The compound according to claim 1, wherein R₇ is haloalkyl.

29. The compound according to claim 28, wherein R₇ is

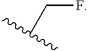 [SMILES: *CF]

30. The compound according to claim 1, wherein R₃ is absent.

31. The compound according to claim 1, wherein R₃ is alkoxyl.

32. The compound according to claim 31, wherein R₃ is

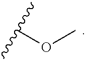 [SMILES: *OC]

33. The compound according to claim 1, wherein R₃ is halo.

34. The compound according to claim 33, wherein R₃ is F.

35. The compound according to claim 1, wherein R₁ is haloalkyl or alkyl.

36. The compound according to claim 35, wherein R₁ is —CF₃ or methyl.

37. The compound according to claim 1, wherein R₁ is haloalkyl or halo.

38. The compound according to claim 37, wherein R₁ is —CHF₂ or F.

39. The compound according to claim 1, wherein R₁ is cyano or alkyl.

40. The compound according to claim 39, wherein R₁ is cyano or methyl.

41. The compound according to claim 1, wherein R₄ is absent.

42. The compound according to claim 1, wherein R₄ is cyano.

43. The compound according to claim 1, wherein R₄ is methyl.

44. The compound according to claim 1, wherein R₅ is a C₁-C₃ alkyl.

45. The compound according to claim 1, wherein R₅ is —CH₃ and a carbon in Formula (I) to which R₅ is directly attached has an (R)- or (S)-configuration.

46. The compound according to claim 45, wherein the carbon in Formula (I) to which R₅ is directly attached has the (R)-configuration.

47. The compound according to claim 1, wherein the compound is chosen from:
(R)-2-methyl-N-(1-(2-methyl-3-(trifluoromethyl)phenyl)ethyl)-6-(piperazin-1-yl)quinolin-4-amine;
(R)-4-((1-(2-methyl-3-(trifluoromethyl)phenyl)ethyl)amino)-6-(piperazin-1-yl)quinoline-3-carbonitrile;
4-(((R)-1-(2-methyl-3-(trifluoromethyl)phenyl)ethyl)amino)-6-(4-(tetrahydrofuran-3-carbonyl)piperazin-1-yl)quinoline-3-carbonitrile;
(4-(2-methyl-4-(((R)-1-(2-methyl-3-(trifluoromethyl)phenyl)ethyl)amino)quinolin-6-yl)piperazin-1-yl)(tetrahydrofuran-3-yl)methanone;
(R)—N-(1-(2-methyl-3-(trifluoromethyl)phenyl)ethyl)-6-(piperazin-1-yl)quinolin-4-amine;
(4-(4-(((R)-1-(2-methyl-3-(trifluoromethyl)phenyl)ethyl)amino)quinolin-6-yl)piperazin-1-yl)(tetrahydrofuran-3-yl)methanone;
N—((R)-1-(3-cyano-4-(((R)-1-(3-(difluoromethyl)-2-fluorophenyl)ethyl)amino)quinolin-6-yl)pyrrolidin-3-yl)acetamide;
tert-butyl ((R)-1-(4-(((R)-1-(3-(difluoromethyl)-2-fluorophenyl)ethyl)amino)quinolin-6-yl)pyrrolidin-3-yl)carbamate;
6-((R)-3-aminopyrrolidin-1-yl)-N—((R)-1-(3-(difluoromethyl)-2-fluorophenyl)ethyl)quinolin-4-amine;
(R)—N-(1-(3-(difluoromethyl)-2-fluorophenyl)ethyl)-6-(piperidin-4-yl)quinolin-4-amine;
N—((R)-1-(4-(((R)-1-(3-(difluoromethyl)-2-fluorophenyl)ethyl)amino)quinolin-6-yl)pyrrolidin-3-yl)acetamide;
(4-(4-(((R)-1-(3-(difluoromethyl)-2-fluorophenyl)ethyl)amino)quinolin-6-yl)piperazin-1-yl)((S)-tetrahydrofuran-3-yl)methanone;
(4-(4-(((R)-1-(3-(difluoromethyl)-2-fluorophenyl)ethyl)amino)quinolin-6-yl)piperazin-1-yl)((R)-tetrahydrofuran-3-yl)methanone;
(R)-(4-(4-((1-(3-(difluoromethyl)-2-fluorophenyl)ethyl)amino)quinolin-6-yl)piperazin-1-yl)(oxetan-3-yl)methanone;
(R)-1-(4-(4-((1-(3-(difluoromethyl)-2-fluorophenyl)ethyl)amino)quinolin-6-yl)piperazin-1-yl)-2-methylpropan-1-one;
6-((S)-3-aminopyrrolidin-1-yl)-N—((R)-1-(3-(difluoromethyl)-2-fluorophenyl)ethyl)quinolin-4-amine;
6-(6-oxa-3-azabicyclo[3.1.1]heptan-3-yl)-N—((R)-1-(2-methyl-3-(trifluoromethyl)phenyl)ethyl)quinolin-4-amine;
6-((R)-3-aminopyrrolidin-1-yl)-N—((R)-1-(2-methyl-3-(trifluoromethyl)phenyl)ethyl)quinolin-4-amine;
(R)-6-methyl-2-(4-((1-(2-methyl-3-(trifluoromethyl)phenyl)ethyl)amino)quinolin-6-yl)-2,6-diazaspiro[3.4]octan-5-one;
6-((1 S,4S)-2-oxa-5-azabicyclo[2.2.1]heptan-5-yl)-N—((R)-1-(2-methyl-3-(trifluoromethyl)phenyl)ethyl)quinolin-4-amine;
(R)-2-methyl-8-(4-((1-(2-methyl-3-(trifluoromethyl)phenyl)ethyl)amino)quinolin-6-yl)-2,8-diazaspiro[4.5]decan-1-one;
(R)-6-(4-(1-methyl-1H-pyrazol-4-yl)piperazin-1-yl)-N-(1-(2-methyl-3-(trifluoromethyl)phenyl)ethyl)quinolin-4-amine;
(R)-6-(5,6-dihydro-[1,2,4]triazolo[1,5-a]pyrazin-7(8H)-yl)-N-(1-(2-methyl-3-(trifluoromethyl)phenyl)ethyl)quinolin-4-amine;
(R)-cyclopropyl(4-(4-((1-(2-methyl-3-(trifluoromethyl)phenyl)ethyl)amino)quinolin-6-yl)piperazin-1-yl)methanone;
(R)-1-(4-(4-((1-(2-methyl-3-(trifluoromethyl)phenyl)ethyl)amino)quinolin-6-yl)piperazin-1-yl)ethan-1-one;
(R)—N-(1-(2-methyl-3-(trifluoromethyl)phenyl)ethyl)-6-(4-(methylamino)piperidin-1-yl)quinolin-4-amine;
(R)-(4-(4-((1-(2-methyl-3-(trifluoromethyl)phenyl)ethyl)amino)quinolin-6-yl)piperazin-1-yl)(oxetan-3-yl)methanone;
6-((R)-3-aminopyrrolidin-1-yl)-N—((R)-1-(3-(difluoromethyl)-2-fluorophenyl)ethyl)-8-fluoroquinolin-4-amine;
N—((R)-1-(3-(difluoromethyl)-2-fluorophenyl)ethyl)-6-((R)-3-(dimethylamino)pyrrolidin-1-yl)quinolin-4-amine;
(R)-6-(4-(dimethylamino)piperidin-1-yl)-N-(1-(2-methyl-3-(trifluoromethyl)phenyl)ethyl)quinolin-4-amine;
1-((S)-2-methyl-4-(4-(((R)-1-(2-methyl-3-(trifluoromethyl)phenyl)ethyl)amino)quinolin-6-yl)piperazin-1-yl)ethan-1-one;
(R)—N-(1-(2-methyl-3-(trifluoromethyl)phenyl)ethyl)-6-(4-(thiazol-2-yl)piperazin-1-yl)quinolin-4-amine;
(R)-6-(3,3-dimethylpiperazin-1-yl)-N-(1-(2-methyl-3-(trifluoromethyl)phenyl)ethyl)quinolin-4-amine;
(R)-1-(2,2-dimethyl-4-(4-((1-(2-methyl-3-(trifluoromethyl)phenyl)ethyl)amino)quinolin-6-yl)piperazin-1-yl)ethan-1-one;
(R)—N-(1-(2-methyl-3-(trifluoromethyl)phenyl)ethyl)-6-morpholinoquinolin-4-amine;
N—((R)-1-(4-(((R)-1-(2-methyl-3-(trifluoromethyl)phenyl)ethyl)amino)quinolin-6-yl)pyrrolidin-3-yl)acetamide;
N—((S)-1-(4-(((R)-1-(2-methyl-3-(trifluoromethyl)phenyl)ethyl)amino)quinolin-6-yl)pyrrolidin-3-yl)acetamide;
(R)-1-(4-(4-((1-(3-(difluoromethyl)-2-fluorophenyl)ethyl)amino)quinolin-6-yl)-4-hydroxypiperidin-1-yl)ethan-1-one;
(R)-1-(4-(4-((1-(3-(difluoromethyl)-2-fluorophenyl)ethyl)amino)quinolin-6-yl)-4-fluoropiperidin-1-yl)ethan-1-one;
(R)-1-(4-(((R)-1-(3-(difluoromethyl)-2-fluorophenyl)ethyl)amino)quinolin-6-yl)pyrrolidin-3-ol;
(S)-1-(4-(((R)-1-(3-(difluoromethyl)-2-fluorophenyl)ethyl)amino)quinolin-6-yl)pyrrolidin-3-ol;
N—((R)-1-(3-(difluoromethyl)-2-fluorophenyl)ethyl)-6-((R)-3-(methylamino)pyrrolidin-1-yl)quinolin-4-amine;
(R)—N-(1-(2-methyl-3-(trifluoromethyl)phenyl)ethyl)-6-(3-methyl-5,6-dihydro-[1,2,4]triazolo[4,3-a]pyrazin-7(8H)-yl)quinolin-4-amine;
(R)-2-methyl-1-(4-(4-((1-(2-methyl-3-(trifluoromethyl)phenyl)ethyl)amino)quinolin-6-yl)piperazin-1-yl)propan-1-one;
(R)-(4-(7-methoxy-4-((1-(2-methyl-3-(trifluoromethyl)phenyl)ethyl)amino)quinolin-6-yl)piperazin-1-yl)(oxetan-3-yl)methanone;
(R)—N-(1-(3-(difluoromethyl)-2-fluorophenyl)ethyl)-6-morpholinoquinolin-4-amine;
(R)-6-(4-aminopiperidin-1-yl)-N-(1-(3-(difluoromethyl)-2-fluorophenyl)ethyl)quinolin-4-amine;
N—((R)-1-(2-methyl-3-(trifluoromethyl)phenyl)ethyl)-6-((R)-3-methylpiperazin-1-yl)quinolin-4-amine;
1-((R)-2-methyl-4-(4-(((R)-1-(2-methyl-3-(trifluoromethyl)phenyl)ethyl)amino)quinolin-6-yl)piperazin-1-yl)ethan-1-one;
(R)—N-(1-(2-methyl-3-(trifluoromethyl)phenyl)ethyl)-6-(4-(2,2,2-trifluoroethyl)piperazin-1-yl)quinolin-4-amine;
(4-(4-(((R)-1-(2-methyl-3-(trifluoromethyl)phenyl)ethyl)amino)quinolin-6-yl)piperazin-1-yl)(tetrahydrofuran-2-yl)methanone;
6-((2R,5S)-2,5-dimethylpiperazin-1-yl)-N—((R)-1-(2-methyl-3-(trifluoromethyl)phenyl)ethyl)quinolin-4-amine;
(R)-7-methoxy-N-(1-(2-methyl-3-(trifluoromethyl)phenyl)ethyl)-6-(piperazin-1-yl)quinolin-4-amine;
6-((2S,5R)-2,5-dimethylpiperazin-1-yl)-N—((R)-1-(2-methyl-3-(trifluoromethyl)phenyl)ethyl)quinolin-4-amine;
1-((2S,5R)-2,5-dimethyl-4-(4-(((R)-1-(2-methyl-3-(trifluoromethyl)phenyl)ethyl)amino)quinolin-6-yl)piperazin-1-yl)ethan-1-one;
6-(3,6-diazabicyclo[3.1.1]heptan-3-yl)-N—((R)-1-(2-methyl-3-(trifluoromethyl)phenyl)ethyl)quinolin-4-amine;
6-(3,8-diazabicyclo[3.2.1]octan-3-yl)-N—((R)-1-(2-methyl-3-(trifluoromethyl)phenyl)ethyl)quinolin-4-amine;
N—((R)-1-(2-methyl-3-(trifluoromethyl)phenyl)ethyl)-6-((S)-2-methylpiperazin-1-yl)quinolin-4-amine;
1-((S)-3-methyl-4-(4-(((R)-1-(2-methyl-3-(trifluoromethyl)phenyl)ethyl)amino)quinolin-6-yl)piperazin-1-yl)ethan-1-one;
(R)-1-(4-(4-((1-(2-methyl-3-(trifluoromethyl)phenyl)ethyl)amino)quinolin-6-yl)-1,4-diazepan-1-yl)ethan-1-one;
(R)-6-(1,4-diazepan-1-yl)-N-(1-(2-methyl-3 (trifluoromethyl)phenyl)ethyl)quinolin-4-amine;
1-(3-(4-(((R)-1-(2-methyl-3-(trifluoromethyl)phenyl)ethyl)amino)quinolin-6-yl)-3,6-diazabicyclo[3.1.1]heptan-6-yl)ethan-1-one;
(R)-6-(4-(azetidin-1-yl)piperidin-1-yl)-N-(1-(2-methyl-3-(trifluoromethyl)phenyl)ethyl)quinolin-4-amine;
1-((2R,5S)-2,5-dimethyl-4-(4-(((R)-1-(2-methyl-3-(trifluoromethyl)phenyl)ethyl)amino)quinolin-6-yl)piperazin-1-yl)ethan-1-one;
1-((R)-3-methyl-4-(4-(((R)-1-(2-methyl-3-(trifluoromethyl)phenyl)ethyl)amino)quinolin-6-yl)piperazin-1-yl)ethan-1-one;
N—((R)-1-(2-methyl-3-(trifluoromethyl)phenyl)ethyl)-6-((R)-2-methylpiperazin-1-yl)quinolin-4-amine;
(R)—N-(1-(2-methyl-3-(trifluoromethyl)phenyl)ethyl)-6-(4,7-diazaspiro[2.5]octan-7-yl)quinolin-4-amine;
(R)-1-(7-(4-((1-(2-methyl-3-(trifluoromethyl)phenyl)ethyl)amino)quinolin-6-yl)-4,7-diazaspiro[2.5]octan-4-yl)ethan-1-one;
(R)-1-(4-hydroxy-4-(7-methoxy-4-((1-(2-methyl-3-(trifluoromethyl)phenyl)ethyl)amino)quinolin-6-yl)piperidin-1-yl)ethan-1-one;
Methyl (R)-4-fluoro-4-(7-methoxy-4-((1-(2-methyl-3-(trifluoromethyl)phenyl)ethyl)amino)quinolin-6-yl)piperidine-1-carboxylate;
1-((R)-3-(4-(((R)-1-(3-(difluoromethyl)-2-fluorophenyl)ethyl)amino)quinolin-6-yl)-3-methoxypyrrolidin-1-yl)ethan-1-one;
1-((S)-3-(4-(((R)-1-(3-(difluoromethyl)-2-fluorophenyl)ethyl)amino)quinolin-6-yl)-3-methoxypyrrolidin-1-yl)ethan-1-one;
(R)-3-(4-methoxy-4-(4-((1-(2-methyl-3-(trifluoromethyl)phenyl)ethyl)amino)quinolin-6-yl)piperidin-1-yl)-3-oxopropanenitrile;
(R)-2-fluoro-1-(4-methoxy-4-(4-((1-(2-methyl-3-(trifluoromethyl)phenyl)ethyl)amino)quinolin-6-yl)piperidin-1-yl)ethan-1-one;
(R)-1-(4-methoxy-4-(4-((1-(2-methyl-3-(trifluoromethyl)phenyl)ethyl)amino)quinolin-6-yl)piperidin-1-yl)ethan-1-one;
(R)-1-(4-methoxy-4-(4-((1-(2-methyl-3-(trifluoromethyl)phenyl)ethyl)amino)quinolin-6-yl)piperidin-1-yl)-2-methylpropan-1-one;
(R)-1-(4-(4-((1-(3-(difluoromethyl)-2-fluorophenyl)ethyl)amino)quinolin-6-yl)-4-fluoropiperidin-1-yl)-2-methylpropan-1-one;
(R)-1-(4-fluoro-4-(4-((1-(2-methyl-3-(trifluoromethyl)phenyl)ethyl)amino)quinolin-6-yl)piperidin-1-yl)-2-methylpropan-1-one;
1-(3-(4-(((R)-1-(3-(difluoromethyl)-2-fluorophenyl)ethyl)amino)quinolin-6-yl)-3-hydroxypyrrolidin-1-yl)ethan-1-one;
1-(4-(4-((1-(3-(difluoromethyl)-2-fluorophenyl)ethyl)amino)quinolin-6-yl)-4-fluoropiperidin-1-yl)ethan-1-one;
1-((R)-3-(4-(((R)-1-(3-(difluoromethyl)-2-fluorophenyl)ethyl)amino)quinolin-6-yl)-3-fluoropyrrolidin-1-yl)ethan-1-one;
3-((R)-1-((6-((R)-1-acetyl-3-methoxypyrrolidin-3-yl)quinolin-4-yl)amino)ethyl)-2-methylbenzonitrile;
3-((R)-1-((6-((S)-1-acetyl-3-methoxypyrrolidin-3-yl)quinolin-4-yl)amino)ethyl)-2-methylbenzonitrile;
methyl (S)-3-(4-(((R)-1-(3-(difluoromethyl)-2-fluorophenyl)ethyl)amino)quinolin-6-yl)-3-methoxypyrrolidine-1-carboxylate;
methyl (R)-3-(4-(((R)-1-(3-(difluoromethyl)-2-fluorophenyl)ethyl)amino)quinolin-6-yl)-3-methoxypyrrolidine-1-carboxylate;
1-((R)-3-(4-(((R)-1-(3-(difluoromethyl)-2-fluorophenyl)ethyl)amino)quinolin-6-yl)-3-methoxypyrrolidin-1-yl)-2-methylpropan-1-one;
1-((S)-3-(4-(((R)-1-(3-(difluoromethyl)-2-fluorophenyl)ethyl)amino)quinolin-6-yl)-3-methoxypyrrolidin-1-yl)-2-methylpropan-1-one;
1-((S)-3-(4-(((R)-1-(3-(difluoromethyl)-2-fluorophenyl)ethyl)amino)quinolin-6-yl)-3-(methoxy-d3)pyrrolidin-1-yl)ethan-1-one;
1-((R)-3-(4-(((R)-1-(3-(difluoromethyl)-2-fluorophenyl)ethyl)amino)quinolin-6-yl)-3-(methoxy-d3)pyrrolidin-1-yl)ethan-1-one;
cyclopropyl((R)-3-(4-(((R)-1-(3-(difluoromethyl)-2-fluorophenyl)ethyl)amino)quinolin-6-yl)-3-methoxypyrrolidin-1-yl)methanone;
cyclopropyl((S)-3-(4-(((R)-1-(3-(difluoromethyl)-2-fluorophenyl)ethyl)amino)quinolin-6-yl)-3-methoxypyrrolidin-1-yl)methanone;
(R)-1-(3-(4-((1-(3-(difluoromethyl)-2-fluorophenyl)ethyl)amino)quinolin-6-yl)-2,5-dihydro-1H-pyrrol-1-yl)ethan-1-one;
(S)-3-(4-(((R)-1-(3-(difluoromethyl)-2-fluorophenyl)ethyl)amino)quinolin-6-yl)-3-methoxy-N,N-dimethylpyrrolidine-1-carboxamide;
(R)-3-(4-(((R)-1-(3-(difluoromethyl)-2-fluorophenyl)ethyl)amino)quinolin-6-yl)-3-methoxy-N,N-dimethylpyrrolidine-1-carboxamide;
methyl (R)-4-(4-((1-(3-(difluoromethyl)-2-fluorophenyl)ethyl)amino)quinolin-6-yl)-4-methoxypiperidine-1-carboxylate;
(R)-3-(4-(((R)-1-(3-(difluoromethyl)-2-fluorophenyl)ethyl)amino)quinolin-6-yl)-3-methoxy-N-methylpyrrolidine-1-carboxamide;
(S)-3-(4-(((R)-1-(3-(difluoromethyl)-2-fluorophenyl)ethyl)amino)quinolin-6-yl)-3-methoxy-N-methylpyrrolidine-1-carboxamide;
((S)-3-(4-(((R)-1-(3-(difluoromethyl)-2-fluorophenyl)ethyl)amino)quinolin-6-yl)-3-methoxypyrrolidin-1-yl)(morpholino)methanone;
((R)-3-(4-(((R)-1-(3-(difluoromethyl)-2-fluorophenyl)ethyl)amino)quinolin-6-yl)-3-methoxypyrrolidin-1-yl)(morpholino)methanone;
azetidin-1-yl((R)-3-(4-(((R)-1-(3-(difluoromethyl)-2-fluorophenyl)ethyl)amino)quinolin-6-yl)-3-methoxypyrrolidin-1-yl)methanone;
azetidin-1-yl((S)-3-(4-(((R)-1-(3-(difluoromethyl)-2-fluorophenyl)ethyl)amino)quinolin-6-yl)-3-methoxypyrrolidin-1-yl)methanone;
((S)-3-(4-(((R)-1-(3-(difluoromethyl)-2-fluorophenyl)ethyl)amino)quinolin-6-yl)-3-methoxypyrrolidin-1-yl)(pyrrolidin-1-yl)methanone;
((R)-3-(4-(((R)-1-(3-(difluoromethyl)-2-fluorophenyl)ethyl)amino)quinolin-6-yl)-3-methoxypyrrolidin-1-yl)(pyrrolidin-1-yl)methanone;
(R)-3-(4-(((R)-1-(3-(difluoromethyl)-2-fluorophenyl)ethyl)amino)quinolin-6-yl)-N-isopropyl-3-methoxypyrrolidine-1-carboxamide;
(S)-3-(4-(((R)-1-(3-(difluoromethyl)-2-fluorophenyl)ethyl)amino)quinolin-6-yl)-N-isopropyl-3-methoxypyrrolidine-1-carboxamide;
(S)-3-(4-(((R)-1-(3-(difluoromethyl)-2-fluorophenyl)ethyl)amino)quinolin-6-yl)-N-isobutyl-3-methoxypyrrolidine-1-carboxamide;
(R)-3-(4-(((R)-1-(3-(difluoromethyl)-2-fluorophenyl)ethyl)amino)quinolin-6-yl)-N-isobutyl-3-methoxypyrrolidine-1-carboxamide;
(R)—N-cyclopropyl-3-(4-(((R)-1-(3-(difluoromethyl)-2-fluorophenyl)ethyl)amino)quinolin-6-yl)-3-methoxypyrrolidine-1-carboxamide;
(S)—N-cyclopropyl-3-(4-(((R)-1-(3-(difluoromethyl)-2-fluorophenyl)ethyl)amino)quinolin-6-yl)-3-methoxypyrrolidine-1-carboxamide;
(S)—N-cyclobutyl-3-(4-(((R)-1-(3-(difluoromethyl)-2-fluorophenyl)ethyl)amino)quinolin-6-yl)-3-methoxypyrrolidine-1-carboxamide;
(R)—N-cyclobutyl-3-(4-(((R)-1-(3-(difluoromethyl)-2-fluorophenyl)ethyl)amino)quinolin-6-yl)-3-methoxypyrrolidine-1-carboxamide;
isopropyl (S)-3-(4-(((R)-1-(3-(difluoromethyl)-2-fluorophenyl)ethyl)amino)quinolin-6-yl)-3-methoxypyrrolidine-1-carboxylate;
isopropyl (R)-3-(4-(((R)-1-(3-(difluoromethyl)-2-fluorophenyl)ethyl)amino)quinolin-6-yl)-3-methoxypyrrolidine-1-carboxylate;
cyclobutyl (R)-3-(4-(((R)-1-(3-(difluoromethyl)-2-fluorophenyl)ethyl)amino)quinolin-6-yl)-3-methoxypyrrolidine-1-carboxylate;
cyclobutyl (S)-3-(4-(((R)-1-(3-(difluoromethyl)-2-fluorophenyl)ethyl)amino)quinolin-6-yl)-3-methoxypyrrolidine-1-carboxylate. 1-((S)-3-(4-(((R)-1-(3-(difluoromethyl)-2-fluorophenyl)ethyl)amino)quinolin-6-yl-2-d)-3-methoxypyrrolidin-1-yl)ethan-1-one;
1-((R)-3-(4-(((R)-1-(3-(difluoromethyl)-2-fluorophenyl)ethyl)amino)quinolin-6-yl-2-d)-3-methoxypyrrolidin-1-yl)ethan-1-one;
1-((R)-3-(4-(((R)-1-(3-(difluoromethyl)-2-fluorophenyl)ethyl)amino)quinolin-6-yl-2-d)-3-methoxypyrrolidin-1-yl)-2-methylpropan-1-one;
1-((S)-3-(4-(((R)-1-(3-(difluoromethyl)-2-fluorophenyl)ethyl)amino)quinolin-6-yl-2-d)-3-methoxypyrrolidin-1-yl)-2-methylpropan-1-one;
cyclopropyl((S)-3-(4-(((R)-1-(3-(difluoromethyl)-2-fluorophenyl)ethyl)amino)quinolin-6-yl-2-d)-3-methoxypyrrolidin-1-yl)methanone;
cyclopropyl((R)-3-(4-(((R)-1-(3-(difluoromethyl)-2-fluorophenyl)ethyl)amino)quinolin-6-yl-2-d)-3-methoxypyrrolidin-1-yl)methanone;
(R)-3-(4-(((R)-1-(3-(difluoromethyl)-2-fluorophenyl)ethyl)amino)quinolin-6-yl-2-d)-3-methoxy-N-methylpyrrolidine-1-carboxamide;
(S)-3-(4-(((R)-1-(3-(difluoromethyl)-2-fluorophenyl)ethyl)amino)quinolin-6-yl-2-d)-3-methoxy-N-methylpyrrolidine-1-carboxamide;
(S)-3-(4-(((R)-1-(3-(difluoromethyl)-2-fluorophenyl)ethyl)amino)quinolin-6-yl-2-d)-3-methoxy-N,N-dimethylpyrrolidine-1-carboxamide; and
(R)-3-(4-(((R)-1-(3-(difluoromethyl)-2-fluorophenyl)ethyl)amino)quinolin-6-yl-2-d)-3-methoxy-N,N-dimethylpyrrolidine-1-carboxamide.

48. A pharmaceutical composition comprising the compound according to claim 1 and at least one additional component chosen from pharmaceutically acceptable carriers, pharmaceutically acceptable vehicles, and pharmaceutically acceptable excipients.

49. The pharmaceutical composition according to claim 48, wherein the compound is present in a therapeutically effective amount.

50. A method for inhibiting SOS1 activity in a cell, comprising contacting the cell in which inhibition of SOS1 is desired with a therapeutically effective amount of a compound according to claim 1.

51. The method of claim 50, wherein the cell has an activating mutation in a RAS family-member gene.

52. The method of claim 51, wherein the cell has an activating mutation in an SOS1 gene.

53. A method of treating cancer in a subject in need thereof, comprising administering to the subject an effective amount of the compound according to claim 1, wherein the cancer is chosen from breast cancer, lung cancer, pancreatic cancer, small bowel cancer, colorectal cancer, gall bladder cancer, thyroid cancer, liver cancer, bile duct cancer, ovarian cancer, endometrial cancer, cervical cancer, bladder cancer, prostate cancer, esophageal cancer, and blood cancer.

54. The method according to claim 53, wherein the cancer is lung cancer.

55. The method according to claim 53, wherein the cancer is non-small cell lung cancer.

56. The method according to claim 53, wherein the cancer is pancreatic cancer.

57. The method according to claim 53, wherein the cancer is colorectal cancer.
